SEQUENCE LISTING (19 sequences)

SEQ ID NO: 1 - protein, 488 aa, Artificial Sequence
  Synthetic peptide
MRKRAPQSEMAPAGVSLRATILCCLLAWAGLAAGDRVYIHIPFHLVIHNESTCEQLAKANAGKPKDPTFIPAPIQAKTSPVDEKALQDQLVLVAAKLDTEDKLRAAMVGMLANFLGFRIYGMHSELWGVVHGATVLSPTAVFGTLASLYLGALDHTADRLQAILGVPWKDKNCTSRLDAHKVLSALQAVAQGLLVAQGRADSQAQLLLSTVVGVFTAPGLHLKQPFVQGLALYTPVVLPRSLDFTELDVAAEKIDRFMQAVTGWKTGCSLMGASVDSTLAFNTYVHFQGKMKGFSLLAEPQEFWVDNSTSVSVPMLSGMGTFQHWSDIQDNFSVTQVPFTESACLLLIQPHYASDLDKVEGLTFQQNSLNWMKKLSPRTIHLTMPQLVLQGSYDLQDLLAQAELPAILHTELNLQKLSNDRIRVGEVLNSIFFELEADEREPTESTQQLNKPEVLEVTLNRPFLFAVYDQSATALHFLGRVANPLSTA

SEQ ID NO: 2 - protein, 11 aa, Artificial Sequence
  Synthetic peptide
VEIPGWFNDQK

SEQ ID NO: 3 - protein, 16 aa, Artificial Sequence
  Synthetic peptide
GVVGHPVTIPCTYSTR

SEQ ID NO: 4 - protein, 9 aa, Artificial Sequence
  Synthetic peptide
PLQETECTF

SEQ ID NO: 5 - protein, 16 aa, Artificial Sequence
  Synthetic peptide
VDCGYPTVTSEQCNNR

SEQ ID NO: 6 - protein, 16 aa, Artificial Sequence
  Synthetic peptide
GCCFDSSIPNVPWCFK

SEQ ID NO: 7 - protein, 10 aa, Artificial Sequence
  Synthetic peptide
LVQEVTDFAK

SEQ ID NO: 8 - protein, 13 aa, Artificial Sequence
  Synthetic peptide
LGEYGFQNAVLVR

SEQ ID NO: 9 - protein, 11 aa, Artificial Sequence
  Synthetic peptide
FPNAEFAEITK

SEQ ID NO: 10 - protein, 13 aa, Artificial Sequence
  Synthetic peptide
GLVLIAFSQYLQK

SEQ ID NO: 11 - protein, 13 aa, Artificial Sequence
  Synthetic peptide
DVFLGTFLYEYSR

SEQ ID NO: 12 - protein, 9 aa, Artificial Sequence
  Synthetic peptide
TPQIQVYSR

SEQ ID NO: 13 - protein, 11 aa, Artificial Sequence
  Synthetic peptide
ALDFAVSEYNK

SEQ ID NO: 14 - protein, 17 aa, Artificial Sequence
  Synthetic peptide
LLGAPQEADASEEGVQR

SEQ ID NO: 15 - protein, 9 aa, Artificial Sequence
  Synthetic peptide
GSNDAYHSR

SEQ ID NO: 16 - protein, 10 aa, Artificial Sequence
  Synthetic peptide
SLLNSLEEAK

SEQ ID NO: 17 - protein, 12 aa, Artificial Sequence
  Synthetic peptide
ASGIIDTLFQDR

SEQ ID NO: 18 - protein, 17 aa, Artificial Sequence
  Synthetic peptide
LFDSDPITVVLPEEVSK

SEQ ID NO: 19 - protein, 12 aa, Artificial Sequence
  Synthetic peptide
ALQDQLVLVAAK

(19) United States
(12) Patent Application Publication
(10) Pub. No.: US 2014/0038203 A1
(43) Pub. Date: Feb. 6, 2014

(54) METHODS FOR DETECTING OR PREDICTING KIDNEY DISEASE

(71) Applicant: MUSC FOUNDATION FOR RESEARCH DEVELOPMENT, Charleston, SC (US)

(72) Inventors: John ARTHUR, Mount Pleasant, SC (US); Michael JANECH, Charleston, SC (US); Joseph ALGE, Charleston, SC (US)

(73) Assignee: MUSC FOUNDATION FOR RESEARCH DEVELOPMENT, Charleston, SC (US)

(21) Appl. No.: 13/937,967

(22) Filed: Jul. 9, 2013

Related U.S. Application Data
(60) Provisional application No. 61/669,519, filed on Jul. 9, 2012.

(51) Int. Cl. G01N 33/68

(52) U.S. Cl. CPC G01N 33/6893
USPC 435/7.4; 435/7.1; 435/7.92; 435/23; 530/389.2

(57) ABSTRACT

Methods of detecting or predicting the onset or magnitude of kidney diseases such as acute kidney disease (AKI), previously called acute renal failure (ARF), are provided. In various aspects, methods and kits are provided to detect specific urinary proteins associated with AKI diagnosis or prognosis such as, e.g., angiotensinogen.

This application claims the benefit of U.S. Provisional Patent Application No. 61/669,519, filed Jul. 9, 2012, the entirety of which is incorporated herein by reference.

This invention was made with government support under R01DK080234 and UL1 RR029882 awarded by the National Institutes of Health and a Merit Review award from the Biomedical Laboratory Research and Development Program of the Department of Veterans Affairs. The government has certain rights in the invention.

The sequence listing that is contained in the file named “MESCP067US_ST25.txt”, which is 9 KB (as measured in Microsoft Windows®) and was created on Jul. 9, 2013, is filed herewith by electronic submission and is incorporated by reference herein.

### BACKGROUND OF THE INVENTION

1. Field of the Invention

The present invention relates generally to the fields of molecular biology and medicine. More particularly, it concerns methods for predicting the severity or onset of acute kidney injury.

2. Description of Related Art

Acute kidney injury (AKI) is a common and serious medical condition that is associated with adverse outcomes. Epidemiologic studies have reported that it is observed in 2 to 10% of hospitalized patients, and its incidence is increasing (Waikar et al., 2006; Nash et al., 2002; Lafrance and Miller, 2010). The high prevalence of AKI is a reflection of its multifactorial nature. The most common contributing causes are sepsis, major surgery (especially cardiac surgery), renal ischemic injury (including hypovolemia, hypotension, cardiac disease) and nephrotoxic agents (Nash et al., 2002; Uchino et al., 2005; Mehta et al., 2004) although many other types of insult can produce AKI. Despite advances in understanding of the disease and in patient care, reported in-hospital mortality rate attributed to AKI remain high, ranging from 20% to 60% (Waikar et al., 2006; Uchino et al., 2005; Mehta et al., 2004). Furthermore, severe AKI requiring renal replacement therapy has been identified as an independent risk factor for mortality, and it is now recognized that even mild AKI increases long-term risk of death, even long after discharge (Lafrance and Miller, 2010; Chertow et al., 1998; Loef et al., 2005). Additionally, patients who survive AKI have longer hospital stays, incur significantly more healthcare costs, and are at increased risk of developing chronic kidney disease and end-stage renal disease (Chertow et al., 2005; Venkatachalam et al., 2010; Coca et al., 2009; Lo et al., 2009).

One of the most important factors underlying the poor outcomes seen in AKI patients is the current method of diagnosis, which is based upon either an increase in serum creatinine (sCr) or decreased urine output (UO) (Bellomo et al., 2004; Mehta et al., 2007). However, sCr reflects glomerular filtration, not renal injury, and consequently the use of creatinine as a surrogate marker of AKI results in diagnosis after an appreciable loss in renal function has already occurred (Cruz et al., 2009; Ricci et al., 2011). Furthermore, sCr and UO values at the time of diagnosis are of limited prognostic value, making it difficult to discriminate between mild and severe AKI and to predict important outcomes such as the need for renal replacement therapy (RRT) and mortality. For these reasons, the need for better biomarkers of AKI has been recognized as a crucial barrier to improvement of the outcomes of AKI patients. Several biomarkers have been proposed in the literature. The most well-studied are kidney injury molecule 1 (KIM-1), neutrophil gelatinase associated lipocalin (NGAL), interleukin-18 (IL-18), cystatin C (Cys-C), and liver fatty acid binding protein (L-FABP) (Han et al., 2002; Mishra et al., 2003; Mishra et al., 2005; Melnikov et al., 2001; Parikh et al., 2004; Herget-Rosenthal et al., 2004; Portilla et al., 2008). Notably, these biomarkers initially appeared capable of early, accurate detection of AKI, but subsequent verification studies have reported lower accuracy (Liangos et al., 2009; Koyner et al., 2010; Wagener et al., 2008; Parikh et al., 2006; Haase et al., 2008; Koyner et al., 2008; Parikh et al., 2011a; Parikh et al., 2011b). Additionally, the emphasis on early detection has been to the exclusion of the investigation of their prognostic predictive power, and the limited data available on the prognostic value of these biomarkers suggests that they are better suited to early diagnosis than prediction of adverse outcomes (Hall et al., 2011; Koyner et al., 2012). The limitations of previously identified individual biomarkers underscore the need to discover novel biomarkers, particularly with regard to prognosis. Novel biomarkers could be used in combination with existing ones to augment the sensitivity and specificity of clinical tests used to predict AKI diagnosis and outcomes. Furthermore, they could improve understanding of the molecular pathobiology of AKI and possibly lead to the development of novel therapeutic approaches. Clearly, there is a need for new methods to identify AKI.

### SUMMARY OF THE INVENTION

The present invention overcomes limitations in the prior art by providing new methods for predicting the onset, progression, or severity of kidney disease such as acute kidney injury (AKI). In some aspects, one or more proteins from a biological sample such as a urine sample may be used to predict the onset, progression, or severity of AKI.

An aspect of the present invention relates to a method for determining an increased risk of developing a nephropathy or kidney disease in a subject, comprising measuring at least one biomarker protein in a urine sample from said subject, wherein said biomarker protein is selected from the group consisting of (a) angiotensinogen, apolipoprotein A-IV, pigment epithelium-derived factor, thymosin β4, insulin-like growth factor-binding protein 1, myoglobin, vitamin D binding protein, complement C4-B, profilin-1, alpha-1 antitrypsin, fibrinogen alpha chain, glutathione peroxidase 3, superoxide dismutase [Cu—Zn], complement C3, antithrombin III, neutrophil defensin 1; and (b) non-secretory ribonuclease, secreted Ly-6/uPAR-related protein 1, pro-epidermal growth factor precursor (pro-EGF protein), and CD59 glycoprotein; wherein an increase in level of a protein from group (a) or a decrease in level of a protein from group (b) in said urine sample relative to a reference level indicates that the subject has an increased risk of developing the nephropathy or kidney disease. In some embodiments, said protein is selected from the group consisting of: (a) apolipoprotein A-IV, thymosin β4, insulin-like growth factor-binding protein 1, vitamin D binding protein, profilin-1, glutathione peroxidase 3, superoxide dismutase [Cu—Zn], neutrophil defensin 1, and (b) non-secretory ribonuclease, secreted Ly-6/uPAR-related protein 1, pro-epidermal growth factor precursor (pro-EGF protein), and CD59 glycoprotein. In some embodiments, the method further comprises administering a kidney therapy or kidney therapeutic to the subject if the subject has an increased risk of developing the nephropathy or kidney disease. The kidney therapy may be, e.g., early dialysis, a peptide therapeutic (e.g., alpha-MSH), fenoldopam, dopamine, erythropoietin (EPO), a small molecule therapeutic, a protein therapeutic, hemofiltration, hemodialysis, or continuous renal replacement therapy (CRRT). In some embodiments, said measuring occurs within less than or equal to 24 hours after the subject has sustained an injury, such as a kidney injury. In some embodiments, said measuring occurs within 24-48 hours, or after 24 hours, after the subject has sustained an injury, such as a kidney injury.

The method may further comprise preparing a report of said measuring. The nephropathy or kidney disease may be acute kidney injury (AKI), a progressive or worsening acute kidney injury, or a diabetic nephropathy, acute tubular necrosis, acute interstitial nephritis, a glomerulonephropathy, a glomerulonephritis, a renal vasculitis, an obstruction of the renal artery, a renal ischemic injury, a tumor lysis syndrome, rhabdomyolysis, a urinary tract obstruction, a prerenal azotemia, a renal vein thrombosis, a cardiorenal syndrome, a hepatorenal syndrome, a pulmonary-renal syndrome, an abdominal compartment syndrome, an injury from a nephrotoxic agent, or a contrast nephropathy. The nephropathy or kidney disease may be a pre-AKI disease. The subject may be at an increased risk for an AKI. an The subject may have an AKI that has not been diagnosed. The protein may be angiotensinogen. The reference level may be an angiotensinogen concentration such as, e.g., at least about 12 ng/ml, at least about 25 ng/ml, at least about 50 ng/ml. The method may further comprise measuring creatinine concentration in the urine sample. Said measuring may comprise measuring the urine angiotensinogen to creatinine ratio (uAnCR), wherein an increase in the uAnCR relative to a reference level indicates that the subject has an increased risk of severe AKI. The reference level may be a uANCR such as, e.g., at least about 15 ng/mg, at least about 26 ng/mg, at least about 50 ng/mg. Alternatively, the angiotensinogen level or uAnCR may be at least about 3-fold or at least about 5-fold or at least about 10-fold higher than the level of a reference sample from a subject that does not experience kidney injury or a subject that does not experience severe kidney injury. In some embodiments, a cardiac surgery is or has been performed on the subject. In some embodiments, said measuring comprises measuring 2, 3, 4, 5, 6, 7, 8, 9, 10, 11, 12, 13, 14, 15, 16, 17, or all of the proteins from group (a) and/or group (b). The method may further comprise measuring a second protein in said urine sample, wherein said protein is selected from the group consisting of: (a) lysozyme c and albumin; and (b) uromodulin, hepcidin and polymeric immunoglobulin receptor; wherein an increase in level of a protein from group (a) or a decrease in level of a protein from group (b) in said urine sample relative to a reference level indicates that the subject has an increased risk of developing acute kidney injury. The method may also consist of measuring one or more biomarker proteins in group (a) or (b) together with one or more biomarker proteins in the group consisting of (c) neutrophil gelatinase associated lipocalin (NGAL), kidney injury molecule 1 (KIM-1), trefoil factor 3, beta-2-microglobulin, cystatin c, clusterin, calbindin d28, epidermal growth factor, glutathione S-transferase a, glutathione S-transferase μ, osteoactivin, osteopontin, podocin, renal papillary antigen 1, TIMP-1, VEGF, L type-fatty acid binding protein, netrin, fetuin A, alpha-1 microglobulin, beta 2 glycoprotein, plasma retinol binding protein, N-acetyl glucosaminidase (NAG), NHE3, IL-18, IL-6, hepatocyte growth factor, Cyr61, leukemia inhibitory factor, ICAM-1, HSP70, zinc alpha-2 glycoprotein, MCP-1 or another biomarker of kidney injury or nephropathy. The method may comprise measuring a protein in group (a) or (c) and a protein in group (b) to create a ratio of (a) to (b) or (c) to (b). The ratio of (a) to (b) or (c) to (b) may be compared to a reference value to determine the risk of developing new or worsening kidney disease or nephropathy. The biomarker protein ratio may also predict an increased risk of developing acute kidney injury or a worsening acute kidney injury. In some embodiments, the method may further comprise measuring in the blood or urine of the subject 1, 2, 3, 4, 5, 6, 7, 8, 9, or all of NGAL, IL-18, L-FABP, KIM-1, albumin, total protein, beta-2 microglobulin, cystatin c, clusterin, and/or trefoil factor 3.

The method may further comprise measuring urea nitrogen or creatinine in the blood of the subject. The subject may be a human patient. The patient may have diabetes or prediabetes. The risk may comprise worsening of AKI, AKIN stage 2 AKI, AKIN stage 3 AKI, need for renal replacement therapy, or death. The method may further comprise obtaining the urine sample from the subject. The measuring may comprise measuring the protein once or repeatedly in the subject. In some embodiments, an increase in a protein from group (a) or a decrease in level of a protein from group (b) or an increase in the biomarker protein ratio of (a) to (b) or (c) to (b) in a more recently obtained urine sample from the subject relative to a previous level of the protein in the subject indicates that the subject has an increased risk of developing the acute kidney injury or nephropathy. The methods may further comprise measuring one or more additional proteins in the urine sample. In some embodiments, the subject has an acute kidney injury such as, e.g., severe AKI, early AKI, moderate AKI, or a mild AKI. In some embodiments, the subject has substantially no or does not have an acute kidney injury. The subject may be in a clinical trial. The subject may have a diabetic nephropathy, prediabetes, diabetes, acute tubular necrosis, acute interstitial nephritis, a glomerulonephropathy, a glomerulonephritis, a renal vasculitis, an obstruction of the renal artery, sepsis, an infection, a systemic inflammatory response syndrome, a renal ischemic injury, a tumor lysis syndrome, rhabdomyolysis, a urinary tract obstruction, a prerenal azotemia, a renal vein thrombosis, hypovolemia, hypotension, a cardiorenal syndrome, a hepatorenal syndrome, a pulmonary renal syndrome, an abdominal compartment syndrome, a cardiac surgery, a noncardiac surgery, an abdominal cavity surgery, an aneurysm repair surgery, an injury from a nephrotoxic agent, or a contrast nephropathy.

The method may further comprise a method of predicting the occurrence or severity of acute kidney injury in the subject. In some embodiments, the subject has substantially no acute kidney injury when the urine sample is obtained from the subject. In some embodiments, an increased angiotensinogen level in said urine sample relative to a control sample indicates that the subject has an increased risk of requiring dialysis. In some embodiments, an increased biomarker protein level in group (a) or a decrease of a protein in group (b) or an increase in the biomarker protein ratio in said urine relative to a control sample indicates that the subject has an increased risk of death, longer hospitalization or intensive care unit stay duration, and/or of developing chronic kidney disease or more rapid progression of chronic kidney disease. The subject may have a nephropathy or kidney disease. The nephropathy or kidney disease may be worsening renal function or end-stage renal disease. In some embodiments, the patient is administered a therapeutic, and wherein the concentration of said at least one biomarker protein or biomarker protein ratio in the urine relative to one or more previous urinary concentration of said at least one biomarker protein or biomarker protein ratio in the patient is used to determine if therapeutic has altered renal function. The reference level may be determined from a control sample. The method may further comprise monitoring the response to a treatment for acute kidney injury in the patient. The method may further comprise determining if the treatment should be changed. The measuring may comprise mass spectrometry, LC-MS/MS, selective reaction monitoring (SRM), or multiple reaction monitoring (MRM), MALDI-MS/MS, MALDI-MS, surface enhanced laser desorption/ionization (SELDI), or capillary electrophoresis mass spectrometry (CE-MS), or an immunoassay method such as, e.g., an immunohistochemistry assay, a radioimmunoassay (RIA), an immunoradiometric assay, a Western blot analysis, a fluoroimmunoas say, an automated quantitative analysis (AQUA) system assay, spectroscopy, spectrophotometry, a lateral flow assay, a chemiluminescent labeled sandwich assay, a nephelometry assay, and an enzyme-linked immunosorbent assay (ELISA), a chemiluminescent assay, a bioluminescent assay, a gel electrophoresis, or a nephelometry assay.

In some embodiments, the method may comprise determining the renal toxicity of a drug or compound in a test subject or laboratory animal by measuring the biomarker(s) in the urine. The laboratory animal may be a rat or a mouse or a rabbit or a cat or a dog or a pig or a nonhuman primate. For example, the subject may be a subject is a rat, a mouse, a dog, a cat, a pig, a sheep, a rabbit, a guinea pig or a nonhuman primate including, but not limited to a member of the genus Macaca, a rhesus macaque monkey, a cynomolgus (crab-eating) macaque monkey, a marmoset, a tamarin, a spider monkey, an owl monkey, a vervet monkey, a squirrel monkey, a baboon, a chimpanzee, a gorilla or an orangutan. The drug or compound may be in preclinical development. Generally, during the testing process for development of drugs for the treatment of disease, drugs typically tested in nonhuman subjects. These subjects include but are not limited to rats, mice, cats, dogs, pigs, sheep and nonhuman primates. One or more kidney injury biomarkers can be tested in the urine or blood of these nonhuman subjects after the drugs are administered to determine if the drugs cause kidney injury. The drug or compound developer may be a pharmaceutical company or other drug development or testing company. The test may consist of measuring one or more biomarker proteins in the urine. The biomarker protein concentration may be compared to a threshold value or control value. The biomarker may be a single protein or combination of proteins. In some further aspects, the method may further comprise reporting the determination of the biomarker level or interpretation of the level.

In some embodiments, one or more of the protein biomarkers may be directly measured in the kidney tissue of a test subject or laboratory animal as a measurement of renal toxicity or injury. In some embodiments, the method may comprise determining the renal toxicity of a drug or compound in a laboratory animal by measuring the biomarker(s) in kidney tissue. The laboratory animal may be a rat or a mouse or a zebrafish or a rabbit or a cat or a dog or a pig or a nonhuman primate. The drug or compound may be in preclinical development. The drug or compound developer may be a pharmaceutical company or other drug development or testing company. The biomarker measurement may be made by immunohistochemistry. The antibody used for immunohistochemistry may be visualized, e.g., with a fluorescent dye, an enzyme, or colloidal gold. Messenger RNA for the biomarker protein may be measured by in situ hybridization. The biomarker protein may be localized to a specific section of the nephron. The nephron section may be the glomerulus, glomerular podocyte cells, glomerular endothelial cells, glomerular mesangial cells, the proximal convoluted tubule, the brush border of the proximal convoluted tubule cell, the S1 segment of the proximal convoluted tubule, the S2 segment of the proximal convoluted tubule, the pars recta (S3) segment of the proximal tubule, the descending thin loop of Henle, the ascending thin loop of Henle, the medullary portion of the thick ascending loop of Henle, the cortical portion of the thick ascending loop of Henle, the macula densa, the distal convoluted tubule, the connecting segment, the cortical collecting duct, or the medullary collecting duct. In some further aspects, the method may further comprise reporting the determination of the biomarker level or interpretation of the level.

In some embodiments, the method may comprise determining the renal toxicity of a drug or compound in a human. The drug or compound may be administered to the human, e.g., as part of a phase 1, phase 2, a phase 3, or phase 4 clinical trial. The drug or compound developer may be a pharmaceutical company or other drug development or testing company. The test may consist of measuring one or more biomarker proteins in the urine. The biomarker protein concentration may be compared to a threshold value or control value. The biomarker may be a single protein or combination of proteins. In some further aspects, the method may further comprise reporting the determination of the biomarker level or interpretation of the level.

Another aspect of the present invention relates to a method for determining an increased risk of developing a progressing or worsening diabetic nephropathy or kidney disease in a subject, comprising measuring angiotensinogen in a urine sample from said subject, wherein an increased angiotensinogen level in said urine sample relative to a reference level or control sample indicates that the subject has an increased risk of developing the progressing or worsening nephropathy or kidney disease, and wherein the subject has diabetes. The subject may have at least a mild diabetic nephropathy or kidney disease when the urine sample is obtained from the subject. The diabetes may be type 1 diabetes or type 2 diabetes. The method may comprise a method for predicting the progression of a diabetic nephropathy in the subject, wherein an increased angiotensinogen level in said urine sample relative to a control sample indicates that the subject has an increased risk of developing a progressive or worsening nephropathy or kidney disease. In some embodiments, the subject is a human patient. The subject may be a rat, a mouse, a dog, a cat, a pig, a sheep, a rabbit, a guinea pig or a nonhuman primate including, but not limited to a member of the genus Macaca, a rhesus macaque monkey, a cynomolgus (crab-eating) macaque monkey, a marmoset, a tamarin, a spider monkey, an owl monkey, a vervet monkey, a squirrel monkey, a baboon, a chimpanzee, a gorilla or an orangutan. The measuring may be selected from the group consisting of mass spectrometry, multiple reaction monitoring (MRM), selected reaction monitoring, single reaction monitoring, an immunoassay method, an immunohistochemistry assay, a radioimmunoassay (RIA), an immunoradiometric assay, a Western blot analysis, a fluoroimmunoassay, an automated quantitative analysis (AQUA) system assay, spectroscopy, spectrophotometry, a lateral flow assay, a chemiluminescent labeled sandwich assay, an enzyme-linked immunosorbent assay (ELISA), a chemiluminescent assay, a bioluminescent assay, a gel electrophoresis, or a nephelometry assay.

Yet another aspect of the present invention relates to a kit for determining the likelihood of acute kidney injury (AKI) in a mammalian or human subject, comprising an antibody that specifically binds a protein selected from the group consisting of: angiotensinogen, apolipoprotein A-IV, pigment epithelium-derived factor, thymosin β4, insulin-like growth factor-binding protein 1, myoglobin, vitamin D binding protein, complement C4-B, profilin-1, alpha-1 antitrypsin, fibrinogen alpha chain, glutathione peroxidase 3, superoxide dismutase [Cu—Zn], complement C3, antithrombin III, neutrophil defensin 1, and non-secretory ribonuclease, secreted Ly-6/uPAR-related protein 1, pro-epidermal growth factor precursor (pro-EGF protein), and CD59 glycoprotein; and a suitable container means. In some embodiments, said protein is selected from the group consisting of: (a) apolipoprotein A-IV, thymosin β4, insulin-like growth factor-binding protein 1, vitamin D binding protein, profilin-1, glutathione peroxidase 3, superoxide dismutase [Cu—Zn], neutrophil defensin 1, and (b) non-secretory ribonuclease, secreted Ly-6/uPAR-related protein 1, pro-epidermal growth factor precursor (pro-EGF protein), and CD59 glycoprotein. The protein may be angiotensinogen. The antibody may be conjugated to a label, such as a fluorophore or an enzyme. The antibody may be comprised in a lateral flow device. The kit may further comprising an additional 1, 2, 3, 4, 5, 6, 7, 8, 9, 10 or more antibody or antibodies for measuring an additional protein from group (a) or group (b). The kit may further comprise antibodies for measuring all proteins from group (a) and group (b). The kit may determine or provide instructions for calculating a ratio or relationship between proteins in group (a) and group (b). The kit may further comprise a package insert providing instructions for measuring the expression levels of the markers in a biological sample from the individual and/or determining the risk or likelihood of developing a nephropathy or kidney disease. The kit may be a point of care kit, such as, e.g., a dip-stick for assessing the concentration of said protein. The kit may further comprise instructions for determining the likelihood of developing a progressing or worsening acute kidney injury in the subject.

In various aspects, one or more of the biomarkers in Table 1A or Table 2 may be used to detect or predict the onset, progression, or severity of a kidney disease such as AKI in a subject. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more of the biomarkers in Table 1A or Table 2 may be used. In some embodiments, 1, 2, 3, 4, 5, 6, 7, 8, 9, 10, 11, 12, 13, 14, or 15 of the biomarkers in FIG. 8 may be used. Table 1A shows the expression of all 344 proteins in patients with AKI after cardiac surgery who did or did not require dialysis. FIG. 8 shows the expression of candidate proteins in 3 proteomic experiments. FIG. 10 shows that urinary angiotensinogen increases in patients with diabetes and normal renal function, who will later have a loss of renal function.

As used herein, “obtaining a biological sample” or “obtaining a urine sample” refer to receiving a biological or urine sample, e.g., either directly or indirectly. For example, in some embodiments, the biological sample, such as a urine sample, is directly obtained from a subject at or near the laboratory or location where the biological sample will be analyzed. In other embodiments, the biological sample may be drawn or taken by a third party and then transferred, e.g., to a separate entity or location for analysis. In other embodiments, the sample may be obtained and tested in the same location using a point-of care test. In these embodiments, said obtaining refers to receiving the sample, e.g., from the patient, from a laboratory, from a doctor's office, from the mail, courier, or post office, etc. In some further aspects, the method may further comprise reporting the determination to the subject, a health care payer, an attending clinician, a pharmacist, a pharmacy benefits manager, a researcher, a pharmaceutical company, or any person that the determination may be of interest.

The term “reference level”, as used herein, refers to a control level or threshold value that is associated with a range present in a healthy or control sample. For example, urinary angiotensinogen may be measured in a test sample and then compared to a control sample or a reference value, such as a cutoff value or a threshold value (e.g., a concentration level, where values below the concentration level are not associated with an increased risk of a kidney disease or nephropathy). The reference value may be provided in materials in a kit. In some embodiments, one or more control samples may be used to generate a reference level. The reference level may be different for populations or subjects with different clinical conditions, medications or demographic characteristics. For example, a reference level may be different for children than for adults. As another example, the reference level may be different for subjects with chronic kidney disease than for subjects with normal baseline kidney function. As another example, the reference level may be different for rats than for humans.

Although, in certain embodiments, human subjects may be tested for the presence or an increased risk of a nephropathy or kidney disease, such as a progressive or worsening nephropathy or kidney disease, it is anticipated that the methods may be used to test a non-human mammal, such as a dog, cat, horse, sheep, rabbit, pig, rat, mouse, or non-human primate, or non-mammalian subject such as a zebrafish.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The words “acute kidney injury”, “AKI”, “kidney injury” and “kidney failure” refer to injury or damage to the kidney which may be reversible or irreversible. Many definitions of acute kidney injury have been used in the literature. Most definitions refer to specific values of increased serum creatinine or decreased volumes of urine output. The use of these terms in this document does not intend to be constrained by definitions which require changes in serum creatinine or urine output. The use of the terms is also not constrained by the time over which injury occurs. The use of these terms may reflect injury or damage to any region of the renal nephron or kidney parenchyma.

As used in this specification and claim(s), the words “diabetic kidney disease” and “diabetic nephropathy” may refer to the development of proteinuria and/or albuminuria and/or the loss of renal function at a greater than expected rate and/or to developing a GFR or an estimated GFR less than about 60 ml/minute. Loss of renal function may occur with or without proteinuria or albuminuria.

As used in this specification and claim(s) the words “dialysis” and “end stage renal disease” may include the initiation of hemodialysis, peritoneal dialysis, or transplantation. End stage renal disease may result in death from renal failure.

The word “risk” may refer to the chance or probability or odds ratio that a subject will experience an outcome such as, e.g. AKI, worsening AKI, severe AKI, renal replacement therapy, death, cardiovascular death, diabetic kidney disease, worsening diabetic kidney disease, chronic kidney disease, worsening chronic kidney disease, end stage renal disease, improving kidney function, or recovery from kidney injury.

Chronic kidney disease can be a risk factor for death from other causes, such as heart disease, and worsening kidney disease can increase the risk of death, e.g., from cardiovascular complications. Therefore, urine biomarker protein concentration(s) may be used, e.g., as a marker for the development of cardiovascular death or death from any cause. Urine biomarker protein concentration(s) may be used as a marker for the development of either diabetic nephropathy and/or acute kidney injury (AKI). Although, in some embodiments, a single biomarker protein may consist of a single protein, a combination of proteins or a ratio of proteins may be used which are predictive of a specified outcomes. The combination or ratio of a listed protein with a protein which is not listed among the claimed protein biomarkers may be used. The combination or ratio of proteins may include 2, 3, 4, 5, 6, 7, 8 or more proteins.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C. Proteins identified by LC-MS/MS in the urine of patients with AKI following cardiac surgery. (FIG. 1A) The Venn diagram shows the number of proteins identified in patients who developed severe post-operative AKI versus those who only developed mild AKI. (FIG. 1B) The volcano plot shows the significance of the difference in the abundance between the two groups for each identified protein. It allows for the selection of candidate biomarkers that have a large magnitude fold change (positive fold changes indicate elevated protein levels in the RRT group) and highly significant p-values (from Wilcoxon Rank-Sum test). The arrowhead indicates the data point for angiotensinogen. Proteins above the dashed line had a p-value<0.05. (FIG. 1C) The scattergram shows the angiotensinogen level in each patient by group. Angiotensinogen was only identified in the urine of 4 of 6 AKI patients in the No RRT group. The line shows the threshold at which there is no overlap between the two groups.

FIGS. 2A-B. (FIG. 2A) Box and whisker plots showing the distribution of creatinine corrected angiotensinogen values (ng of urine angiotensinogen/mg of urine creatinine) by group in patients who developed AKI within 48 hours after cardiac surgery. uAnCr increased in a graded manner with AKI severity (as determined by maximum AKIN Stage) in patients who developed AKI within 48 hours after cardiac surgery (n=97). uAnCr increased in a graded manner with AKI severity in the subset of patients who were classified as AKIN Stage 1 AKI at the time that their urine samples were collected (n=79). Box plots show the median (solid line), 25th and 75th percentiles. Error bars represent the 5th and 95th percentiles. AKIN Stage groups were compared with the Kruskal-Wallis test (p-value shown in bottom right). The RRT group was not used in this analysis because it represents a subset of the AKIN Stage 3 group. The * represents a p-value<0.05 compared to AKIN Stage 1 in the post-hoc Dunn's test for pairwise comparison. (FIG. 2B) ROC curves show the predictive power of uAnCR for multiple outcomes in patients who develop AKI within 48 hours after cardiac surgery (n=97). For each tested outcome, cut-offs are iteratively determined throughout the dataset, and the sensitivity (true positive rate) and 1-specificity (false positive rate) is calculated at each cut-off. These values are plotted against each other, and the area under the resulting ROC curve (AUC) is used to evaluate the predictive power of the biomarker. A perfect biomarker would have an AUC of 1.0, whereas random chance (having equal true positive and false positive rates) would have an AUC of 0.5 (shown as gray diagonal line).

FIG. 3. Higher levels of creatinine corrected urinary angiotensinogen are related to longer stay in the hospital. Survival curves showing the differences in the time to discharge (days after sample collection) in patients with different concentrations of urinary angiotensinogen. Cardiac surgery patients were ranked into tertiles based upon their post-operative urinary angiotensinogen concentrations. The time to discharge (day 0 is the day of collection) for each group was then modeled using a Kaplan-Meier survival analysis.

FIGS. 4A-B. Distribution of urinary angiotensinogen by group in off pump cardiac surgery patients and corresponding ROC curves for post-operative outcomes. (FIG. 4A) Scatterogram showing the creatinine corrected urinary angiotensinogen concentration of each patient by group. The * indicates a p<0.05 compared to Pre-Op, and the # indicates p<0.05 compared to No AKI. The overall p-value of the ANOVA on Ranks test was 0.003. (FIG. 4B) ROC curves show the predictive power of creatinine corrected urinary angiotensinogen with respect to outcomes related to worsening renal function and AKI severity.

FIGS. 5A-B. Urinary angiotensinogen levels predict outcomes in AKI patients in the ICU. (FIG. 5A) Box plots show the differences in the median and interquartile range of creatinine corrected urinary angiotensinogen in the ICU who did or did not meet the composite outcome of RRT of death (defined as in-hospital mortality). Error bars represent the 95^{th} and 5^{th} percentiles. The dashed lines indicate the mean value for each group. (FIG. 5B) ROC curves show that urinary angiotensinogen is able to predict the composite outcomes RRT or death and AKIN stage 3 AKI or death.

FIGS. 6A-B. Higher levels of creatinine corrected urinary angiotensinogen are related to longer length of stay (LOS) in the hospital. (FIG. 6A) Survival curve showing the differences in the time to discharge (days after sample collection) in the ICU patients with different concentrations of urinary angiotensinogen. Patients were ranked and classified in tertiles based on their creatinine corrected angiotensinogen values. The time to discharge (day 0 is the day of collection) for each group was then modeled using a Kaplan-Meier survival analysis. Patients who died were in included but were censored (dots). (FIG. 6B) ROC curve showing the ability of creatinine corrected angiotensinogen to predict decreased LOS (defined as <7 days after sample collection).

FIG. 7. ROC curve analysis shows that urinary angiotensinogen can predict the need for renal replacement therapy in AKI patients of diverse etiologies. This analysis included patients who had AKI during their stay in the ICU or following cardiac surgery without intraoperative cardiopulmonary bypass (n=68). Sixteen patients required RRT.

FIG. 8. Urine protein changes in three proteomic studies to identify biomarkers that can predict the onset or severity of acute kidney injury. White bars show mean protein expression in the early AKI study in which protein expression from urine of patients who had not yet developed AKI after cardiac surgery was determined.—indicates patients that did not later develop AKI and + indicates patients that did develop AKI. Dark grey bars show urine protein expression in patients who had developed mild AKI at the time urine was collected.—indicates urine from patients that did not later develop severe AKI requiring renal replacement therapy and +indicates patients that did develop severe AKI requiring renal replacement therapy. Light grey bars show urine protein expression in rats that were administered glycerol to cause acute kidney injury.—indicates urine from control (saline) rats and +indicates glycerol (AKI) rats. Fifteen candidate markers were identified which increase in AKI and six candidates that decrease during AKI. The three bars on the left side of each panel are the no AKI (EARLY study and RAT study) or no RRT groups. White, EARLY; Dark Grey, RRT; Light Grey, RAT.

FIGS. 9A-E. Total ion chromatogram of digested urine proteins. (FIG. 9A) Black arrow indicates time at which the haptoglobin beta chain tryptic peptide elutes off column. (FIG. 9B) Extracted ion chromatogram for the endogenous haptoglobin beta chain tryptic peptide VTSIQDWVQK (602.3 m/z, +2 charge) and the stable isotope internal standard VTSIQDWVQK* (606.3 m/z, +2 charge). (FIG. 9C) Fragment ions for parent masses 602.3 and 606.3 m/z. Fragment y ions 803.4 and 811.4 (indicated by circles above) were selected as examples for downstream quantification. (FIG. 9D) Extracted ion chromatograms for fragment y ions 803.4 and 811.4. Area under the curve is used to estimate quantity from an external standard curve (FIG. 9E) constructed using a synthetic peptide. Observed differences from the expected concentration of the internal standard can be used to estimate total losses, matrix effects, and differences in digest efficacy and is applied to the calculation of the endogenous tryptic peptide. Monitoring several fragment ions from several tryptic peptides provides an estimate of endogenous protein quantity.

FIG. 10. Angiotensinogen in diabetes. Results are shown for 7 candidate biomarkers to predict the development of diabetic nephropathy over the subsequent 6 years in patients. Urine proteins were measured by multiple reaction monitoring. Stable. Patients that had less than 50% increase in serum creatinine over 6 years of follow-up. Decline- Patients that had an increase in creatinine of at least 60%. The p value is for the analysis that the AUC is different than 0.5. The concentration of angiotensinogen was statistically higher in patients that later had a decline in renal function. The area under the ROC curve for angiotensinogen to predict the decline in renal function was 0.71 with a p value that did not quite meet statistical significance when compared to an AUC value of 0.5 in this small set.

FIG. 11. Sequence coverage of angiotensinogen (SEQ ID NO:1) identified by LC-MS/MS 7 unique peptides, 63/485 amino acids (13% sequence coverage) Identified sequences are shaded. Renin cleaves angiotensinogen at amino acid 43 (denoted by asterisk), to release angiotensin I.

FIG. 12. Mass spectrum of representative angiotensinogen peptide with a parent ion mass of 1267.75 AMU (ALQDQLVLVAAK) (SEQ ID NO:19) obtained from human urine of a patient who developed acute kidney injury after cardiac surgery and later required renal replacement therapy.

FIG. 13. Sequence coverage of Pro-epidermal growth factor from the RRT study. The signal peptide is the 22 amino acid sequence at the beginning of the protein marked with an overline. The sequence that is cleaved to form epidermal growth factor is shown in the box. The peptides from within the sequence that were identified are shown in grey highlighting. The region of the protein that serve as a biomarker for AKI are not the amino acid sequence that codes for the epidermal growth factor protein.

### DESCRIPTION OF ILLUSTRATIVE EMBODIMENTS

The present invention provides, in various aspects, biomarkers of kidney disease such as AKI. In various embodiments, one or more biomarkers of AKI that can predict which patients will likely develop severe disease at the time of diagnosis may be used to facilitate timely intervention, e.g., in a high risk population. In some aspects, urinary protein biomarkers of the present invention may be tested to determine if a test compound or experimental or approved drug exhibits renal toxicity in a subject, such as a mammal, mouse, rat, rabbit, pig, dog, zebrafish, primate, monkey, chimpanzee, or human. In other embodiments, a urine sample may be obtained from a patient, e.g., after a cardiac surgery or other potentially renal injuring occurrence, to determine if the patient has or will likely develop AKI, worsening AKI, or other kidney disease (e.g., a chronic kidney disease, a rapidly progressing kidney disease, or an end-stage renal disease).

As described in the below examples, liquid chromatography-tandem mass spectrometry was used to identify the 30 prognostic urinary proteins listed in Table 2 as biomarkers of severe AKI in a group of patients that developed AKI after cardiac surgery. Of the biomarkers listed in Table 2, angiotensinogen had the best discriminative characteristics. Urinary angiotensinogen was subsequently measured by ELISA and its prognostic predictive power in 97 patients who developed AKI after cardiac surgery was verified. The urine angiotensinogen-to-creatinine ratio (uAnCR) predicted the following outcomes: discharge≦7 days from sample collection, worsening of AKI, AKIN stage 3, the need for renal replacement therapy (RRT), and the composite outcomes AKIN stage 2 or 3, AKIN stage 3 or death, and RRT or death. The prognostic predictive power of uAnCR was improved when only patients classified as AKIN stage 1 at the time of urine sample collection (n=79) were used in the analysis, among whom it predicted development of AKIN stage 3 or death with an AUC value of 0.81. Finally, the inventor found that the prognostic predictive power of uAnCR was augmented in patients who underwent off-pump cardiac surgery (n=22), in whom it was an excellent predictor of AKIN stage 3, and RRT (AUC=0.93 and 0.86, respectively). These data demonstrate the potential utility of angiotensinogen as a prognostic biomarker of AKI, e.g., after cardiac surgery.

Cardiac surgery is an excellent setting in which to identify novel prognostic biomarkers of AKI. Approximately 20% of patients who undergo cardiac surgery develop AKI as a post-operative complication, and importantly, both the timing and the severity of the injury can be readily determined in these patients (Englberger et al., 2011). Additionally, because AKI after cardiac surgery has a complex pathophysiology involving ischemic injury, nephrotoxicity, and inflammation, biomarkers discovered in this setting may be applicable to AKI of other causes as well (Rosner et al., 2008). In this study the inventors used liquid chromatography-tandem mass spectrometry (LC-MS/MS) to analyze samples obtained from four Southern Acute Kidney Injury network (SAKInet) institutions to identify candidate prognostic urinary biomarkers of AKI following cardiac surgery. The inventors subsequently performed an initial verification of one of these biomarkers, angiotensinogen, in a larger set of samples from patients who developed AKI following cardiac surgery. This is the first study to demonstrate the potential clinical utility of angiotensinogen as a prognostic biomarker of AKI, and it supports a growing body of literature suggesting a role for the renin-angiotensin system in the pathobiology of AKI.

As shown in the below examples, the inventors used urinary proteomics to identify several candidate biomarkers for the prediction of the development of severe AKI. The inventors then verified the biomarker capability of the most promising candidate, angiotensinogen, in a larger set of cardiac surgery patients using a commercially available ELISA assay. Urinary angiotensinogen was corrected for urine creatinine (uAnCR) in an attempt to control for biological variability in urine concentration. The inventors found that uAnCR increased with AKI severity, and it was predictive of the relevant outcomes including: worsening of AKI, development of AKIN stage 3, need for RRT, length of stay, as well as the composite outcomes AKIN stage 2 or 3, AKIN stage 3 or death, and need for RRT or death. Furthermore, the prognostic predictive power was improved when only patients who had AKIN stage 1 at the time of sample collection were used in the analysis. The analysis of this subpopulation allowed the inventors to determine the ability of the biomarker to predict adverse outcomes among patients that had not yet developed severe AKI as measured by serum creatinine, and it demonstrates the ability of urinary angiotensinogen to predict severe AKI and adverse outcomes at an early stage in the disease course. While it remains to be seen if this would improve the outcomes of these patients, it suggests that angiotensinogen (alone or in combination with other biomarkers) could be useful in the design of clinical trials by facilitating the identification of high risk patients in whom to test an intervention. Finally, the inventors found that the predictive power of angiotensinogen was substantially improved in patients who had undergone off-pump cardiac surgery. Without wishing to be bound by any theory, this could indicate that bypass itself increases urinary angiotensinogen.

In spite of the potential confounding effect of cardiopulmonary bypass on urinary angiotensinogen, uAnCR was a strong predictor of adverse outcomes in the entire group. However, its exceptional predictive power for severe adverse outcomes in off-pump patients suggests that it could also have prognostic value in patients undergoing other major surgeries in the thoracic and abdominal cavities, which have been recognized as a common precipitating factor of AKI, or in AKI in other non surgical settings. However, it will be necessary to confirm these findings in larger studies specifically designed to evaluate AKI in settings that do not involve intraoperative cardiopulmonary bypass. In total, these data demonstrate the potential of angiotensinogen as prognostic biomarker of AKI. While the inventors did not directly compare its prognostic predictive power to that of other biomarkers, the results are at least comparable to what has been reported in the literature for previously described AKI biomarkers. For example, Hall et al. (2011) reported unadjusted AUCs of 0.71, 0.64, and 0.63 for the prediction of the composite outcome of worsening of AKI or death for urine NGAL, KIM-1 and IL-18, respectively. Koyner et al. (2012) recently reported unadjusted AUCs of 0.58, 0.63 and 0.74 for urine NGAL, urine IL-18, and plasma NGAL, respectively, for the outcome of worsening of AKI (Hall et al., 2011; Koyner et al., 2012). Thus, the combination of uAnCR with these biomarkers could improve risk reclassification models in these patients.

Without wishing to be bound by any theory, the identification of urinary angiotensinogen as a novel AKI biomarker may improve understanding of the pathobiology of this disease. Angiotensinogen is the principal substrate of the renin-angiotensin system (RAS), a hormonal cascade that has pleitropic effects in the kidney, including the regulation of hemodynamics, sodium reabsorption, aquaresis, cellular proliferation and apoptosis, fibrosis, and inflammation (Velez, 2009). It has been implicated in several nephropathologies, including diabetic nephropathy (Brenner et al., 2001; Lewis et al., 1993). Additionally, it is crucial for proper nephrogenesis (Kim et al., 1995). The data disclosed herein suggests that it could be involved in either renal injury or recovery from injury during AKI. This is supported by a number of observational studies that have noted an association between pharmacologic inhibition of the renin-angiotensin system and AKI risk, although it is noteworthy that there are conflicting reports in the literature (Arora et al., 2008; Benedetto et al., 2008; Plataki et al., 2011; Yoo et al., 2010). Furthermore, the ACE II genotype has been associated with increased risk of AKI in the ICU (de Cheyron et al., 2008). It is unclear whether the elevated levels of urinary angiotensinogen observed in severe AKI reflect an activation of the RAS vis-à-vis cleavage of existing angiotensinogen into angiotensin 1 and subsequent bioactive molecules in the RAS hormonal cascade. Interestingly, the identified portions of angiotensinogen in the below proteomics study did not identify the proximal domain of angiotensinogen from which angiotensin 1 is cleaved by renin Likewise, the monoclonal antibody used to quantify angiotensinogen in the ELISA the inventors used recognizes an epitope distal to the angiotensin 1 domain, and so it is predicted to be insensitive to the detection of proteolytic cleavage of angiotensinogen by renin. Also unclear at this point is whether increases in angiotensinogen are systemic or intrarenal in nature. However, others have shown that the intrarenal RAS is activated following renal ischemia-reperfusion injury in a rat model (Allred et al., 2000).

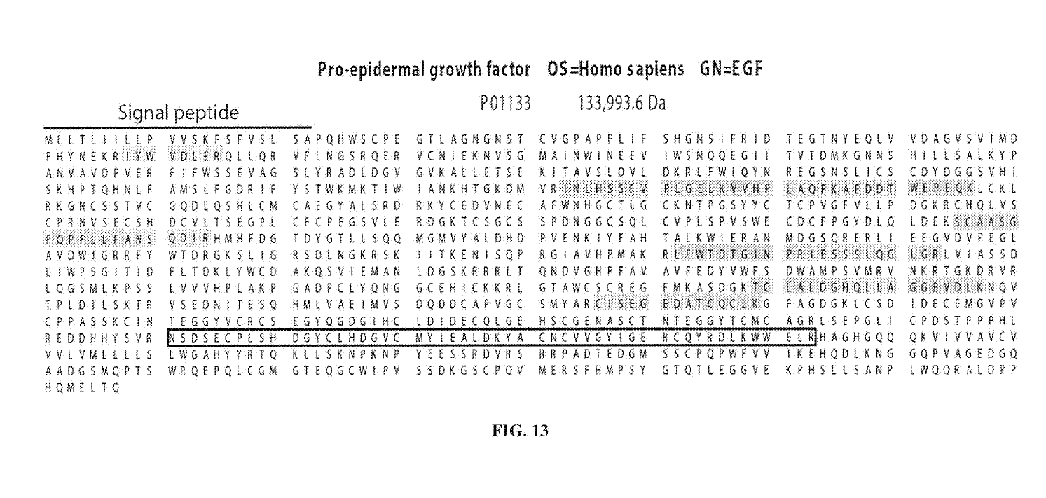

Epidermal growth factor was found by the inventors to decrease in AKI. It is a 53 amino acid protein involved in stimulation of growth of tissues. The biologically active portion of the protein is produced by cleavage of amino acid residues 971-1023 from the pro-epidermal growth factor precursor protein (Bell et al., 1986). EGF is highly expressed in normal kidneys and the expression of EGF decreases with tubular damage in transplanted kidneys (Di Paolo et al., 1997). Administration of exogenous EGF accelerates repair of renal tubules after ischemia reperfusion injury in rats (Humes et al., 1989). Urinary levels of EGF decrease in acute kidney injury (Askenazi et al., 2012). Although levels of the epidermal growth factor hormone can decrease in AKI, to the knowledge of the inventors, changes in the region of the pro epidermal growth factor precursor that are located proximal to the active hormone in the precursor molecule have not been previously associated with AKI. The portion of the pro-EGF protein has been identified herein as a candidate biomarker that decreases in urine of patients with acute kidney injury (FIG. 13).

Apolipoprotein A-IV. There is no previous evidence for increases in this protein during AKI but there is evidence in chronic kidney disease (CKD) and in transplant rejection. It is increased in serum in the early stages of CKD and the increases in serum are associated with progression of CKD. Immunohistochemistry shows APO A-IV in brush border of proximal tubule and also in distal tubules. No renal mRNA was seen demonstrating that it is not synthesized in the kidney but is reabsorbed. Apo A-IV increased in kidney tissue at 7 days in patients with acute allograft rejection. Increases in the urine during AKI may reflect proximal tubular injury causing reduced reabsorption. Apolipoprotein A-IV has the gene name APOA4 and is also referred to as Apolipoprotein A4, Apo-AIV, and/or ApoA-IV. The uniprot identifier for the human form is P06727. Generally, Apo A-IV is synthesized in the intestine, and it has shown that Apo A-IV is present in urine and in human kidney. Immunohistochemistry has observed APO A-IV in brush border of proximal tubule and also in distal tubules. No renal mRNA was seen, demonstrating that it is not synthesized in the kidney but is reabsorbed. Loss or decreases of Apo A-IV may be associated with nephrotic syndrome. Apo A-IV may be increased in serum in the early stages of CKD, and serum concentrations of Apo A-IV may be used as a predictor of progression of chronic kidney disease, wherein patients with higher serum levels of Apo A-IV can indicate a more rapid progression of CKD. As shown in the below examples, Apo A-IV has been identified as a biomarker of AKI. The mechanism by which Apo A-IV could act as a biomarker is presently unclear. Without wishing to be bound by any theory, one possibility is that Apo A-IV may be filtered and taken up by the megalin-cubilin complex in the proximal tubule. Thus, injury to the proximal tubule during AKI could thus be reflected by decreased reabsorption and increased urinary concentrations of Apo A-IV.

Pigment epithelium-derived factor (PEDF) was first identified as a protein from retinal pigment epithelial cell conditioned medium which induces differentiation of cultured neural cells. Changes in PEDF have not been described in AKI but it does increase in the urine of patients with diabetes and acute allograft rejection.

Thymosin beta 4 plays an important role in cytoskeletal reorganization by binding to G actin to inhibit actin polymerization. It is also angiogenic. Administration of thymosin beta 4 promotes wound healing. Message for thymosin beta-4 is increased early after renal ischemia reperfusion injury in rats. In a 5/6 nephrectomy model for glomerulosclerosis, thymosin beta 4 was increased in sclerotic glomeruli. Thymosin beta 4 was necessary in cultured glomerular endothelial cells for angiotensin II induced pai-1 expression. These data suggest that thymosin beta 4 increases may be partially responsible for fibrosis in glomerulosclerosis. Measurement of this protein could lead to a marker that would predict long term outcomes in the interaction between AKI and CKD. Thymosin beta 4 has the gene name TMSBX4 and the synonymous gene names TBX4, THYB4 and TMSB4. The protein has the alternative names T beta 4 and Fx. It can be cleaved into hematopoietic system regulatory peptide which is also called seraspenide. Generally, it can play an important role in cytoskeletal reorganization by binding to G actin to inhibit actin polymerization. It can also exhibit angiogenic properties. Administration of thymosin beta 4 may promote wound healing. Thymosin beta-4 may be increased early after renal ischemia reperfusion injury in rats. In a 5/6 nephrectomy model for glomerulosclerosis, thymosin beta 4 was increased in sclerotic glomeruli. Thymosin beta 4 was necessary in cultured glomerular endothelial cells for angiotensin II induced pai-1 expression. Without wishing to be bound by any theory, these data support the idea that thymosin beta 4 increases may be partially responsible for fibrosis in glomerulosclerosis. Measurement of this protein may be used to predict longer term outcomes in the interaction between AKI and CKD.

Insulin-like growth factor-binding protein 1 binds to insulin-like growth factor leading to a prolongation of its half-life and changing its biological action. It enhances cell proliferation but has also been reported to decrease IGF bioactivity. Message for IGFBP1 increases following HgC12 induce AKI and folic acid-induced AKI. In a radiocontrast model of AKI, mRNA for IGFBP1 increases within two hours in both the cortex and medulla. In an analysis of patients in the PICARD study serum levels of IGFBP1 trended toward being higher in the group of patients with AKI and diabetes that did not survive (p=0.056). These data suggest that IGFBP1 may be involved in the recovery from AKI. Insulin-like growth factor-binding protein 1 has the gene name IGFBP1 and the gene name synonym IBP1. Other names for this protein are IBP-1, IGF-binding protein 1, IGFBP-1 and placental protein 12. Generally, IGFBP1 binds to insulin-like growth factor leading to a prolongation of its half-life and changing its biological action. It can enhance cell proliferation, but may also decrease IGF bioactivity. Increases in IGFBP1 were observed in models of AKI (e.g., following HgCl₂ induced AKI and folic acid-induced AKI). In a radiocontrast model of AKI, mRNA for IGFBP1 was observed to increase within two hours in both the cortex and medulla. In an analysis of patients in the PICARD study, serum levels of IGFBP1 trended toward being higher in the group of patients with AKI and diabetes that did not survive (p=0.056). Without wishing to be bound by any theory, these data support the idea that IGFBP1 may be involved in recovery from AKI.

Myoglobin. Myoglobin is expressed in cardiac and skeletal myocytes and is released following injury to these cells. Higher serum myoglobin levels have been observed in patients with AKI after cardiac surgery compared to controls and high levels were associated with the need for RRT.

Vitamin D binding protein. This protein is expressed in the liver, although numerous cell types can produce it. It has many functions, including binding Vitamin D and free actin, preventing its polymerization. It is involved in the response to injury, both as an actin scavenger and as an immunomodulator. Notably it stimulates apoptosis in macrophages, but also is a neutrophil chemoattractant (via enhancement of C5a). No evidence linking Vitamin D binding protein with AKI has been published. Vitamin D binding protein has the gene name GC. It is also referred to as Gc-globulin, Group-specific component, DBP and VDB. The human form has the uniprot identifier P02774. Generally, this protein is expressed in the liver, although numerous cell types can produce it. It has many functions, including binding Vitamin D and free actin, preventing its polymerization. It can be involved in the response to injury, both as an actin scavenger and as an immunomodulator. Vitamin D binding protein may stimulate apoptosis in macrophages, but may also act as a neutrophil chemoattractant (e.g., via enhancement of C5a). Vitamin D binding protein may be lost in the urine in glomerular diseases, and the urinary loss may be attenuated by use of ACE inhibitors. As shown in the below examples, vitamin D binding protein may be used to identify or predict AKI.

Complement C4-B. This protein is part of the classical complement cascade but can also be activated by the mannose binding lectin pathway. It is one of several complement cascade proteins the inventors observed to increase in the urine during AKI.

Profilin-1. To the knowledge of the inventors, this protein has not been previously associated with AKI. It promotes actin polymerization at low concentrations. Actin polymerization occurs in tubular injury. Polymerization in AKI may be partially mediated by the increase in profilin-1. Profilin-1 has the gene name PFN1, and alternative names include epididymis tissue protein Li 184a and profilin I. As shown in the below examples, profilin-1 can be associated with AKI. Profilin I can promote actin polymerization at low concentrations, and actin polymerization may occur in tubular injury. Without wishing to be bound by any theory, polymerization in AKI may be partially mediated by the increase in profilin-1.

Glutathione peroxidase 3. This protein protects tissues from oxidative stress. In cultured renal tubule cells (mIMCD3), hydrogen peroxide lead to an increased expression of message for GPx3. c-maf may be the transcriptional factor responsible for the increase. There is no previous evidence for a role in AKI. Glutathione peroxidase 3 has the gene name GPX3 and the gene synonym GPXP. This protein has the alternative names extracellular glutathione peroxidase and plasma glutathione peroxidase. Short names for this protein are GPx-3 and GSHPx-3. Glutathione peroxidase 3 can protect tissues from oxidative stress. In cultured renal tubule cells (mIMCD3), hydrogen peroxide lead to an increased expression of message for GPx3. Without wishing to be bound by any theory, it is envisioned that c-maf may be the transcriptional factor responsible for the increase. As shown in the below examples, glutathione peroxidase 3 can be used as a biomarker to identify or predict AKI.

Superoxide dismutase [Cu—Zn] protects against oxidative stress. It is located in tubules of human kidney. Unilateral renal artery stenosis decreased sod1 protein. Administration of adenovirus containing the gene for sod1 reduced the magnitude of I/R AKI and cyclosporine nephrotoxicity in rats. I/R AKI is worse in SOD1 deficient mice. In contrast to the inventors' findings of an increase in sod1 in AKI, both mRNA and sod1 protein decreased in the kidney of rats with endotoxemia which induced AKI. Superoxide dismutase [Cu—Zn] protects against oxidative stress. It has the gene name SOD1 and, the alternative name Superoxide dismutase 1 and the uniprot identifier for the human form is P00441. It may be observed in tubules of human kidney. In contrast to findings presented in the below examples, both mRNA and sod1 protein were observed to decrease in the kidney of rats with endotoxemia which can induce AKI (Leach et al., 1998). In contrast and as shown in the below examples, an increase in sod1 may be used to identify or predict AKI.

Complement C3. This is another complement cascade protein that is increased in AKI. Activation of the alternative pathway can occur by deposition of C3 on tubular epithelial cells following injury when the complement inhibitor Crry is redistributed away from the basolateral surface in injury.

Antithrombin III mRNA is found in the kidney of rats. In humans, ATIII is localized to vesicle-like structures in proximal tubular cells suggesting that filtered ATIII is reabsorbed. Thus tubular injury could cause increased urinary levels. ATIII promotes the release of PGI2 from endothelial cells in vivo which can inhibit leukocyte activation. In intestinal ischemia reperfusion injury treatment with ATIII reduces neutrophil adhesion and vascular permeability. There is no previous evidence for changes in renal or urinary ATII levels in AKI. However, pretreatment with ATIII ameliorates the increases in SCr and renal malondialdehyde and myeloperoxidase levels and reduces the histological evidence of injury in a renal ischemia reperfusion model. ATIII was dramatically increased in the inventors Early AKI proteomic study and may be a good early marker of injury.

Neutrophil defensin 1. Alpha defensins are expressed primarily in neutrophils and have antibacterial activity. It also activates a number of immunologic cell types and proinflammatory cytokines. The increase in neutrophil defensin 1 that the inventors observed may be due to release from neutrophils that have migrated into the injured kidney. However, defensins also have anti-inflammatory and other effects that could promote recovery from AKI. They inhibit the activation of the classical complement cascade and promote mitogenesis of epithelial cells. Thus the defensins could have both beneficial and detrimental effects on the development and recovery from AKI. Release of defensins could be an early indicator of renal injury as suggested by the large increase seen in the inventors' early markers study. There has not been any previous identification of neutrophil defensin 1 as a urinary biomarker of AKI. Neutrophil defensin 1 has the gene name DEFA1 and DEFA1B. Synonymous gene names are DEF1, DEFA2 and MRS. Alternative names for the protein are Defensin, alpha 1 and HNP-1. Short names for the protein are HP-1 and HP-2. The protein can be cleaved into HP 1-56 and neutrophil defensin 2. Generally, alpha defensins are expressed primarily in neutrophils and can exhibit antibacterial activity. Without wishing to be bound by any theory, increases in neutrophil defensin 1 may at least in part be due to release from neutrophils that have migrated into an injured kidney. Alternately, defensins also have anti-inflammatory and other effects that might promote recovery from AKI. They may inhibit the activation of the classical complement cascade and promote mitogenesis of epithelial cells. Thus defensins might have both beneficial and detrimental effects on the development and recovery from AKI. Release of defensins may be used as an early indicator of renal injury, as supported by the large increases observed in the early markers studies included in the below examples. As shown in the below examples, neutrophil defensin 1 may be used as a urinary biomarker of AKI.

Lysozyme C is produced primarily by macrophages and is involved in the innate immune response. Lys C has been used as an index of renal injury in a rat model of nephrotoxicity. Lysozyme has been implicated in sepsis-induced AKI, and may itself be a nephrotoxin.

Non-secretory ribonuclease was found by the inventors to decrease in AKI. During AKI urine microvesicles cause proliferation and inhibit apoptosis which may be protective during AKI. The proliferative effect is inhibited by the addition of RNAse. Thus a decrease in RNAse activity during AKI may promote proliferation and recovery. There is no previous data for changes in the concentration of this protein in the urine during AKI. Non-secretory ribonuclease has the gene name RNASE2 and the gene name synonyms EDN and RNS2. This protein is also called eosinophil-derived neurotoxin, RNase Upl-2, and ribonuclease 2. As shown in the below examples, It was found by the inventors to decrease in AKI. During AKI urine microvesicles can cause proliferation and inhibit apoptosis, which may be protective during AKI. The proliferative effect may be inhibited by the addition of RNAse. Thus, a decrease in RNAse activity during AKI may promote proliferation and recovery. As shown in the below examples, changes in the concentration of this protein in the urine were associated with AKI.

Secreted Ly-6/uPAR-related protein 1 was found by the inventors to decrease in AKI. It is expressed in keratinocytes but has also been isolated from urine. It inhibits angiogenesis in Kaposi's sarcoma and inhibited proliferation in endothelial cell lines. Decrease in this protein may aid in proliferation of regenerating tubules. There are no previous data for changes during AKI. Secreted Ly-6/uPAR-related protein 1 has the gene name SLURP1 and alternative protein names of ARS component B, ARS(component B)-81/S, and anti-neoplastic urinary protein. As shown in the below examples, it was observed to decrease in AKI. Generally, this protein is expressed in keratinocytes, and may be isolated from urine. This protein may inhibit angiogenesis in Kaposi's sarcoma, and it may inhibit proliferation in endothelial cell lines. Without wishing to be bound by any theory, it is envisioned that decrease in this protein may aid in proliferation of regenerating tubules.

Uromodulin was found by the inventors to decrease in AKI. It is expressed in the thick ascending limb. It is renoprotective in an ischemia-reperfusion model, and this has been attributed to its anti-inflammatory effects, specifically by altering the expression of TLR4 and MIP-2. It has been shown to translocate from the apical membrane to the basolateral membrane during tubular injury. This could decrease its shedding/secretion into the urine in AKI.

Polymeric IgG receptor was found by the inventors to decrease in AKI. It is involved in the secretion of soluble IgA. It is expressed primarily in the TAL and DCT. It has been previously demonstrated that levels of the secretory component of this protein decrease in the urine following renal IRI, in agreement with the inventors' studies. Lower levels of this normally expressed protein could indicate distal tubular dysfunction seen in AKI.

CD59 glycoprotein was found by the inventors to decrease in AKI. CD59 is anti-inflammatory, binding and neutralizing the membrane attack complex. Therefore, loss of CD59 could lead to increased inflammatory injury. The inventors saw a decrease in urinary CD59 in both studies. However, there is no evidence that loss of CD59 alone can exacerbate AKI, although loss of both CD55 and CD59 has been shown to do so in a rat model. CD59 glycoprotein has the gene name CD59 and the alternative gene names MIC11, MIN1, MIN2, MIN3 and MSK21. It has alternative protein names of 1F5 antigen, 20 KDa homologous restriction factor, MAC-inhibitory protein, MEM43 antigen, Membrane attach complex inhibition factor, membrane inhibitor of reactive lysis, protectin and CD_antigen=CD59. As shown in the below examples, it has been observed to decrease in AKI. It can act as an anti-inflammatory, binding and neutralizing the membrane attack complex. Without wishing to be bound by any theory, it is envisioned that loss of CD59 may lead to increased inflammatory injury. A decrease in urinary CD59 was observed by the inventors in multiple studies to be associated with AKI.

Hepcidin was found by the inventors to decrease in AKI. Hepicidin mediates intracellular iron sequestration. It has been shown to be decreased in the urine of patients with AKI after cardiac surgery. It is very highly suppressed in both the early study and the rat study. Interestingly, it is expressed at low levels in both groups of the RRT study. This may indicate that the urine concentration of hepcidin is greatly decreased in AKI of any magnitude. Thus, it may prove to be a good early marker but not able to differentiate differences in the magnitude of AKI.

### I. METHODS FOR PROTEIN DETECTION

Expression of various protein markers in a sample can be analyzed by a number of methodologies, many of which are known in the art and understood by the skilled artisan including, but not limited to, immunohistochemical and/or Western analysis, FACS, protein arrays, mass spectrometry, quantitative blood based assays (e.g., serum ELISA), an enzyme-linked immunoassay, an AQUA system assay, a radioimmunoassay, an immunoprecipitation, a nephelometry assay or an immunonephelometry assay, a fluorescence immunoassay, a chemiluminescent assay, an immunoblot assay, a lateral flow assay, a flow cytometry assay, an electrochemical assay, a Luminex™ suspension array assay, a SearchLight™ protein array assay, a dipstick test, a membrane-based test strip, a point of care test, and a particulate-based assay (e.g., a particulate-based suspension array assay performed using the Bio-Plex® system; Bio-Rad Laboratories, Hercules, Calif., USA).

Liquid Chromatography-Tandem Mass Spectrometry (LC-MS/MS)

Liquid chromatography-mass spectroscopy (LC-MS/MS) may be used to detect one or more urinary proteins. In some embodiments, the method may comprise performing a Multiple Reaction Monitoring (MRM) test, a selected reaction monitoring (SRM) test, or an MRM-like or SRM-like test. MRM tests can generally involve obtaining a biological sample such as a urine sample and substantially purifying or isolating protein from the sample. The protein may then be treated with a protease, such as trypsin, to fragment the proteins in the sample. LC-MS/MS may then be performed on the sample with one or more internal standards to that correspond to a biomarker or urinary protein, e.g., that is associated with a kidney disease such as AKI. The internal standard may be a known amount of an isotopically labeled peptide (e.g., labeled with C¹³ or C¹⁵) whose sequence corresponds to a protein or peptide of interest. Thus, the internal standard may separate with a peptide that corresponds to the protein of interest during liquid chromatography; however, when the internal standard peptide is ionized during mass spectrometry, the atomic mass of the internal standard will be different, e.g., several Daltons heavier. Based on the known identity and quantity of an internal standard, one may determine the identity and quantity of a protein or peptide from a biological sample such as a urine sample.

In some embodiments, the following protocol may be used. One or more unlabeled and/or isotopically labeled proteotypic peptides may be synthesized for each biomarker protein. A proteotypic peptide for angiotensinogen may be ALQDQLVLVAAK (SEQ ID NO:19). The terminal lysine or arginine of the isotopically labeled peptide may be labeled with heavy carbon (¹³C) and nitrogen (¹⁵N) so that the labeled peptide is 8 or 10 Da heavier than the unlabeled peptide, respectively. A mixture of the labeled peptides may be made and standard concentration curves may be constructed for each peptide based on one or more product ions (MS/MS product). Urine (e.g., supernatant from a 1,000×g centrifugation) may be thawed in a 37° C. water bath if needed. Urine volume may be normalized for the creatinine concentration and may be added to a 0.2% (w/v) solution (in 100 mmolar ammonium bicarbonate) Rapigest SF surfactant to make an equal volume of each sample. Each sample may be spiked with the cocktail of isotopically labeled peptides. The urine samples may be reduced, alkylated, digested with trypsin and may be loaded onto a reversed phase solid phase extraction column. The column may be a Strata-X polymeric column. The column may be eluted with 40% acetonitrile. Ten microliters of the eluted fraction may be separated on a reverse phase column. The column may be a C18 column. The peptides may be eluted from the column. The elution gradient may be a gradient of 2 to 80% acetonitrile with 0.1% formic acid. The elution time may be 30 minutes. The peptides may be injected into a mass spectrometer. The mass spectrometer may be a triple quadrupole mass spectrometer. The mass spectrometer may be a tandem quadrupole mass spectrometer. The mass spectrometer may be an orbitrap mass spectrometer. The mass spectrometer may be an AB SCIEX 5600 triple-ToF mass spectrometer. Protein abundance may be determined by comparing the summed intensity of the appropriate product ion of the endogenous peptide to the summed intensity of the peptide containing the stable isotope. The protein abundance may be determined using specialized software. The specialized software may be the Multiquant software package (ABSciex).

Mass Spectrometry Detection

In some embodiments the biomarker protein may be measured by mass spectrometry. In one embodiment the biomarker proteins or protein fragments of the proteins may be measured by Surface Enhanced Laser Desorption/Ionization (SELDI) as has been described e.g. by Vahoutte et al., 2007 (Nephrol Dial Transplant. 2007 October; 22(10):2932-43). In one embodiment the biomarker protein or protein fragments may be measured by capillary electrophoresis mass spectrometry as has been described e.g. by Mischak and Schanstra 2011 (Proteomics Clin. Appl. 2011, 5, 9-23).

Immunodetection

In some embodiments, an immunodetection method is used to detect one or more proteins, such as urinary proteins, as described herein. In some embodiments, the immunodetection method is an ELISA, a nephelometry assay, an immunonephelometry test, Luminex™-based immunoassay, or other immunoassay. Immunodetection methods may generally involve antibodies or fragments of antibodies that specifically bind to or recognize a protein marker as described herein. Antibodies can be made by any of the methods that are well known to those of skill in the art. The following methods exemplify some of the most common antibody production methods. Antibodies may be labeled with, e.g., a radioactive element used in radioimmunoassays; enzymes; a fluorescent, phosphorescent, or chemiluminescent dyes; a latex or magnetic particles; a dye crystallite, gold, silver, or selenium colloidal particles; a metal chelate; a coenzyme; an electroactive groups; an oligonucleotide, or a stable radical. For example, in some embodiments, the Human Total Angiotensinogen Assay Kit (Immuno-Biological Laboratories Co., Ltd.), a solid phase sandwich ELISA, may be used according the manufacturer's protocol to measure urinary angiotensinogen.

Polyclonal antibodies generally are produced in animals by multiple subcutaneous (sc) or intraperitoneal (ip) injections of the antigen. As used herein the term “antigen” refers to any polypeptide that comprises a portion of or the full length protein of the protein markers described herein. However, it will be understood by one of skill in the art that in many cases antigens comprise more material that merely a single polypeptide. In certain other aspects of the invention, antibodies will be generated against specific polypeptide antigens. In some cases the full length polypeptide sequences may be used as an antigen however in certain cases fragments of a polypeptide (i.e., peptides) may used. In still further cases, antigens may be defined as comprising or as not comprising certain post translational modifications such as, phosphorylated, acetylated, methylated, glycosylated, prenylated, ubiqutinated, sumoylated or NEDDylated residues. Thus one skilled in the art would easily be able to generate an antibody that binds to any particular cell or polypeptide of interest using method well known in the art.

In the case where an antibody is to be generated that binds to a particular protein or polypeptide it may be useful to conjugate the antigen or a fragment containing the target amino acid sequence to a protein that is immunogenic in the species to be immunized, e.g. keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glytaraldehyde, succinic anhydride, SOCl₂, or R¹N═C═NR, where R and R¹ are different alkyl groups.

Animals may be immunized against the immunogenic conjugates or derivatives by, for example, combining 1 mg or 1 μg of conjugate (for rabbits or mice, respectively) with 3 volumes of Freund's complete adjuvant and injecting the solution intradermally at multiple sites. One month later the animals may be boosted with about ⅕ to 1/10 the original amount of conjugate in Freund's complete adjuvant by subcutaneous injection at multiple sites. Seven to 14 days later the animals may be bled and the serum is assayed for specific antibody titer. Animals may be boosted until the titer plateaus. Preferably, the animal is boosted with the same antigen conjugate, but conjugated to a different protein and/or through a different cross-linking reagent. Conjugates also can be made in recombinant cell culture as protein fusions. Also, aggregating agents, such as alum, or other adjuvants may be used to enhance the immune response.

The invention also provides monoclonal antibodies for detecting and measuring the expression levels of the protein markers described herein. Monoclonal antibodies may be obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies. Monoclonal antibodies include, but are not limited to, mouse monoclonal antibodies, rabbit monoclonal antibodies, human monoclonal antibodies, and chimeric antibodies.

For example, monoclonal antibodies of the invention may be made using the hybridoma method first described by Kohler & Milstein (1975), or may be made by recombinant DNA methods (U.S. Pat. No. 4,816,567).

In the hybridoma method, a mouse or other appropriate host animal (such as a rabbit) is immunized as described above to elicit lymphocytes, such as plasma cells, that produce or are capable of producing antibodies that will specifically bind to the protein used for immunization. Alternatively, lymphocytes may be immunized in vitro. Lymphocytes may then be fused with myeloma cells using a suitable fusing agent, such as polyethylene glycol, to form a hybridoma cell (Goding, 1986).

The hybridoma cells thus prepared may be seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high level expression of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Culture medium in which hybridoma cells are growing is assayed for production of monoclonal antibodies directed against the target antigen. Preferably, the binding specificity of monoclonal antibodies produced by hybridoma cells is determined by immunoprecipitation or by an in vitro binding assay, such as radioimmunoas say (RIA) or enzyme-linked immunoabsorbent assay (ELISA). The binding affinity of the monoclonal antibody can, for example, be determined by the Scatchard analysis of Munson & Pollard (1980).

After hybridoma cells are identified that produce antibodies of the desired specificity (e.g., specificity for a phosphorylated vs. un-phosphorylated antigen), affinity, and/or activity, the clones may be subcloned by limiting dilution procedures and grown by standard methods (Goding, 1986). Suitable culture media for this purpose include, for example, Dulbecco's Modified Eagle's Medium or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxyapatite chromatography, gel electrophoresis, dialysis, or affinity chromatography.

Rabbit monoclonal antibodies may also be used for measuring expression levels of the marker proteins. Methods for generating rabbit monoclonal antibodies are known in the art. (See U.S. Pat. Nos. 5,675,063 and 7,429,487, and Spieker-Polet et al., 1995).

DNA encoding monoclonal antibodies may be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (Morrison et al., 1984), or by covalently joining to the immunoglobulin coding sequence all or part of the coding sequence for a non-immunoglobulin polypeptide. In that manner, “chimeric” or “hybrid” antibodies are prepared that have the binding specificity for any particular antigen described herein.

Typically, such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for the target antigen and another antigen-combining site having specificity for a different antigen. Chimeric or hybrid antibodies also may be prepared in vitro using known methods in synthetic protein chemistry. Other methods known in the art, such as phage display and yeast display, may also be used to generate antibodies that specifically bind to the protein markers.

For some applications, the antibodies may be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; biotin (which enables detection of the antibody with an agent that binds to biotin, such as avidin; or an enzyme (either by chemical coupling or polypeptide fusion), such as alkaline phosphatase, beta-galactosidase or horseradish peroxidase.

Any method known in the art for separately conjugating the antibody to the detectable moiety may be employed, including those methods described by Hunter et al., 1962; David et al., 1974; Pain et al., 1981; and Nygren, 1982.

The antibodies may be employed in any known assay method, such as competitive binding assays, direct and indirect sandwich assays, and immunoprecipitation assays (Zola, 1987). For instance the antibodies may be used in the detection assays described herein.

Additionally, antibodies may be used in competitive binding assays. These assays rely on the ability of a labeled standard (which may be a purified target antigen or an immunologically reactive portion thereof) to compete with the test sample analyte for binding with a limited amount of antibody. The amount of antigen in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three part complex (see for example U.S. Pat. No. 4,376,110). The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

Commercially available antibodies against the protein markers may be used for measuring expression levels of protein markers. For example, The Human Total Angiotensinogen Assay Kit (Immuno-Biological Laboratories Co., Ltd.), a solid phase sandwich ELISA, may be used according the manufacturer's protocol to measure urinary angiotensinogen. In some embodiments, it is anticipated that a tissue sample may be analyzed by automated quantitative analysis (AQUA) system or immunohistochemistry (IHC).

In some embodiments the biomarker protein may be detected by a nucleic acid aptamer which binds to the biomarker protein. In some embodiments the biomarker protein may be detected by a peptoid which binds to the biomarker protein.

Particulate-Based Assays

In general, particle-based assays use a capture-binding partner, such as an antibody or an antigen in the case of an immunoassay, coated on the surface of particles, such as microbeads, crystals, chips, or nanoparticles. Particle-based assays may be effectively multi-plexed or modified to assay numerous variables of interest by incorporating fluorescently labeled particles or particles of different sizes in a single assay, each coated or conjugated to one or more labeled capture-binding partners. The use of sensitive detection and amplification technologies with particle-based assay platforms known in the art has resulted in numerous flexible and sensitive assay systems to choose from in performing a method described herein. For example, a multi-plex particle-based assay such as the suspension array Bio-Plex® assay system available from Bio-Rad Laboratories, Inc. (Hercules, Calif.) and Luminex, Inc. (Austin, Tex.) may be useful in evaluating expression of protein marker in a sample.

Reverse Phase Protein Array (RPPA)

In some embodiments, reverse phase protein array described in U.S. Publication No. 2008/0108091 is used for measuring the expression levels of the marker proteins. Tissue or cellular lysates can be obtained by mixing tissue sample material with lysis buffer and then serially diluted (e.g., ½, ¼, ⅛, 1/16, 1/32, 1/64, 1/128) with additional lysis buffer. Dilutions can be automated, for example, using a Tecan liquid handling robot or other similar device. This material can be printed/spotted onto a substrate, such as nitrocellulose-coated glass slides (FAST Slides, Schleicher & Schuell BioScience, Inc. USA, Keene, N.H.) with an automated GeneTac arrayer (Genomic Solutions, Inc., Ann Arbor, Mich.) or other similar devices. In certain embodiments, as many as 80 samples can be spotted in 8 serial dilutions on a single substrate. Serial dilutions can provide a slope and intercept allowing relative quantification of individual proteins. Typically, measurements of protein are compared to control peptides allowing absolute quantification.

Typically, after slide printing, the same stringent conditions for slide blocking, blotting and antibody incubation used for Western blotting may be applied prior to the addition of the primary antibody. The DAKO (Copenhagen, Denmark) signal amplification system can be used to detect and amplify antibody-binding intensity. Signal intensity is measured by scanning the slides and quantifying with software, such as the MicroVigene automated RPPA software (VigeneTech Inc., Massachusetts), to generate sigmoidal signal intensity-concentration curves for each sample. To accurately determine absolute protein concentrations, standard signal intensity-concentration curves for purified proteins/recombinant peptides of known concentration are generated for comparison with the samples in which protein concentrations are unknown. The RPPAs can be quantitative, sensitive, and reproducible. RPPA may also be validated with one or more stable loading controls.

Nephelometry Assay

In some embodiments, a nephelometry assay or a immunonephelometry assay may be used to detect or measure a biomarker or urinary protein. Various commercial systems are available for performing nephelometry assays, such as a Behring nephelometer system (BNA, BN II), the Aurora nephelometer and a Beckman Array Protein System Nephelometer. Various nephelometry techniques are known which may be used with the present invention including, but not limited to, those described in Nicol et al. (2011) and Finney et al. (1997) which are incorporated by reference in their entirety.

Lateral Flow Tests

Lateral flow tests may also be referred to as immunochromatographic strip (ICS) tests or simply strip-tests. In general, a lateral flow test is a form of assay in which the test sample flows laterally along a solid substrate via capillary action, or alternatively, under fluidic control. Such tests are often inexpensive, require a very small amount (e.g., one drop) of sample, and can typically be performed reproducibly with minimal training.

Exemplary lateral flow device formats include, but are not limited to, a dipstick, a card, a chip, a microslide, and a cassette, and it is widely deomonstrated in the art that the choice of format is largely dependent upon the features of a particular assay. Lateral flow devices provide many options to the ordinarily skilled artisan for detecting a protein-antibody complex in a sample using a lateral flow assay (e.g., U.S. Pat. Nos. 7,344,893, 7,371,582, 6,136,610, and U.S. Patent Applications, 2005/0250141 and 2005/0047972, each incorporated herein by reference.)

In related embodiments, an ELISA assay may be performed in a rapid flow-through, lateral flow, or strip test format. Various methods of detection may be used in a lateral flow immunoassay including, for example, the detection of a colored particle (e.g., latex, gold, magnetic particle, fluorescent particle). In certain embodiments, a lateral flow assay may comprise a sandwich ELISA assay specific for a protein marker.

Detecting or Predicting AKI

Quantified protein expression data from subjects with known treatment outcomes can be analyzed using known programs and algorithms, and mathematical equations or models for calculating risk for the relevant outcomes are generated and the thresholds (cutoff points) are defined to classify subjects into risk groups. Prediction or estimation of risk can be made by a number of methodologies, many of which are known in the art and understood by the skilled artisan including, but not limited to threshold values for individual proteins, use of ratio or combinations of proteins, artificial neural networks, multivariate linear regression, nearest related neighbor and Cox proportional hazard models.

In some embodiments, a ratio of two biomarker proteins may be used to predict acute kidney injury, worsening of acute kidney injury, death, length of hospital stay, length of intensive care unit stay, recovery from acute kidney injury, development of chronic kidney disease, worsening of chronic kidney disease or end stage renal disease. The biomarker ratio consists of the urine concentration of a biomarker protein from group (a) or group (c) divided by the concentration of a biomarker protein from group (b).

In some embodiments, a ratio of multiple biomarker proteins may be used to predict acute kidney injury, worsening of acute kidney injury, death, length of hospital stay, length of intensive care unit stay, recovery from acute kidney injury, development of chronic kidney disease, worsening of chronic kidney disease or end stage renal disease. The biomarker ratio consists of a value derived from the urine concentration of one or more biomarker proteins from group (a) or group (c) divided by a value derived from the concentration of a biomarker protein from group (b). The derived numbers may be a mean, median, geometric mean, weighted mean or other value derived by statistical methods.

An artificial neural network (ANN) may be created using the concentration of the biomarker proteins to predict acute kidney injury, worsening of acute kidney injury, death, length of hospital stay, length of intensive care unit stay, recovery from acute kidney injury, development of chronic kidney disease, worsening of chronic kidney disease or end stage renal disease. ANNs are a machine learning model consisting of input and output nodes and at least one hidden node. A regression process of repeatedly adjusting the weights of the nodes is stopped when the resulting error function is minimized. One example of the use of ANN for prediction of clinical outcomes is Mueller et al, 2006 (BMC Med Inform Decis Mak. 2006; 6: 11).

A multivariate linear regression may be used to create a prediction model to predict acute kidney injury, worsening of acute kidney injury, death, length of hospital stay, length of intensive care unit stay, recovery from acute kidney injury, development of chronic kidney disease, worsening of chronic kidney disease or end stage renal disease. Biomarker protein concentrations as well as clinical variables may be used as inputs. Biomarker concentrations may be log transformed. Stepwise regression may be used to estimate which biomarkers or other variables are the best predictors. Models may be generated using for example SAS (Cary, N.C.). An example of the use of multivariate linear regression is e.g. Neuhouser et al. 2003 (Public Health Nutr.2003 October; 6(7):703-9).

A nearest related neighbor or k-related neighbor algorithms may be used to create a prediction model to predict acute kidney injury, worsening of acute kidney injury, death, length of hospital stay, length of intensive care unit stay, recovery from acute kidney injury, development of chronic kidney disease, worsening of chronic kidney disease or end stage renal disease. Biomarker protein concentrations as well as clinical variables may be used as inputs. An example of the use of nearest related neighbor classifiers is, e.g., Oates et al. 2010 (Arthritis Rheum 2010; 62 Suppl 10:1403 D01: 10.1002/art.29169)

In one embodiment, a multivariate COXPH model is used as the prediction model. Two or three or four or more multiple-component classifiers, each in the form of a mathematical equation, may be created based on the fitting of the multivariate COXPH models to the features using the entire training set. Each component in an equation is a protein or other variable that may be weighted, for example, by the estimated logarithm of the hazard ratio derived from the COXPH modeling for outcomes. The mathematical equations calculate Risk Scores (RS) for each patient of the training set. The higher the RS, the higher the risk of outcomes. The cutoff points are defined by the lower and upper tertiles of the RS, classifying patients into, for example, three groups: the lowest risk (RS less than or equal to the lower tertile), the middle risk (RS higher than the lower tertile but less than the upper tertile), and the highest risk (RS higher than or equal to the upper tertile). The classifiers and the cutoff points may be cross-validated using patient data from an independent study. Kaplan-Meier survival analysis may be used to show that the three risk groups of the validation set are significantly different in outcomes (e.g., p<0.01 in log rank test), and/or the outcome rate of the patients in the high risk group is significantly higher than that of the patients in the low risk group (e.g., p<0.001, 0.0001, 0.00001, or 0.000001

In some embodiments, the risk score (RS) of a patient equals to the sum of products, wherein each product may be the expression level of each protein marker in the panel in the patient sample multiplied by a coefficient reflecting its relative intra-set contribution to the risk of outcomes. The coefficient of each marker and the predetermined thresholds or cutoff points for classifying the patient into, for example, a high risk, an intermediate risk and a low risk group are determined based on samples from patients with known outcomes. The coefficients and thresholds in the mathematical equation may vary if a different assay system is used, and may be established and validated using clinical samples for each assay system. For example, these parameters may be established and validated for using an immunodetection method or LC-MS/MS method to measure protein expression levels of the markers. In some embodiments, the RS is calculated using an automated program in a computer.

In some embodiments, the expression level of a protein marker used in predicting the risk of an aspect of AKI (e.g., the presence of AKI, severe AKI, a worsening AKI, etc.) is an average value, a median value, or a mean value of the expression level measured in the patient sample. In some embodiments, the expression level of a protein marker used in predicting the risk of AKI and responsiveness to a therapy is normalized using a reference level. In some embodiments, the normalized expression level of the marker protein is calculated as a ratio of or difference between the marker protein and reference expression levels, on the original or on a log scale, respectively.

The methods described herein may also be automated in whole or in part. For example, the expression levels of one or more biomarkers may be entered into a computer or other automated machines for determining a risk score based on one or more of the algorithms described herein and/or predicting likelihood, onset, duration, or outcome (e.g., survival probability) of AKI for a patient. A report summarizing the result of the determination can be generated from the computer or other automated machines. The report may include results of risk scores, classifying the patient as having, for example, high, middle, or low risk (e.g., of having AKI or severe AKI, or death resulting from AKI, or recovering from AKI, or developing chronic kidney disease or developing worsening chronic kidney disease or developing end stage renal disease).

### II. KITS

The technology herein includes kits for evaluating presence, absence, or amount of one or more urinary proteins as described herein in a sample. A “kit” refers to a combination of physical elements. For example, a kit may include, for example, one or more components such as probes, including without limitation specific primers, antibodies, a protein-capture agent, a reagent, an instruction sheet, and other elements useful to practice the technology described herein. The kits may include one or more primers, such as primers for PCR, to detect methylation of one or more of the genes as described herein. These physical elements can be arranged in any way suitable for carrying out the invention.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a single vial. The kits of the present invention also will typically include a means for containing the an antibody or other construct for detecting a urinary protein as described herein, and any other reagent containers in close confinement for commercial sale. Such containers may include injection or blow-molded plastic containers into which the desired vials are retained.

In some embodiments, the kit may be an immunodetection kit for use with the immunodetection methods described above, e.g., to detect one or more urinary protein or peptide. The kit may comprise one or more monoclonal antibodies. In certain embodiments, the first antibody that binds to the a urinary protein, polypeptide and/or peptide may be pre-bound to a solid support, such as a column matrix and/or well of a microtitre plate. The immunodetection reagents of the kit may take any one of a variety of forms, including those detectable labels that are associated with and/or linked to the given antibody. Detectable labels that are associated with and/or attached to a secondary binding ligand are also contemplated. Exemplary secondary ligands are those secondary antibodies that have binding affinity for the first antibody. Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and/or all such labels may be employed in connection with the present invention.

In some embodiments, the kit may consist of a point-of-care test which may be used at or near the site of patient care.

A kit will also include instructions for employing the kit components as well the use of any other reagent not included in the kit. Instructions may include variations that can be implemented. It is contemplated that such reagents are embodiments of kits of the invention. Such kits, however, are not limited to the particular items identified above and may include any reagent used for the manipulation or characterization of a urinary protein as described herein (e.g., angiotensinogen).

### III. BIOCHIPS

A biochip is also provided. The biochip may comprise a solid substrate comprising a attached nucleic acid sequence that is capable of hybridizing to a urinary protein as described herein. Various biochips are known in the art which may be used with the present invention. Biochip Array Technology (BAT) is an assay technology that may be used for multi-analyte screening of biological samples, such as one or more urine samples, in a rapid, accurate and easy to use format. For example, various biochip analyzers or biochip immunoassays may be used to detect one or more urinary protein of the present invention. In some embodiments, a biochip analyzer or biochip immunoassay from, e.g., Randox Laboratories may be used with the present invention. The biochip may use a protein, antibody, aptamer, peptide, peptoid, organic chemical compound, or other construct to detect a urinary protein.

The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Urinary Proteins Associated with Acute Kidney Injury (AKI)

Proteomic Analysis. Two studies were done in urine from patients who had cardiac surgery and one study was done in a rat model of AKI. The first human study (RRT study) was designed to identify candidates that predict severe renal failure requiring renal replacement therapy (RRT). In the RRT study the inventors used proteomic analysis to identify angiotensinogen as a biomarker to predict severe AKI. The inventors confirmed the ability of urinary angiotensinogen and the angiotensinogen to creatinine ratio to predict severe AKI. The second human study (EARLY study) was designed to identify candidate urine biomarkers that occur early in acute kidney injury. The third study (RAT study) was designed to identify markers that occur in a rat model of AKI. The inventors used the data from all three proteomic studies to determine the AKI biomarkers that were useful for predicting both early AKI and severe AKI. The use of human and rat AKI samples enhanced the generalizability of the candidate markers across multiple causes of AKI and between species.

RRT Study.

Urine Samples in RRT Study.

The Southern Acute Kidney Injury Network (SAKInet) was formed in 2007 to collect samples from patients who developed AKI after cardiac surgery with the goal of testing the diagnostic and prognostic accuracy of previously described AKI biomarkers and identifying novel ones. Urine samples were obtained from patients who had cardiac surgery at one of the SAKInet institutions (the Medical University of South Carolina, Duke University, George Washington University or University of Tennessee College of Medicine in Chattanooga). Prior to collection, informed consent was obtained in accordance with the Institutional Review Board approved protocol at each member institution. Samples were collected and stored using a rigorous standard operating procedure (SOP). Most patients were catheterized and urine was collected preferentially from the Foley tube or the urometer and processed immediately. Urine specimens were treated with a reversible, serine and cysteine protease inhibitor cocktail tablet (Roche, Complete mini, EDTA-free) at a concentration of 1 tablet per 50 ml of urine. The urine was centrifuged for 10 minutes at 1,000×g and the supernatant was immediately stored at −80° C. in polypropylene tubes that had been previously washed with 100% acetonitrile in order to minimize sample contamination with plastic polymer.

Patient Selection in RRT Study.

The SAKInet SOP for urine collection is primarily focused on collection of urine samples from patients who have developed AKI after cardiac surgery. The goal is to collect urine samples as early as possible after AKIN serum creatinine criteria are met (increase in serum creatinine≧0.3 mg/dL or ≧50% from baseline) (Mehta et al., 2007).

Collections are made in the surgical ICUs. Inclusion criteria are consent by the patient or appropriate surrogate, surgery of the heart or ascending aorta and development of AKI within 3 days of surgery. The only exclusion criterion is a baseline serum creatinine greater than 3 mg/dL. Urine samples were stored at −80° C. and shipped to MUSC on dry ice. Samples used in this study were selected from among the stored samples to fit the criteria described in the results section.

Proteomic Analysis in RRT Study

Trypsin Digestion. Urine (supernatant from the 1,000×g centrifugation) was thawed in a 37° C. water bath and digested in-solution with trypsin using the following protocol. One hundred μL of each sample was diluted with 100 μL of 0.2% Rapigest SF surfactant (Waters) in 100 mM ammonium bicarbonate. To account for technical variability in the digestion and LC-MS/MS protocols, 200 ng of the internal standard recombinant HIV protein gp160 (Bioclone, Inc) was spiked into each sample. Proteins were denatured by the addition of 5 mM dithiothreitol and heated to 60° C. for 30 min. After cooling to room temperature, proteins were alkylated by the addition of 12 mM iodoacetamide and incubation at room temperature in the dark for 30 minutes. Proteins were digested with 10 μg of trypsin (Applied Biosystems, TPCK treated with CaCl₂) overnight at 37° C.

LC-MS/MS.

Each digested sample was pre-fractionated using offline reversed phase solid phase extraction (SPE). The Strata-X SPE cartridge (Phenomenex; 30 mg/mL) was activated and equilibrated by application of 1 mL methanol followed by 1 mL of 0.1% formic acid in water. The sample was loaded onto the SPE column, and a series of elutions containing progressively higher concentrations of acetonitrile (10%, 15%, 20%, 25%, 30%, 35%, 40%, 50%, and 60%) in 0.1% formic acid were performed to separate the sample into fractions of increasing hydrophobicity. The 10% and 15% eluates were combined, as were the 50% and 60% eluates. Sample fractions were completely dried in a centrifugal vacuum concentrator, and each fraction was reconstituted in 50 μl of mobile phase A (98% H₂O, 0.1% formic acid; 2% acetonitrile). Fractions from each elution were analyzed by liquid chromatography tandem mass spectrometry. Five μL of each fraction was injected onto an Acclaim PepMap100 trap column (100 μm ID×2 cm, C18, 5 μm, 100 Å; Thermo Scientific), and washed with 100% mobile phase A for 10 minutes at 2 μL/minute. The fraction was then separated on an Acclaim PepMap100 analytical column (75 μm ID×15 cm, C18, 3 μm, 100 Å; Thermo Scientific). The combined 50% and 60% elution fractions were separated using a 40 minute 2-step continuous gradient of increasing Mobile Phase B (MPB). The first step increased from 10% MPB to 40% MPB at 1.5% per minute. The second step increased from 40% MPB to 60% MPB at 1% per minute. All other elution fractions were separated using a 45 minute 2-step gradient. The first step increased from 10% to 40% MPB at 1% per minute, and the second step increased from 40% to 60% at 2% per minute. Tandem mass spectrometry was performed using an AB SCIEX Triple TOF 5600 mass spectrometer. This instrument was run in information dependent acquisition mode with the following parameters: 250 ms MS accumulation time, 50 ms MS/MS accumulation time, 20 ions selected per cycle, total cycle time of 1.3 s, 4 s dynamic exclusion time after one occurrence, and rolling collision energy. The scanning windows for the TOF-MS and MS/MS were 300 to 1250 and 55 to 2000 m/z, respectively.

Protein Identification and Quantification in RRT Study.

Acquired spectra (.wiff files) were converted to the MGF format using AB SCIEX converter version 1.1 beta. MGF files from all the fractions of each sample were merged and searched against the 2011_{—}6 release of the Human UniProtKB/Swiss-Prot database with addition of the common contaminants (20241 total entries) using the Mascot search engine with trypsin as the specified enzyme. Carbamidomethyl (C) was selected as a fixed modification, and oxidation (M) and deamidation (NQ) were selected as variable modifications. Monoisotopic masses were used, and the error tolerances were 10 ppm and 0.5 Da for peptides and MS/MS fragments, respectively. Mascot search results were exported and loaded into Scaffold (Proteome Software, Inc), which used the Peptide Prophet and Protein Prophet algorithms to validate protein identifications (Keller et al., 2002; Nesvizhskii et al., 2003). The Scaffold quantitative values of identified proteins were normalized to the internal standard recombinant HIV protein present in each biological sample.

Angiotensinogen ELISA in RRT Study.

The Human Total Angiotensinogen Assay Kit (Immuno-Biological Laboratories Co., Ltd.), a solid phase sandwich ELISA, was used according the manufacturer's protocol to measure urinary angiotensinogen. Urine samples were diluted 1:8 in EIA buffer. 100 μL of diluted sample was added to the appropriate well and incubated for 60 minutes at 37° C. The plate was then washed 7 times by pipetting 250 μl of the provided wash buffer into each well using a multichannel, repeating pipet. After drying the plate, 100 μl of 30× diluted HRP-conjugated anti-angiotensinogen antibody was added to each well and incubated for 30 minutes at 37° C. The plate was washed 9 times as before and dried. 100 μL of chromogen (TMB) was added to each well, and the plate was incubated for 30 minutes in the dark at room temperature. One hundred μL of stop solution was added to each well, and the absorbance was measured at 450 nm using a SpectraMAX 340PC 96-well plate reader. The linear range of the assay is 20 to 0.31 ng/mL. Intra- and inter-assay variability (coefficient of variation) were calculated by measuring the standards and three selected biological samples in quadruplicate once, and in duplicate on all remaining plates. Values for intra- and interassay variability were 2.4% and 9.9%, respectively. Data were analyzed using Softmax Pro3.1.2. Samples whose values were above the upper limit of quantification for the assay were diluted 1:10 in EIA buffer and re-run on a separate plate.

Urine Creatinine Determination.

Urine creatinine was used to correct the urine angiotensinogen concentration and values were reported as the ratio of angiotensinogen in ng/ml to creatinine in mg/ml (uAnCR, ng/mg). Urine creatinine was measured using the Jaffe assay. 3 μL of sample was combined with 100 μL of 1% picric acid (Sigma-Aldrich), 100 μL of 0.75 M NaOH (Genomic Solutions), and 300 μL distilled deionized H₂O, Samples were incubated at room temperature for 15 minutes and absorbance at 490 nm was measured using a SpectraMAX 340PC 96-well plate reader. Data were analyzed using Softmax Pro 3.1.2.

Statistical Analysis in RRT Study.

Differentially abundant proteins identified and quantified by LC-MS/MS were selected using the Wilcoxon Rank-Sum test with a significance threshold of p<0.05. This test was used because it has been previously shown to be a robust test for the identification of candidate biomarkers in proteomics studies with small sample sizes (Dakna et al., 2010). In verification studies, the Kruskal-Wallis ANOVA on Ranks test and the post hoc Dunn's test for pairwise comparison (SigmaPlot) were used to evaluate differential abundance of uAnCR in multiple groups. Receiver operator characteristic curves (SigmaPlot) were constructed to determine the predictive power of uAnCR. The area under the ROC curve (AUC) was used as an estimate of an overall accuracy of the biomarker. An AUC of 1.0 represents 100% accuracy, whereas an AUC of 0.5 indicates 50% accuracy, which is no better than random chance. ROC curves were considered statistically significant if the AUC differed from 0.5, as determined by the z-test. Optimal cut-offs were determined by selecting the data point that minimized the geometric distance from 100% sensitivity and 100% specificity on the ROC curve (Pepe, 2004). Additionally, cut-offs that maximized the positive likelihood ratio and minimized the negative likelihood ratio were reported since they could be useful in assigning high or low risk to a patient Likelihood ratios of positive and negative predictive value were used since they are insensitive to changes in prevalence (unlike PPV and NPV) and can be used to infer post-test probability. Kaplan-Meier curves were used to visualize the relationship between uAnCR and length of stay. Patients who died were censored. The log-rank test was used to compare the curves, and the Holm-Sidak test was used for post-hoc pairwise comparison.

RESULTS in RRT Study

Discovery of Candidate Prognostic AKI Biomarkers

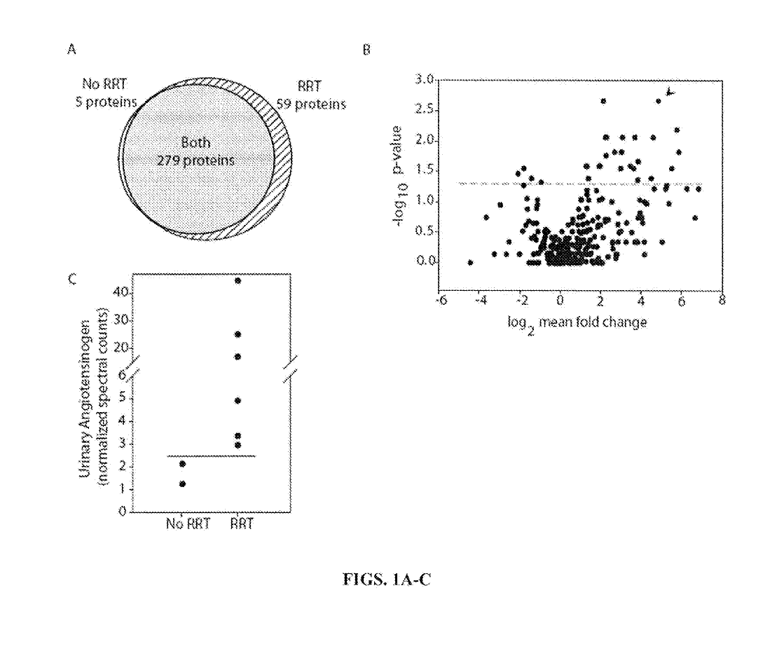

The objective was to discover candidate prognostic biomarkers of AKI using quantitative proteomic analysis of the urine. The inventors used liquid chromatography-tandem mass spectrometry to compare the urinary proteomic profiles of twelve patients who developed AKI after cardiac surgery, six of whom required renal replacement therapy (RRT) and six of whom did not. Patients were selected such that there were no differences between the two groups with respect to the distributions of gender, race, age, weight, use of intraoperative cardiopulmonary bypass, bypass time, pre-operative sCr, sample collection time, and type of surgery (see Table 1 for patient characteristics). A total of 343 proteins were identified (minimum 80% peptide identification confidence; minimum 99% protein identification confidence with at least two peptides identified per protein; calculated protein false discovery rate of 1.5%), of which 59 were unique to patients who required RRT, and five were unique to patients who did not (FIG. 1A). However, only ten of these severe AKI-specific proteins were observed in four or more of the six samples in the RRT group. Of note, one of these was the known AKI biomarker NGAL, which was only observed in patients who required RRT but did not reach statistical significance (four of six patients had detectable NGAL). To identify candidate biomarkers, relative protein abundances were estimated with correction for the internal standard. The change in the abundance of each identified protein was also described by calculating the mean fold-change between the two groups. Mean fold-change was plotted against p-value in a “volcano plot” in order to enhance selection of candidate markers that differentiated between the groups (FIG. 1B). The relative abundance of 30 proteins was statistically different from the 343 total proteins the inventors observed (see Table 2 and Table 1A). Twenty-six were elevated in the urine of patients who required RRT and four were reduced. The inventors selected angiotensinogen as the most promising candidate marker based on the combination of p-value and fold-change difference between groups (FIG. 1B). Relative abundances of angiotensinogen for the individual subjects seen in FIG. 1C show that urinary angiotensinogen discriminates with 100% accuracy between patients who required RRT and those who did not. Based on these data, the inventors attempted to verify the potential of urinary angiotensinogen as a biomarker of severe AKI after cardiac surgery.

TABLE 1
Characteristics of patients used in a discovery phase proteomics study to
identify candidate biomarkers of severe AKI

| | Biomarkers of Severe AKI | | |
|---|---|---|---|
| | No RRT | RRT | p-value |
| n | 6 | 6 | |
| Male | 4 (67%) | 4 (67%) | 1 |
| Caucasian | 6 (100%) | 6 (100%) | 1 |
| Age (yrs) | 63.83 | 7250 | 0.29 |
| Weight (kg) | 75.92 | 85.32 | 0.33 |
| Bypass | 4 (67%) | 4 (67%) | 1 |
| Bypass Time (hrs) | 1:49 | 1:50 | 1 |
| Pre-op sCr (mg/dl) | 1.30 | 1.37 | 0.76 |
| sCr at Collection (mg/dl) | 1.88 | 2.60 | 0.37 |
| Collection Time (post-op hrs) | 29.20 | 38.00 | 0.3 |
| Max sCr (mg/dl) | 2.10 | 4.23 | |
| Day of Max sCr (post-op) | 1.73 | 4.83 | 0.006 |
| RRT 10 days | 0 (0%) | 6 (100%) | 0.002 |
| Death | 0 (0%) | 4 (67%) | 0.06 |

Groups were compared using the Fisher Exact test for count data, and the t-test or Mann-Whitney U test for continuous variables.

TABLE 2
Candidate Prognostic AKI Biomarkers identified by LC-MS/MS

| | Uniprot | | | | |
|---|---|---|---|---|---|
| | Accession | | | Mean Fold | |
| Identified Proteins (349) | Number | Mean No RRT | Mean RRT | Change | p-value |
| Angiotensinogen | P01019 | 1.69 | 16.33 | 9.673988599 | 0.002 |
| Serum albumin | P02768 | 653.89 | 2892.67 | 4.423774872 | 0.002 |
| Apolipoprotein A-IV | P06727 | 2.36 | 21.45 | 9.086038523 | 0.006 |
| Complement C3 | P01024 | 8.82 | 50.09 | 5.680328859 | 0.009 |
| Vitamin D-binding protein | P02774 | 4.51 | 54.58 | 12.10943811 | 0.009 |
| Complement C4-B | P0C0L5 | 2.71 | 23.25 | 8.583050831 | 0.009 |
| Superoxide diamutase [Cu—Zn] | P00441 | 10.10 | 23.91 | 2.366717399 | 0.009 |
| Epididymai secretory protein E1 | P61916 | 3.17 | 10.39 | 3.278868509 | 0.009 |
| Phosphatidylethanolamine-binding pritein 1 | P30086 | N/A | 5.90 | N/A | 0.02 |
| Complement factor D | P00746 | N/A | 5.81 | N/A | 0.02 |
| Coactosin-like protein | Q14019 | N/A | 3.70 | N/A | 0.02 |
| Serotransferrin | P02787 | 72.61 | 472.14 | 6.502131439 | 0.02 |
| profilin-1 | P07737 | 5.87 | 24.04 | 4.097798143 | 0.02 |
| Cystatin-8 | P04080 | 0.43 | 4.94 | 11.52838915 | 0.02 |
| Fibrinegen alpha chain | P02671 | 7.33 | 35.39 | 4.828841123 | 0.02 |
| Brain acid soluble protein 1 | P80723 | 0.87 | 5.05 | 5.82321387 | 0.02 |
| Zinc-alpha-2-glycoprotein | P25311 | 112.02 | 228.64 | 2.040958185 | 0.03 |
| Alpha-1-antitrypsin | P01009 | 21.55 | 239.32 | 11.1065304 | 0.03 |
| Alpha-1-acid glycoprotein 1 | P02763 | 44.94 | 112.82 | 2.510373307 | 0.03 |
| Hemopexin | P02790 | 7.94 | 30.11 | 3.790161845 | 0.03 |
| Fibrinogen beta chain | P02675 | 3.65 | 14.29 | 3.920601471 | 0.03 |
| Pigment epithelium-derived factor | P36955 | 2.50 | 22.71 | 9.083831432 | 0.03 |
| Fatty acid-binding protein, adipocyte | P15090 | 6.25 | 9.99 | 1.598037597 | 0.03 |
| Alpha-1-acid glycoprotein 2 | P19652 | 18.15 | 47.56 | 2.6201484 | 0.04 |
| Metallothionein-2 | P02795 (+2) | 1.43 | 19.25 | 13.47439598 | 0.04 |
| Apolipoprotein A-1 | P02647 | 2.39 | 20.36 | 8.528718575 | 0.04 |
| Keratin, type II cytoskeletal 5 | P13647 | 5.74 | 2.16 | −1.736359877 | 0.03 |
| Secreted Ly-6/uPAR-related protein 1 | P55000 | 4.49 | 3.18 | −1.413606825 | 0.03 |
| Non-secretory ribonuclease | P10153 | 17.15 | 7.72 | −2.221003585 | 0.04 |
| Keratin, type II cytoskeletal 1 | P04264 | 35.85 | 18.85 | −1.901670542 | 0.05 |

Protein abundance is reported as normalized spectral counts

TABLE 1A

| | | | Wilcoxon | | |
|---|---|---|---|---|---|
| | Uniprot | | Rank- | | |
| | Accession | | Sum | Up or | Mean Fold |
| Identified Proteins (349) | Number | Patient 1 | p-val | Down | Change |
| Angiotensinogen OS = Homo | P01019 | 0.0000 | 0.0022 | Up | 29.02197 |
| sapiens GN = AGT PE = 1 | | | | | |
| SV = 1 | | | | | |
| Serum albumin OS = Homo | P02768 | 577.3388 | 0.0022 | Up | 4.423775 |
| sapiens GN = ALB PE = 1 | | | | | |
| SV = 2 | | | | | |
| Apolipoprotein A-IV | P06727 | 0.0000 | 0.0065 | Up | 54.51623 |
| OS = Homo sapiens | | | | | |
| GN = APOA4 PE = 1 SV = 3 | | | | | |
| Complement C3 OS = Homo | P01024 | 0.0000 | 0.0087 | Up | 8.520493 |
| sapiens GN = C3 PE = 1 SV = 2 | | | | | |
| Vitamin D-binding protein | P02774 | 1.7710 | 0.0087 | Up | 24.21888 |
| OS = Homo sapiens GN = GC | | | | | |
| PE = 1 SV = 1 | | | | | |
| Complement C4-B | P0C0L5 | 0.0000 | 0.0087 | Up | 12.87458 |
| OS = Homo sapiens | | | | | |
| GN = C4B PE = 1 SV = 1 | | | | | |
| Superoxide dismutase [Cu—Zn] | P00441 | 0.0000 | 0.0087 | Up | 4.733435 |
| OS = Homo sapiens | | | | | |
| GN = SOD1 PE = 1 SV = 2 | | | | | |
| Epididymal secretory | P61916 | 1.7710 | 0.0087 | Up | 4.918303 |
| protein E1 OS = Homo | | | | | |
| sapiens GN = NPC2 PE = 1 | | | | | |
| SV = 1 | | | | | |
| Phosphatidylethanolamine- | P30086 | 0.0000 | 0.0152 | Up | N/A |
| binding protein 1 OS = Homo | | | | | |
| sapiens GN = PEBP1 PE = 1 | | | | | |
| SV = 3 | | | | | |
| Complement factor D | P00746 | 0.0000 | 0.0152 | Up | N/A |
| OS = Homo sapiens | | | | | |
| GN = CFD PE = 1 SV = 5 | | | | | |
| Coactosin-like protein | Q14019 | 0.0000 | 0.0152 | Up | N/A |
| OS = Homo sapiens | | | | | |
| GN = COTL1 PE = 1 SV = 3 | | | | | |
| Serotransferrin OS = Homo | P02787 | 20.3662 | 0.0152 | Up | 6.502181 |
| sapiens GN = TF PE = 1 SV = 3 | | | | | |
| Profilin-1 OS = Homo | P07737 | 0.0000 | 0.0152 | Up | 8.195592 |
| sapiens GN = PFN1 PE = 1 | | | | | |
| SV = 2 | | | | | |
| Cystatin-B OS = Homo | P04080 | 0.0000 | 0.0152 | Up | 58.14195 |
| sapiens GN = CSTB PE = 1 | | | | | |
| SV = 2 | | | | | |
| Fibrinogen alpha chain | P02671 | 4.4274 | 0.0173 | Up | 4.828841 |
| OS = Homo sapiens | | | | | |
| GN = FGA PE = 1 SV = 2 | | | | | |
| Brain acid soluble protein 1 | P80723 | 0.8855 | 0.0216 | Up | 14.55805 |
| OS = Homo sapiens | | | | | |
| GN = BASP1 PE = 1 SV = 2 | | | | | |
| Zinc-alpha-2-glycoprotein | P25311 | 54.9003 | 0.0260 | Up | 2.44915 |
| OS = Homo sapiens | | | | | |
| GN = AZGP1 PE = 1 SV = 2 | | | | | |
| Alpha-1-antitrypsin | P01009 | 1.7710 | 0.0260 | Up | 11.10653 |
| OS = Homo sapiens | | | | | |
| GN = SERPINA1 PE = 1 | | | | | |
| SV = 3 | | | | | |
| Alpha-1-acid glycoprotein 1 | P02763 | 30.1066 | 0.0260 | Up | 2.510373 |
| OS = Homo sapiens | | | | | |
| GN = ORM1 PE = 1 SV = 1 | | | | | |
| Hemopexin OS = Homo | P02790 | 1.7710 | 0.0260 | Up | 3.790162 |
| sapiens GN = HPX PE = 1 | | | | | |
| SV = 2 | | | | | |
| Fibrinogen beta chain | P02675 | 1.7710 | 0.0260 | Up | 3.920601 |
| OS = Homo sapiens | | | | | |
| GN = FGB PE = 1 SV = 2 | | | | | |
| Pigment epithelium-derived | P36955 | 0.0000 | 0.0281 | Up | 45.41916 |
| factor OS = Homo sapiens | | | | | |
| GN = SERPINF1 PE = 1 | | | | | |
| SV = 4 | | | | | |
| Fatty acid-binding protein, | P15090 | 0.0000 | 0.0281 | Up | 7.990438 |
| adipocyte OS = Homo | | | | | |
| sapiens GN = FABP4 PE = 1 | | | | | |
| SV = 3 | | | | | |
| Alpha-1-acid glycoprotein 2 | P19652 | 3.5420 | 0.0411 | Up | 2.620148 |
| OS = Homo sapiens | | | | | |
| GN = ORM2 PE = 1 SV = 2 | | | | | |
| Metallothionein-2 | P02795 (+2) | 0.8855 | 0.0411 | Up | 22.45733 |
| OS = Homo sapiens | | | | | |
| GN = MT2A PE = 1 SV = 1 | | | | | |
| Apolipoprotein A-I | P02647 | 0.8855 | 0.0433 | Up | 14.21453 |
| OS = Homo sapiens | | | | | |
| GN = AP0A1 PE = 1 SV = 1 | | | | | |
| Thymosin beta-4-like | A8MW06 (+1) | 0.0000 | 0.0541 | Up | 38.15575 |
| protein 3 OS = Homo sapiens | | | | | |
| GN = TMSL3 PE = 2 SV = 1 | | | | | |
| Apolipoprotein C-III | P02656 | 0.0000 | 0.0606 | Up | N/A |
| OS = Homo sapiens | | | | | |
| GN = APOC3 PE = 1 SV = 1 | | | | | |
| Dermcidin OS = Homo | P81605 | 0.0000 | 0.0606 | Up | N/A |
| sapiens GN = DCD PE = 1 | | | | | |
| SV = 2 | | | | | |
| Neutrophil gelatinase- | P80188 | 0.0000 | 0.0606 | Up | N/A |
| associated lipocalin | | | | | |
| OS = Homo sapiens | | | | | |
| GN = LCN2 PE = 1 SV = 2 | | | | | |
| Insulin-like growth factor- | P08833 | 0.0000 | 0.0606 | Up | N/A |
| binding protein 1 OS = Homo | | | | | |
| sapiens GN = IGFBP1 PE = 1 | | | | | |
| SV = 1 | | | | | |
| Carbonic anhydrase 3 | P07451 | 0.0000 | 0.0606 | Up | N/A |
| OS = Homo sapiens | | | | | |
| GN = CA3 PE = 1 SV = 3 | | | | | |
| Ig heavy chain V-III region | P01767 | 0.0000 | 0.0606 | Up | N/A |
| BUT OS = Homo sapiens | | | | | |
| PE = 1 SV = 1 | | | | | |
| Heme-binding protein 2 | Q9Y5Z4 | 0.0000 | 0.0606 | Up | N/A |
| OS = Homo sapiens | | | | | |
| GN = HEBP2 PE = 1 SV = 1 | | | | | |
| Myoglobin OS = Homo | P02144 | 0.0000 | 0.0606 | Up | 114.3198 |
| sapiens GN = MB PE = 1 | | | | | |
| SV = 2 | | | | | |
| Ig heavy chain V-III region | P01766 | 0.0000 | 0.0606 | Up | 76.36752 |
| BRO OS = Homo sapiens | | | | | |
| PE = 1 SV = 1 | | | | | |
| Ribosome-binding protein 1 | Q9P2E9 | 0.0000 | 0.0606 | Up | 37.18535 |
| OS = Homo sapiens | | | | | |
| GN = RRBP1 PE = 1 SV = 4 | | | | | |
| Ig lambda chain V-III region | P01714 | 0.0000 | 0.0606 | Up | 25.04209 |
| SH OS = Homo sapiens PE = 1 | | | | | |
| SV = 1 | | | | | |
| Retinol-binding protein 4 | P02753 | 0.8855 | 0.0649 | Up | 3.468753 |
| OS = Homo sapiens | | | | | |
| GN = RBP4 PE = 1 SV = 3 | | | | | |
| Monocyte differentiation | P08571 | 1.7710 | 0.0649 | Up | 2.511817 |
| antigen CD14 OS = Homo | | | | | |
| sapiens GN = CD14 PE = 1 | | | | | |
| SV = 2 | | | | | |
| Ig mu chain C region | P01871 | 0.0000 | 0.0758 | Up | 2.515558 |
| OS = Homo sapiens | | | | | |
| GN = IGHM PE = 1 SV = 3 | | | | | |
| Fatty acid-binding protein, | P05413 | 0.0000 | 0.0887 | Up | 4.241953 |
| heart OS = Homo sapiens | | | | | |
| GN = FABP3 PE = 1 SV = 4 | | | | | |
| Complement factor B | P00751 | 0.0000 | 0.0909 | Up | 2.068697 |
| OS = Homo sapiens | | | | | |
| GN = CFB PE = 1 SV = 2 | | | | | |
| Alpha-1B-glycoprotein | P04217 | 0.8855 | 0.0931 | Up | 3.820367 |
| OS = Homo sapiens | | | | | |
| GN = A1BG PE = 1 SV = 4 | | | | | |
| Ceruloplasmin OS = Homo | P00450 | 0.8855 | 0.0931 | Up | 15.24963 |
| sapiens GN = CP PE = 1 SV = 1 | | | | | |
| Clusterin OS = Homo sapiens | P10909 | 1.7710 | 0.0931 | Up | 2.64484 |
| GN = CLU PE = 1 SV = 1 | | | | | |
| Beta-2-glycoprotein 1 | P02749 | 2.6565 | 0.0931 | Up | 2.049718 |
| OS = Homo sapiens | | | | | |
| GN = APOH PE = 1 SV = 3 | | | | | |
| Glutathione peroxidase 3 | P22352 | 0.0000 | 0.0996 | Up | 17.96893 |
| OS = Homo sapiens | | | | | |
| GN = GPX3 PE = 1 SV = 2 | | | | | |
| Plasma protease C1 | P05155 | 0.0000 | 0.0996 | Up | 5.90384 |
| inhibitor OS = Homo sapiens | | | | | |
| GN = SERPING1 PE = 1 | | | | | |
| SV = 2 | | | | | |
| Fatty acid-binding protein, | P07148 | 0.0000 | 0.1061 | Up | 41.63238 |
| liver OS = Homo sapiens | | | | | |
| GN = FABP1 PE = 1 SV = 1 | | | | | |
| SH3 domain-binding | O75368 | 0.0000 | 0.1061 | Up | 19.47238 |
| glutamic acid-rich-like | | | | | |
| protein OS = Homo sapiens | | | | | |
| GN = SH3BGRL PE = 1 SV = 1 | | | | | |
| Apolipoprotein A-II | P02652 | 0.0000 | 0.1061 | Up | 18.92865 |
| OS = Homo sapiens | | | | | |
| GN = APOA2 PE = 1 SV = 1 | | | | | |
| Ganglioside GM2 activator | P17900 | 4.4274 | 0.1277 | Up | 2.512376 |
| OS = Homo sapiens | | | | | |
| GN = GM2A PE = 1 SV = 4 | | | | | |
| Alpha-2-HS-glycoprotein | P02765 | 0.8855 | 0.1320 | Up | 1.891023 |
| OS = Homo sapiens | | | | | |
| GN = AHSG PE = 1 SV = 1 | | | | | |
| Afamin OS = Homo sapiens | P43652 | 0.0000 | 0.1515 | Up | 15.37942 |
| GN = AFM PE = 1 SV = 1 | | | | | |
| Serine protease inhibitor | Q9NQ38 | 0.0000 | 0.1515 | Up | 7.462258 |
| Kazal-type 5 OS = Homo | | | | | |
| sapiens GN = SPINK5 PE = 1 | | | | | |
| SV = 2 | | | | | |
| Carbonic anhydrase 1 | P00915 | 48.7019 | 0.1688 | Up | 2.210792 |
| OS = Homo sapiens | | | | | |
| GN = CA1 PE = 1 SV = 2 | | | | | |
| Ig lambda chain V-III region | P80748 | 0.0000 | 0.1688 | Up | 2.182007 |
| LOI OS = Homo sapiens | | | | | |
| PE = 1 SV = 1 | | | | | |
| Peptidyl-prolyl cis-trans | P62937 | 0.0000 | 0.1775 | Up | 2.057019 |
| isomerase A OS = Homo | | | | | |
| sapiens GN = PPIA PE = 1 | | | | | |
| SV = 2 | | | | | |
| Ig kappa chain V-I region | P01594 | 10.6259 | 0.1775 | Up | 1.908106 |
| AU OS = Homo sapiens | | | | | |
| PE = 1 SV = 1 | | | | | |
| Ig gamma-2 chain C region | P01859 | 5.3129 | 0.1797 | Up | 3.661886 |
| OS = Homo sapiens | | | | | |
| GN = IGHG2 PE = 1 SV = 2 | | | | | |
| N(G),N(G)- | O95865 | 0.0000 | 0.1818 | Up | N/A |
| dimethylarginine | | | | | |
| dimethylaminohydrolase 2 | | | | | |
| OS = Homo sapiens | | | | | |
| GN = DDAH2 PE = 1 SV = 1 | | | | | |
| Triosephosphate isomerase | P60174 | 0.0000 | 0.1818 | Up | N/A |
| OS = Homo sapiens | | | | | |
| GN = TPI1 PE = 1 SV = 2 | | | | | |
| Fatty acid-binding protein, | Q01469 | 0.0000 | 0.1818 | Up | N/A |
| epidermal OS = Homo | | | | | |
| sapiens GN = FABP5 PE = 1 | | | | | |
| SV = 3 | | | | | |
| Phosphatidylethanolamine- | Q96S96 | 0.0000 | 0.1818 | Up | N/A |
| binding protein 4 OS = Homo | | | | | |
| sapiens GN = PEBP4 PE = 1 | | | | | |
| SV = 3 | | | | | |
| DNA repair protein REV1 | Q9UBZ9 | 0.0000 | 0.1818 | Up | N/A |
| OS = Homo sapiens | | | | | |
| GN = REV1 PE = 1 SV = 1 | | | | | |
| Metalloproteinase inhibitor | P01033 | 0.0000 | 0.1818 | Up | N/A |
| 1 OS = Homo sapiens | | | | | |
| GN = TIMP1 PE = 1 SV = 1 | | | | | |
| Complement component C8 | P07360 | 0.0000 | 0.1818 | Up | 101.8623 |
| gamma chain OS = Homo | | | | | |
| sapiens GN = C8G PE = 1 | | | | | |
| SV = 3 | | | | | |
| Transthyretin OS = Homo | P02766 | 0.0000 | 0.1818 | Up | 14.51914 |
| sapiens GN = TTR PE = 1 | | | | | |
| SV = 1 | | | | | |
| Vesicular integral- | Q12907 | 0.8855 | 0.1926 | Up | 1.550449 |
| membrane protein VIP36 | | | | | |
| OS = Homo sapiens | | | | | |
| GN = LMAN2 PE = 1 SV = 1 | | | | | |
| Alpha-1-antichymotrypsin | P01011 | 4.4274 | 0.1970 | Up | 4.661815 |
| OS = Homo sapiens | | | | | |
| GN = SERPINA3 PE = 1 | | | | | |
| SV = 2 | | | | | |
| N-acetylmuramoyl-L- | Q96PD5 | 0.0000 | 0.1970 | Up | 4.875337 |
| alanine amidase OS = Homo | | | | | |
| sapiens GN = PGLYRP2 | | | | | |
| PE = 1 SV = 1 | | | | | |
| Abhydrolase domain- | Q961U4 | 0.0000 | 0.1970 | Up | 5.213184 |
| containing protein 14B | | | | | |
| OS = Homo sapiens | | | | | |
| GN = ABHD14B PE = 1 | | | | | |
| SV = 1 | | | | | |
| Neutrophil defensin 1 | P59665 (+1) | 0.0000 | 0.2035 | Up | 3.046032 |
| OS = Homo sapiens | | | | | |
| GN = DEFA1 PE = 1 SV = 1 | | | | | |
| Uteroglobin OS = Homo | P11684 | 0.0000 | 0.2100 | Up | 4.71133 |
| sapiens GN = SCGB1A1 | | | | | |
| PE = 1 SV = 1 | | | | | |
| Trypsin-2 OS = Homo | P07478 | 0.0000 | 0.2208 | Up | 2.554693 |
| sapiens GN = PRSS2 PE = 1 | | | | | |
| SV = 1 | | | | | |
| Lysozyme C OS = Homo | P61626 | 0.8855 | 0.2208 | Up | 16.82177 |
| sapiens GN = LYZ PE = 1 | | | | | |
| SV = 1 | | | | | |
| Prothymosin alpha | P06454 | 0.0000 | 0.2208 | Up | 9.757033 |
| OS = Homo sapiens | | | | | |
| GN = PTMA PE = 1 SV = 2 | | | | | |
| Cathepsin Z OS = Homo | Q9UBR2 | 0.0000 | 0.2208 | Up | 1.523644 |
| sapiens GN = CTSZ PE = 1 | | | | | |
| SV = 1 | | | | | |
| Pancreatic secretory trypsin | P00995 | 0.0000 | 0.2338 | Up | 7.233821 |
| inhibitor OS = Homo sapiens | | | | | |
| GN = SPINK1 PE = 1 SV = 2 | | | | | |
| Ig kappa chain V-I region | P01609 | 13.2823 | 0.2338 | Up | 2.196795 |
| Scw OS = Homo sapiens | | | | | |
| PE = 1 SV = 1 | | | | | |
| Ig kappa chain C region | P01834 | 294.8678 | 0.2403 | Up | 1.325457 |
| OS = Homo sapiens | | | | | |
| GN = IGKC PE = 1 SV = 1 | | | | | |
| Ig alpha-1 chain C region | P01876 | 1.7710 | 0.2403 | Up | 1.839486 |
| OS = Homo sapiens | | | | | |
| GN = IGHA1 PE = 1 SV = 2 | | | | | |
| Ig gamma-3 chain C region | P01860 | 13.2823 | 0.2403 | Up | 3.683046 |
| OS = Homo sapiens | | | | | |
| GN = IGHG3 PE = 1 SV = 2 | | | | | |
| Cystatin-C OS = Homo | P01034 | 0.0000 | 0.2403 | Up | 4.003539 |
| sapiens GN = CST3 PE = 1 | | | | | |
| SV = 1 | | | | | |
| Fibrinogen gamma chain | P02679 | 0.8855 | 0.2597 | Up | 3.637735 |
| OS = Homo sapiens | | | | | |
| GN = FGG PE = 1 SV = 3 | | | | | |
| N-acetylglucosamine-6- | P15586 | 1.7710 | 0.2857 | Up | 2.782568 |
| sulfatase OS = Homo sapiens | | | | | |
| GN = GNS PE = 1 SV = 3 | | | | | |
| Ubiquitin carboxyl-terminal | Q9P275 | 0.8855 | 0.2900 | Up | 2.549976 |
| hydrolase 36 OS = Homo | | | | | |
| sapiens GN = USP36 PE = 1 | | | | | |
| SV = 3 | | | | | |
| Apolipoprotein C-II | P02655 | 0.0000 | 0.3030 | Up | 8.034413 |
| OS = Homo sapiens | | | | | |
| GN = APOC2 PE = 1 SV = 1 | | | | | |
| Polyubiquitin-B OS = Homo | P0CG47 (+3) | 2.6565 | 0.3074 | Up | 2.215618 |
| sapiens GN = UBB PE = 1 | | | | | |
| SV = 1 | | | | | |
| Protein FAM3C OS = Homo | Q92520 | 0.0000 | 0.3074 | Up | 1.912173 |
| sapiens GN = FAM3C PE = 1 | | | | | |
| SV = 1 | | | | | |
| Ig gamma-1 chain C region | P01857 | 25.6792 | 0.3095 | Up | 2.453886 |
| OS = Homo sapiens | | | | | |
| GN = IGHG1 PE = 1 SV = 1 | | | | | |
| Ig lambda-2 chain C regions | P0CG05 | 35.4196 | 0.3095 | Up | 1.51741 |
| OS = Homo sapiens | | | | | |
| GN = IGLC2 PE = 1 SV = 1 | | | | | |
| SH3 domain-binding | Q9H299 | 7.9694 | 0.3095 | Up | 1.063169 |
| glutamic acid-rich-like | | | | | |
| protein 3 OS = Homo sapiens | | | | | |
| GN = SH3BGRL3 PE = 1 | | | | | |
| SV = 1 | | | | | |
| Ig heavy chain V-III region | P01781 | 0.0000 | 0.3182 | Up | 11.55887 |
| GAL OS = Homo sapiens | | | | | |
| PE = 1 SV = 1 | | | | | |
| Chromogranin-A OS = Homo | P10645 | 0.8855 | 0.3182 | Up | 7.558084 |
| sapiens GN = CHGA PE = 1 | | | | | |
| SV = 7 | | | | | |
| Ig kappa chain V-I region | P01611 | 0.0000 | 0.3268 | Up | 2.047986 |
| Wes OS = Homo sapiens | | | | | |
| PE = 1 SV = 1 | | | | | |
| Antithrombin-III OS = Homo | P01008 | 0.0000 | 0.3723 | Up | 3.244677 |
| sapiens GN = SERPINC1 | | | | | |
| PE = 1 SV = 1 | | | | | |
| Alpha-2-macroglobulin | P01023 | 0.0000 | 0.3874 | Up | 4.361402 |
| OS = Homo sapiens | | | | | |
| GN = A2M PE = 1 SV = 3 | | | | | |
| Protein AMBP OS = Homo | P02760 | 177.9833 | 0.3939 | Up | 1.223814 |
| sapiens GN = AMBP PE = 1 | | | | | |
| SV = 1 | | | | | |
| Leucine-rich alpha-2- | P02750 | 15.9388 | 0.3939 | Up | 1.779507 |
| glycoprotein OS = Homo | | | | | |
| sapiens GN = LRG1 PE = 1 | | | | | |
| SV = 2 | | | | | |
| Immunoglobulin lambda- | B9A064 | 24.7937 | 0.3939 | Up | 1.363527 |
| like polypeptide 5 | | | | | |
| OS = Homo sapiens | | | | | |
| GN = IGLL5 PE = 2 SV = 2 | | | | | |
| Ig kappa chain V-III region | P04433 | 2.6565 | 0.4156 | Up | 1.186655 |
| VG (Fragment) OS = Homo | | | | | |
| sapiens PE = 1 SV = 1 | | | | | |
| Beta-2-microglobulin | P61769 | 16.8243 | 0.4177 | Up | 1.93025 |
| OS = Homo sapiens | | | | | |
| GN = B2M PE = 1 SV = 1 | | | | | |
| 3-mercaptopyruvate | P25325 | 0.0000 | 0.4242 | Up | 2.228632 |
| sulfurtransferase OS = Homo | | | | | |
| sapiens GN = MPST PE = 1 | | | | | |
| SV = 3 | | | | | |
| Myc box-dependent- | O00499 | 0.0000 | 0.4242 | Up | 2.784615 |
| interacting protein 1 | | | | | |
| OS = Homo sapiens | | | | | |
| GN = BIN1 PE = 1 SV = 1 | | | | | |
| Glutaredoxin-1 OS = Homo | P35754 | 0.0000 | 0.4372 | Up | 2.005711 |
| sapiens GN = GLRX PE = 1 | | | | | |
| SV = 2 | | | | | |
| Gastrotropin OS = Homo | P51161 | 0.0000 | 0.4545 | Up | #DIV/0! |
| sapiens GN = FABP6 PE = 1 | | | | | |
| SV = 2 | | | | | |
| Fructose-bisphosphate | P05062 | 0.0000 | 0.4545 | Up | 9.538889 |
| aldolase B OS = Homo | | | | | |
| sapiens GN = ALDOB PE = 1 | | | | | |
| SV = 2 | | | | | |
| Thyroxine-binding globulin | P05543 | 0.0000 | 0.4545 | Up | 32.77778 |
| OS = Homo sapiens | | | | | |
| GN = SERPINA7 PE = 1 | | | | | |
| SV = 2 | | | | | |
| Complement component C9 | P02748 | 0.0000 | 0.4545 | Up | N/A |
| OS = Homo sapiens GN = C9 | | | | | |
| PE = 1 SV = 2 | | | | | |
| Phosphoglycerate kinase 1 | P00558 | 0.0000 | 0.4545 | Up | 6.05303 |
| OS = Homo sapiens | | | | | |
| GN = PGK1 PE = 1 SV = 3 | | | | | |
| Thioredoxin OS = Homo | P10599 | 0.0000 | 0.4545 | Up | N/A |
| sapiens GN = TXN PE = 1 | | | | | |
| SV = 3 | | | | | |
| Alpha-2-antiplasmin | P08697 | 0.0000 | 0.4545 | Up | N/A |
| OS = Homo sapiens | | | | | |
| GN = SERPINF2 PE = 1 | | | | | |
| SV = 3 | | | | | |
| Junctional adhesion | Q9Y624 | 0.0000 | 0.4545 | Up | 14.16667 |
| molecule A OS = Homo | | | | | |
| sapiens GN = F11R PE = 1 | | | | | |
| SV = 1 | | | | | |
| C-reactive protein | P02741 | 0.0000 | 0.4545 | Up | 17.77778 |
| OS = Homo sapiens | | | | | |
| GN = CRP PE = 1 SV = 1 | | | | | |
| Complement factor H | P08603 | 0.0000 | 0.4545 | Up | N/A |
| OS = Homo sapiens | | | | | |
| GN = CFH PE = 1 SV = 4 | | | | | |
| Dermokine OS = Homo | Q6E0U4 | 0.0000 | 0.4545 | Up | 13.84615 |
| sapiens GN = DMKN PE = 1 | | | | | |
| SV = 3 | | | | | |
| Intercellular adhesion | P13598 | 0.0000 | 0.4545 | Up | N/A |
| molecule 2 OS = Homo | | | | | |
| sapiens GN = ICAM2 PE = 1 | | | | | |
| SV = 2 | | | | | |
| Protein NOV homolog | P48745 | 0.0000 | 0.4545 | Up | 12.92735 |
| OS = Homo sapiens | | | | | |
| GN = NOV PE = 1 SV = 1 | | | | | |
| Connective tissue growth | P29279 | 0.0000 | 0.4545 | Up | N/A |
| factor OS = Homo sapiens | | | | | |
| GN = CTGF PE = 1 SV = 2 | | | | | |
| Inter-alpha-trypsin inhibitor | P19823 | 0.0000 | 0.4545 | Up | N/A |
| heavy chain H2 OS = Homo | | | | | |
| sapiens GN = ITIH2 PE = 1 | | | | | |
| SV = 2 | | | | | |
| Protein S100-A6 OS = Homo | P06703 | 0.0000 | 0.4545 | Up | 7.323232 |
| sapiens GN = S100A6 PE = 1 | | | | | |
| SV = 1 | | | | | |
| Alpha-hemoglobin- | Q9NZD4 | 0.0000 | 0.4545 | Up | N/A |
| stabilizing protein | | | | | |
| OS = Homo sapiens | | | | | |
| GN = AHSP PE = 1 SV = 1 | | | | | |
| Complement factor H- | P36980 | 0.0000 | 0.4545 | Up | N/A |
| related protein 2 OS = Homo | | | | | |
| sapiens GN = CFHR2 PE = 1 | | | | | |
| SV = 1 | | | | | |
| Insulin-like growth factor- | P22692 | 0.0000 | 0.4545 | Up | N/A |
| binding protein 4 OS = Homo | | | | | |
| sapiens GN = IGFBP4 PE = 1 | | | | | |
| SV = 2 | | | | | |
| Macrophage colony- | P09603 | 0.0000 | 0.4697 | Up | 2.167549 |
| stimulating factor 1 | | | | | |
| OS = Homo sapiens | | | | | |
| GN = CSF1 PE = 1 SV = 2 | | | | | |
| Complement component C7 | P10643 | 0.0000 | 0.4805 | Up | 5.991637 |
| OS = Homo sapiens GN = C7 | | | | | |
| PE = 1 SV = 2 | | | | | |
| Cadherin-13 OS = Homo | P55290 | 0.8855 | 0.4827 | Up | 1.293557 |
| sapiens GN = CDH13 PE = 1 | | | | | |
| SV = 1 | | | | | |
| Ig kappa chain V-I region | P01598 | 0.8855 | 0.4848 | Up | 1.779417 |
| EU OS = Homo sapiens | | | | | |
| PE = 1 SV = 1 | | | | | |
| Ig alpha-2 chain C region | P01877 | 0.8855 | 0.4848 | Up | 2.589363 |
| OS = Homo sapiens | | | | | |
| GN = IGHA2 PE = 1 SV = 3 | | | | | |
| Immunoglobulin J chain | P01591 | 0.0000 | 0.4978 | Up | 1.102221 |
| OS = Homo sapiens GN = IGJ | | | | | |
| PE = 1 SV = 4 | | | | | |
| Guanylin OS = Homo sapiens | Q02747 | 2.6565 | 0.5087 | Up | 1.503226 |
| GN = GUCA2A PE = 1 SV = 2 | | | | | |
| Thrombospondin-1 | P07996 | 2.6565 | 0.5455 | Up | 1.822736 |
| OS = Homo sapiens | | | | | |
| GN = THBS1 PE = 1 SV = 2 | | | | | |
| Endothelial protein C | Q9UNN8 | 1.7710 | 0.5455 | Up | 1.695338 |
| receptor OS = Homo sapiens | | | | | |
| GN = PROCR PE = 1 SV = 1 | | | | | |
| Fibulin-1 OS = Homo sapiens | P23142 | 0.0000 | 0.5455 | Up | 3.520024 |
| GN = FBLN1 PE = 1 SV = 4 | | | | | |
| SPARC-like protein 1 | Q14515 | 0.0000 | 0.5455 | Up | 3.04518 |
| OS = Homo sapiens | | | | | |
| GN = SPARCL1 PE = 1 SV = 2 | | | | | |
| Nuclear transport factor 2 | P61970 | 0.0000 | 0.5455 | Up | 1.953211 |
| OS = Homo sapiens | | | | | |
| GN = NUTF2 PE = 1 SV = 1 | | | | | |
| L-lactate dehydrogenase B | P07195 | 0.0000 | 0.5455 | Up | 2.585354 |
| chain OS = Homo sapiens | | | | | |
| GN = LDHB PE = 1 SV = 2 | | | | | |
| Lithostathine-1-beta | P48304 | 0.0000 | 0.5541 | Up | 1.247021 |
| OS = Homo sapiens | | | | | |
| GN = REG1B PE = 1 SV = 1 | | | | | |
| Haptoglobin OS = Homo | P00738 | 0.0000 | 0.5887 | Up | 1.160193 |
| sapiens GN = HP PE = 1 SV = 1 | | | | | |
| Nidogen-1 OS = Homo | P14543 | 2.6565 | 0.5887 | Up | 1.30681 |
| sapiens GN = NID1 PE = 1 | | | | | |
| SV = 3 | | | | | |
| Tumor necrosis factor | Q9NP84 | 0.0000 | 0.5887 | Up | 6.115031 |
| receptor superfamily | | | | | |
| member 12A OS = Homo | | | | | |
| sapiens GN = TNFRSF12A | | | | | |
| PE = 1 SV = 1 | | | | | |
| Proactivator polypeptide | P07602 | 0.8855 | 0.5887 | Up | 1.85649 |
| OS = Homo sapiens | | | | | |
| GN = PSAP PE = 1 SV = 2 | | | | | |
| 6-phosphogluconolactonase | O95336 | 0.0000 | 0.6061 | Up | 2.381904 |
| OS = Homo sapiens | | | | | |
| GN = PGLS PE = 1 SV = 2 | | | | | |
| Ig lambda chain V-I region | P01700 | 0.0000 | 0.6061 | Up | 1.023292 |
| HA OS = Homo sapiens | | | | | |
| PE = 1 SV = 1 | | | | | |
| Fibulin-5 OS = Homo sapiens | Q9UBX5 | 1.7710 | 0.6234 | Up | 1.011263 |
| GN = FBLN5 PE = 1 SV = 1 | | | | | |
| Syndecan-1 OS = Homo | P18827 | 0.0000 | 0.6970 | Up | 2.852168 |
| sapiens GN = SDC1 PE = 1 | | | | | |
| SV = 3 | | | | | |
| Cathepsin L1 OS = Homo | P07711 | 0.0000 | 0.6970 | Up | 1.344818 |
| sapiens GN = CTSL1 PE = 1 | | | | | |
| SV = 2 | | | | | |
| Apolipoprotein D | P05090 | 1.7710 | 0.6991 | Up | 1.402984 |
| OS = Homo sapiens | | | | | |
| GN = APOD PE = 1 SV = 1 | | | | | |
| Kininogen-1 OS = Homo | P01042 | 15.9388 | 0.6991 | Up | 1.180075 |
| sapiens GN = KNG1 PE = 1 | | | | | |
| SV = 2 | | | | | |
| Prothrombin OS = Homo | P00734 | 0.8855 | 0.6991 | Up | 1.070533 |
| sapiens GN = F2 PE = 1 SV = 2 | | | | | |
| Ig kappa chain V-I region | P01593 | 12.3968 | 0.6991 | Up | 1.222718 |
| AG OS = Homo sapiens | | | | | |
| PE = 1 SV = 1 | | | | | |
| EGF-containing fibulin-like | Q12805 | 0.8855 | 0.6991 | Up | 1.932104 |
| extracellular matrix protein | | | | | |
| 1 OS = Homo sapiens | | | | | |
| GN = EFEMP1 PE = 1 SV = 2 | | | | | |
| Liver-expressed | Q969E1 | 7.9694 | 0.6991 | Up | 1.113239 |
| antimicrobial peptide 2 | | | | | |
| OS = Homo sapiens | | | | | |
| GN = LEAP2 PE = 1 SV = 1 | | | | | |
| Peptidase inhibitor 16 | Q6UXB8 | 0.0000 | 0.7078 | Up | 2.388318 |
| OS = Homo sapiens | | | | | |
| GN = PI16 PE = 1 SV = 1 | | | | | |
| Vascular cell adhesion | P19320 | 0.0000 | 0.7186 | Up | 1.647594 |
| protein 1 OS = Homo sapiens | | | | | |
| GN = VCAM1 PE = 1 SV = 1 | | | | | |
| Carbonic anhydrase 2 | P00918 | 0.0000 | 0.7273 | Up | 7.033249 |
| OS = Homo sapiens | | | | | |
| GN = CA2 PE = 1 SV = 2 | | | | | |
| Prostate-specific antigen | P07288 | 0.0000 | 0.7273 | Up | 2.09919 |
| OS = Homo sapiens | | | | | |
| GN = KLK3 PE = 1 SV = 2 | | | | | |
| Acyl-CoA-binding protein | P07108 | 0.0000 | 0.7273 | Up | 17.89474 |
| OS = Homo sapiens GN = DBI | | | | | |
| PE = 1 SV = 2 | | | | | |
| V-set and immunoglobulin | Q9Y279 | 0.0000 | 0.7273 | Up | 6.8 |
| domain-containing protein 4 | | | | | |
| OS = Homo sapiens | | | | | |
| GN = VSIG4 PE = 1 SV = 1 | | | | | |
| Lumican OS = Homo sapiens | P51884 | 0.0000 | 0.7273 | Up | 2.489899 |
| GN = LUM PE = 1 SV = 2 | | | | | |
| ADM OS = Homo sapiens | P35318 | 0.0000 | 0.7273 | Up | 2.794737 |
| GN = ADM PE = 1 SV = 1 | | | | | |
| Tyrosine-protein | P78324 (+1) | 0.0000 | 0.7273 | Up | 2.068376 |
| phosphatase non-receptor | | | | | |
| type substrate 1 OS = Homo | | | | | |
| sapiens GN = SIRPA PE = 1 | | | | | |
| SV = 2 | | | | | |
| Lymphatic vessel | Q9Y5Y7 | 0.0000 | 0.7294 | Up | 1.136625 |
| endothelial hyaluronic acid | | | | | |
| receptor 1 OS = Homo | | | | | |
| sapiens GN = LYVE1 PE = 1 | | | | | |
| SV = 2 | | | | | |
| Actin, cytoplasmic 1 | P60709 (+1) | 0.0000 | 0.7381 | Up | 1.000283 |
| OS = Homo sapiens | | | | | |
| GN = ACTB PE = 1 SV = 1 | | | | | |
| Vitronectin OS = Homo | P04004 | 0.8855 | 0.7381 | Up | 1.071916 |
| sapiens GN = VTN PE = 1 | | | | | |
| SV = 1 | | | | | |
| Complement decay- | P08174 | 0.0000 | 0.7403 | Up | 1.187707 |
| accelerating factor | | | | | |
| OS = Homo sapiens | | | | | |
| GN = CD55 PE = 1 SV = 4 | | | | | |
| Lithostathine-1-alpha | P05451 | 0.0000 | 0.7771 | Up | 1.1406 |
| OS = Homo sapiens | | | | | |
| GN = REG1A PE = 1 SV = 3 | | | | | |
| Plasminogen OS = Homo | P00747 | 0.0000 | 0.7965 | Up | 5.146586 |
| sapiens GN = PLG PE = 1 | | | | | |
| SV = 2 | | | | | |
| Glyceraldehyde-3-phosphate | P04406 | 0.0000 | 0.8485 | Up | 3.687516 |
| dehydrogenase OS = Homo | | | | | |
| sapiens GN = GAPDH PE = 1 | | | | | |
| SV = 3 | | | | | |
| Tripeptidyl-peptidase 1 | O14773 | 0.0000 | 0.8485 | Up | 1.238756 |
| OS = Homo sapiens | | | | | |
| GN = TPP1 PE = 1 SV = 2 | | | | | |
| Complement factor I | P05156 | 0.0000 | 0.8485 | Up | 1.704545 |
| OS = Homo sapiens GN = CFI | | | | | |
| PE = 1 SV = 2 | | | | | |
| Insulin-like growth factor- | P17936 | 0.0000 | 0.8485 | Up | 2.077333 |
| binding protein 3 OS = Homo | | | | | |
| sapiens GN = IGFBP3 PE = 1 | | | | | |
| SV = 2 | | | | | |
| Hemoglobin subunit | P69891 (+1) | 0.0000 | 0.8485 | Up | 6.760721 |
| gamma-1 OS = Homo sapiens | | | | | |
| GN = HBG1 PE = 1 SV = 2 | | | | | |
| WNT1-inducible-signaling | O76076 | 0.0000 | 0.8485 | Up | 1.410782 |
| pathway protein 2 | | | | | |
| OS = Homo sapiens | | | | | |
| GN = WISP2 PE = 1 SV = 1 | | | | | |
| Lactotransferrin OS = Homo | P02788 | 1.7710 | 0.8701 | Up | 1.538442 |
| sapiens GN = LTF PE = 1 | | | | | |
| SV = 6 | | | | | |
| Endonuclease domain- | O94919 | 0.0000 | 0.9242 | Up | 1.923709 |
| containing 1 protein | | | | | |
| OS = Homo sapiens | | | | | |
| GN = ENDOD1 PE = 1 SV = 2 | | | | | |
| Secretogranin-1 OS = Homo | P05060 | 1.7710 | 0.9242 | Up | 1.243701 |
| sapiens GN = CHGB PE = 1 | | | | | |
| SV = 2 | | | | | |
| Hemoglobin subunit beta | P68871 | 4.4274 | 0.9372 | Up | 2.991651 |
| OS = Homo sapiens | | | | | |
| GN = HBB PE = 1 SV = 2 | | | | | |
| Hemoglobin subunit alpha | P69905 | 1.7710 | 0.9372 | Up | 3.507837 |
| OS = Homo sapiens | | | | | |
| GN = HBA1 PE = 1 SV = 2 | | | | | |
| Gelsolin OS = Homo sapiens | P06396 | 4.4274 | 0.9372 | Up | 1.171937 |
| GN = GSN PE = 1 SV = 1 | | | | | |
| Cathepsin D OS = Homo | P07339 | 0.8855 | 0.9372 | Up | 1.015503 |
| sapiens GN = CTSD PE = 1 | | | | | |
| SV = 1 | | | | | |
| Lysosomal alpha- | P10253 | 1.7710 | 0.9372 | Up | 1.001588 |
| glucosidase OS = Homo | | | | | |
| sapiens GN = GAA PE = 1 | | | | | |
| SV = 4 | | | | | |
| Latent-transforming growth | Q14767 | 0.0000 | 0.9567 | Up | 2.420837 |
| factor beta-binding protein 2 | | | | | |
| OS = Homo sapiens | | | | | |
| GN = LTBP2 PE = 1 SV = 3 | | | | | |
| Collagen alpha-3(VI) chain | P12111 | 0.0000 | 0.9719 | Up | 1.604279 |
| OS = Homo sapiens | | | | | |
| GN = COL6A3 PE = 1 SV = 5 | | | | | |
| Collagen alpha-1(I) chain | P02452 | 3.5420 | 0.9740 | Up | 1.008726 |
| OS = Homo sapiens | | | | | |
| GN = COL1A1 PE = 1 SV = 5 | | | | | |
| Hemoglobin subunit delta | P02042 | 1.7710 | 0.9740 | Up | 3.554962 |
| OS = Homo sapiens | | | | | |
| GN = HBD PE = 1 SV = 2 | | | | | |
| Latent-transforming growth | Q14766 | 0.0000 | 0.9784 | Up | 1.056219 |
| factor beta-binding protein 1 | | | | | |
| OS = Homo sapiens | | | | | |
| GN = LTBP1 PE = 1 SV = 4 | | | | | |
| Alpha-amylase 1 OS = Homo | P04745 | 0.0000 | 1.0000 | Up | 3.69284 |
| sapiens GN = AMY1A PE = 1 | | | | | |
| SV = 2 | | | | | |
| Insulin-like growth factor- | Q16270 | 3.5420 | 1.0000 | Up | 1.133649 |
| binding protein 7 OS = Homo | | | | | |
| sapiens GN = IGFBP7 PE = 1 | | | | | |
| SV = 1 | | | | | |
| Semenogelin-2 OS = Homo | Q02383 | 0.0000 | 1.0000 | Up | N/A |
| sapiens GN = SEMG2 PE = 1 | | | | | |
| SV = 1 | | | | | |
| Granulins OS = Homo | P28799 | 0.8855 | 1.0000 | Up | 1.145038 |
| sapiens GN = GRN PE = 1 | | | | | |
| SV = 2 | | | | | |
| Fibronectin OS = Homo | P02751 | 0.0000 | 1.0000 | Up | 3.099908 |
| sapiens GN = FN1 PE = 1 | | | | | |
| SV = 4 | | | | | |
| Vasorin OS = Homo sapiens | Q6EMK4 | 1.7710 | 1.0000 | Up | 1.358585 |
| GN = VASN PE = 1 SV = 1 | | | | | |
| Low-density lipoprotein | P98164 | 0.8855 | 1.0000 | Up | 1.6173 |
| receptor-related protein 2 | | | | | |
| OS = Homo sapiens | | | | | |
| GN = LRP2 PE = 1 SV = 3 | | | | | |
| Cathelicidin antimicrobial | P49913 | 0.0000 | 1.0000 | Up | 1.382212 |
| peptide OS = Homo sapiens | | | | | |
| GN = CAMP PE = 1 SV = 1 | | | | | |
| Mucin-like protein 1 | Q96DR8 | 0.0000 | 1.0000 | Up | 4.065827 |
| OS = Homo sapiens | | | | | |
| GN = MUCL1 PE = 1 SV = 1 | | | | | |
| Semenogelin-1 OS = Homo | P04279 | 0.0000 | 1.0000 | Up | N/A |
| sapiens GN = SEMG1 PE = 1 | | | | | |
| SV = 2 | | | | | |
| Deleted in malignant brain | Q9UGM3 | 0.0000 | 1.0000 | Up | N/A |
| tumors 1 protein OS = Homo | | | | | |
| sapiens GN = DMBT1 PE = 1 | | | | | |
| SV = 2 | | | | | |
| Pyruvate kinase isozymes | P14618 | 0.0000 | 1.0000 | Up | 4.25 |
| M1/M2 OS = Homo sapiens | | | | | |
| GN = PKM2 PE = 1 SV = 4 | | | | | |
| Tumor necrosis factor | P08138 | 0.0000 | 1.0000 | Up | 2 |
| receptor superfamily | | | | | |
| member 16 OS = Homo | | | | | |
| sapiens GN = NGFR PE = 1 | | | | | |
| SV = 1 | | | | | |
| Zymogen granule protein 16 | Q96DA0 | 0.0000 | 1.0000 | Up | 1.155702 |
| homolog B OS = Homo | | | | | |
| sapiens GN = ZG16B PE = 1 | | | | | |
| SV = 3 | | | | | |
| Calreticulin OS = Homo | P27797 | 0.0000 | 1.0000 | Up | N/A |
| sapiens GN = CALR PE = 1 | | | | | |
| SV = 1 | | | | | |
| Flavin reductase OS = Homo | P30043 | 0.0000 | 1.0000 | Up | 1.038889 |
| sapiens GN = BLVRB PE = 1 | | | | | |
| SV = 3 | | | | | |
| Bisphosphoglycerate mutase | P07738 | 0.0000 | 1.0000 | Up | 4.090909 |
| OS = Homo sapiens | | | | | |
| GN = BPGM PE = 1 SV = 2 | | | | | |
| Cadherin-related family | Q9BYE9 | 0.0000 | 1.0000 | Up | 1.057214 |
| member 2 OS = Homo | | | | | |
| sapiens GN = CDHR2 PE = 1 | | | | | |
| SV = 2 | | | | | |
| Cadherin-11 OS = Homo | P55287 | 0.0000 | 1.0000 | Up | 4.166667 |
| sapiens GN = CDH11 PE = 1 | | | | | |
| SV = 2 | | | | | |
| Prostatic acid phosphatase | P15309 | 0.0000 | 1.0000 | Up | 1.342105 |
| OS = Homo sapiens | | | | | |
| GN = ACPP PE = 1 SV = 3 | | | | | |
| Beta-microseminoprotein | P08118 | 0.0000 | 1.0000 | Up | 1.789474 |
| OS = Homo sapiens | | | | | |
| GN = MSMB PE = 1 SV = 1 | | | | | |
| Ubiquitin carboxyl-terminal | P40818 | 0.0000 | 1.0000 | Up | N/A |
| hydrolase 8 OS = Homo | | | | | |
| sapiens GN = USP8 PE = 1 | | | | | |
| SV = 1 | | | | | |
| Signal-regulatory protein | O00241 | 0.0000 | 1.0000 | Up | 1.442308 |
| beta-1 OS = Homo sapiens | | | | | |
| GN = SIRPB1 PE = 1 SV = 5 | | | | | |
| Chitinase-3-like protein 1 | P36222 | 0.0000 | 1.0000 | Up | N/A |
| OS = Homo sapiens | | | | | |
| GN = CHI3L1 PE = 1 SV = 2 | | | | | |
| Heparin cofactor 2 | P05546 | 0.0000 | 1.0000 | Up | N/A |
| OS = Homo sapiens | | | | | |
| GN = SERPIND1 PE = 1 | | | | | |
| SV = 3 | | | | | |
| Complement C2 OS = Homo | P06681 | 0.0000 | 1.0000 | Up | N/A |
| sapiens GN = C2 PE = 1 SV = 2 | | | | | |
| Aminoacylase-1 OS = Homo | Q03154 | 0.0000 | 1.0000 | Up | 1.416667 |
| sapiens GN = ACY1 PE = 1 | | | | | |
| SV = 1 | | | | | |
| Thioredoxin domain- | Q8NBS9 | 0.0000 | 1.0000 | Up | N/A |
| containing protein 5 | | | | | |
| OS = Homo sapiens | | | | | |
| GN = TXNDC5 PE = 1 SV = 2 | | | | | |
| Mesothelin OS = Homo | Q13421 | 0.0000 | 1.0000 | Up | N/A |
| sapiens GN = MSLN PE = 1 | | | | | |
| SV = 2 | | | | | |
| Glutaminyl-peptide | Q16769 | 0.0000 | 1.0000 | Up | N/A |
| cyclotransferase OS = Homo | | | | | |
| sapiens GN = QPCT PE = 1 | | | | | |
| SV = 1 | | | | | |
| Cystatin-S OS = Homo | P01036 (+1) | 0.0000 | 1.0000 | Up | N/A |
| sapiens GN = CST4 PE = 1 | | | | | |
| SV = 3 | | | | | |
| Di-N-acetylchitobiase | Q01459 | 0.0000 | 1.0000 | Up | N/A |
| OS = Homo sapiens | | | | | |
| GN = CTBS PE = 1 SV = 1 | | | | | |
| Matrix-remodeling- | Q9BRK3 | 0.0000 | 1.0000 | Up | N/A |
| associated protein 8 | | | | | |
| OS = Homo sapiens | | | | | |
| GN = MXRA8 PE = 1 SV = 1 | | | | | |
| Mucin-5B OS = Homo | Q9HC84 | 0.0000 | 1.0000 | Up | N/A |
| sapiens GN = MUC5B PE = 1 | | | | | |
| SV = 3 | | | | | |
| Copper transport protein | O00244 | 0.0000 | 1.0000 | Up | N/A |
| ATOX1 OS = Homo sapiens | | | | | |
| GN = ATOX1 PE = 1 SV = 1 | | | | | |
| CMRF35-like molecule 1 | Q8TDQ1 | 0.0000 | 1.0000 | Up | N/A |
| OS = Homo sapiens | | | | | |
| GN = CD300LF PE = 1 SV = 3 | | | | | |
| Neprilysin OS = Homo | P08473 | 0.0000 | 1.0000 | Up | N/A |
| sapiens GN = MME PE = 1 | | | | | |
| SV = 2 | | | | | |
| Cytosolic non-specific | Q96KP4 | 0.0000 | 1.0000 | Up | N/A |
| dipeptidase OS = Homo | | | | | |
| sapiens GN = CNDP2 PE = 1 | | | | | |
| SV = 2 | | | | | |
| Ephrin type-B receptor 4 | P54760 | 0.0000 | 1.0000 | Up | N/A |
| OS = Homo sapiens | | | | | |
| GN = EPHB4 PE = 1 SV = 2 | | | | | |
| Fructose-1,6-bisphosphatase | P09467 | 0.0000 | 1.0000 | Up | N/A |
| 1 OS = Homo sapiens | | | | | |
| GN = FBP1 PE = 1 SV = 5 | | | | | |
| Peroxiredoxin-6 OS = Homo | P30041 | 0.0000 | 1.0000 | Up | N/A |
| sapiens GN = PRDX6 PE = 1 | | | | | |
| SV = 3 | | | | | |
| 5′(3′)- | Q8TCD5 | 0.0000 | 1.0000 | Up | N/A |
| deoxyribonucleotidase, | | | | | |
| cytosolic type OS = Homo | | | | | |
| sapiens GN = NTSC PE = 1 | | | | | |
| SV = 2 | | | | | |
| Inter-alpha-trypsin inhibitor | P19827 | 0.0000 | 1.0000 | Up | N/A |
| heavy chain H1 OS = Homo | | | | | |
| sapiens GN = ITIH1 PE = 1 | | | | | |
| SV = 3 | | | | | |
| D-dopachrome | P30046 | 0.0000 | 1.0000 | Up | N/A |
| decarboxylase OS = Homo | | | | | |
| sapiens GN = DDT PE = 1 | | | | | |
| SV = 3 | | | | | |
| Collagen alpha-1(XV) chain | P39059 | 0.0000 | 1.0000 | Up | N/A |
| OS = Homo sapiens | | | | | |
| GN = COL15A1 PE = 1 SV = 2 | | | | | |
| Folate receptor gamma | P41439 | 0.0000 | 1.0000 | Up | N/A |
| OS = Homo sapiens | | | | | |
| GN = FOLR3 PE = 1 SV = 1 | | | | | |
| Elongation factor 1-alpha 1 | P68104 (+1) | 0.0000 | 1.0000 | Up | N/A |
| OS = Homo sapiens | | | | | |
| GN = EEF1A1 PE = 1 SV = 1 | | | | | |
| Gamma-glutamyl hydrolase | Q92820 | 0.0000 | 1.0000 | Up | N/A |
| OS = Homo sapiens | | | | | |
| GN = GGH PE = 1 SV = 2 | | | | | |
| Heat shock protein beta-11 | Q9Y547 | 0.0000 | 1.0000 | Up | N/A |
| OS = Homo sapiens | | | | | |
| GN = HSPB11 PE = 1 SV = 1 | | | | | |
| Trans-Golgi network | O43493 | 0.0000 | 1.0000 | Up | N/A |
| integral membrane protein 2 | | | | | |
| OS = Homo sapiens | | | | | |
| GN = TGOLN2 PE = 1 SV = 2 | | | | | |
| Tetratricopeptide repeat | Q96AE7 | 0.0000 | 1.0000 | Up | N/A |
| protein 17 OS = Homo | | | | | |
| sapiens GN = TTC17 PE = 1 | | | | | |
| SV = 1 | | | | | |
| Keratin, type II cytoskeletal | P13647 | 2.6565 | 0.0281 | Down | 3.47272 |
| 5 OS = Homo sapiens | | | | | |
| GN = KRT5 PE = 1 SV = 3 | | | | | |
| Secreted Ly-6/uPAR-related | P55000 | 1.7710 | 0.0346 | Down | 4.24082 |
| protein 1 OS = Homo sapiens | | | | | |
| GN = SLURP1 PE = 1 SV = 2 | | | | | |
| Non-secretory ribonuclease | P10153 | 7.0839 | 0.0411 | Down | 2.665204 |
| OS = Homo sapiens | | | | | |
| GN = RNASE2 PE = 1 SV = 2 | | | | | |
| Keratin, type II cytoskeletal | P04264 | 17.7098 | 0.0476 | Down | 1.901671 |
| 1 OS = Homo sapiens | | | | | |
| GN = KRT1 PE = 1 SV = 6 | | | | | |
| Leukocyte-associated | Q6GTX8 | 0.0000 | 0.0541 | Down | 3.510833 |
| immunoglobulin-like | | | | | |
| receptor 1 OS = Homo | | | | | |
| sapiens GN = LAIR1 PE = 1 | | | | | |
| SV = 1 | | | | | |
| Keratin, type I cytoskeletal | P13645 | 1.7710 | 0.0887 | Down | 3.108684 |
| 10 OS = Homo sapiens | | | | | |
| GN = KRT10 PE = 1 SV = 6 | | | | | |
| Keratin, type I cytoskeletal 9 | P35527 | 6.1984 | 0.0931 | Down | 2.180652 |
| OS = Homo sapiens | | | | | |
| GN = KRT9 PE = 1 SV = 3 | | | | | |
| Immunoglobulin | Q969P0 | 0.0000 | 0.1126 | Down | 7.826065 |
| superfamily member 8 | | | | | |
| OS = Homo sapiens | | | | | |
| GN = IGSF8 PE = 1 SV = 1 | | | | | |
| CD27 antigen OS = Homo | P26842 | 0.8855 | 0.1126 | Down | 2.178961 |
| sapiens GN = CD27 PE = 1 | | | | | |
| SV = 2 | | | | | |
| CD44 antigen OS = Homo | P16070 | 3.5420 | 0.1277 | Down | 2.249827 |
| sapiens GN = CD44 PE = 1 | | | | | |
| SV = 3 | | | | | |
| Extracellular sulfatase Sulf- | Q8IWU5 | 3.5420 | 0.1320 | Down | 3.082946 |
| 2 OS = Homo sapiens | | | | | |
| GN = SULF2 PE = 1 SV = 1 | | | | | |
| Keratin, type I cytoskeletal | P02533 | 0.0000 | 0.1818 | Down | 12.58222 |
| 14 OS = Homo sapiens | | | | | |
| GN = KRT14 PE = 1 SV = 4 | | | | | |
| Kallikrein-1 OS = Homo | P06870 | 0.0000 | 0.2100 | Down | 2.956948 |
| sapiens GN = KLK1 PE = 1 | | | | | |
| SV = 2 | | | | | |
| Pepsin A OS = Homo sapiens | P00790 | 17.7098 | 0.2251 | Down | 2.243235 |
| GN = PGA3 PE = 1 SV = 1 | | | | | |
| Keratin, type II cytoskeletal | P05787 | 0.8855 | 0.2251 | Down | 2.543175 |
| 8 OS = Homo sapiens | | | | | |
| GN = KRT8 PE = 1 SV = 7 | | | | | |
| CMRF35-like molecule 9 | Q6UXG3 | 0.0000 | 0.2424 | Down | 3.232411 |
| OS = Homo sapiens | | | | | |
| GN = CD300LG PE = 1 SV = 2 | | | | | |
| Cubilin OS = Homo sapiens | O60494 | 1.7710 | 0.2857 | Down | 1.629224 |
| GN = CUBN PE = 1 SV = 5 | | | | | |
| Tumor necrosis factor | Q92956 | 0.8855 | 0.3030 | Down | 3.608824 |
| receptor superfamily | | | | | |
| member 14 OS = Homo | | | | | |
| sapiens GN = TNFRSF14 | | | | | |
| PE = 1 SV = 3 | | | | | |
| Secreted and transmembrane | Q8WVN6 | 2.6565 | 0.3074 | Down | 2.139448 |
| protein 1 OS = Homo sapiens | | | | | |
| GN = SECTM1 PE = 1 SV = 2 | | | | | |
| Uromodulin OS = Homo | P07911 | 80.5795 | 0.3095 | Down | 1.685097 |
| sapiens GN = UMOD PE = 1 | | | | | |
| SV = 1 | | | | | |
| CD59 glycoprotein | P13987 | 18.5953 | 0.3095 | Down | 1.699034 |
| OS = Homo sapiens | | | | | |
| GN = CD59 PE = 1 SV = 1 | | | | | |
| Ig kappa chain V-III region | P01621 | 1.7710 | 0.3095 | Down | 1.521406 |
| NG9 (Fragment) OS = Homo | | | | | |
| sapiens PE = 1 SV = 1 | | | | | |
| Protein shisa-5 OS = Homo | Q8N114 | 0.0000 | 0.3723 | Down | 2.413409 |
| sapiens GN = SHISA5 PE = 2 | | | | | |
| SV = 1 | | | | | |
| Carboxypeptidase N subunit | P22792 | 0.0000 | 0.3723 | Down | 1.661136 |
| 2 OS = Homo sapiens | | | | | |
| GN = CPN2 PE = 1 SV = 3 | | | | | |
| Pro-epidermal growth factor | P01133 | 0.8855 | 0.3810 | Down | 1.17975 |
| OS = Homo sapiens | | | | | |
| GN = EGF PE = 1 SV = 2 | | | | | |
| Ig kappa chain V-IV region | P01625 | 3.5420 | 0.3874 | Down | 1.345282 |
| Len OS = Homo sapiens | | | | | |
| PE = 1 SV = 2 | | | | | |
| Keratin, type II cytoskeletal | P35908 | 7.9694 | 0.3939 | Down | 2.672658 |
| 2 epidermal OS = Homo | | | | | |
| sapiens GN = KRT2 PE = 1 | | | | | |
| SV = 2 | | | | | |
| Trefoil factor 1 OS = Homo | P04155 | 0.0000 | 0.4113 | Down | 1.709739 |
| sapiens GN = TFF1 PE = 1 | | | | | |
| SV = 1 | | | | | |
| Phosphoinositide-3-kinase- | Q96FE7 | 7.0839 | 0.4199 | Down | 1.040962 |
| interacting protein 1 | | | | | |
| OS = Homo sapiens | | | | | |
| GN = PIK3IP1 PE = 1 SV = 2 | | | | | |
| Platelet glycoprotein VI | Q9HCN6 | 0.0000 | 0.4242 | Down | 2.276791 |
| OS = Homo sapiens GN = GP6 | | | | | |
| PE = 1 SV = 4 | | | | | |
| Alpha-enolase OS = Homo | P06733 | 0.0000 | 0.4372 | Down | 1.229981 |
| sapiens GN = ENO1 PE = 1 | | | | | |
| SV = 2 | | | | | |
| Cathepsin B OS = Homo | P07858 | 0.0000 | 0.4502 | Down | 1.752718 |
| sapiens GN = CTSB PE = 1 | | | | | |
| SV = 3 | | | | | |
| Src substrate cortactin | Q14247 | 0.0000 | 0.4545 | Down | 5.781818 |
| OS = Homo sapiens | | | | | |
| GN = CTTN PE = 1 SV = 2 | | | | | |
| Matrix metalloproteinase-9 | P14780 | 0.0000 | 0.4545 | Down | N/A |
| OS = Homo sapiens | | | | | |
| GN = MMP9 PE = 1 SV = 3 | | | | | |
| Protein YIPF3 OS = Homo | Q9GZM5 | 0.8855 | 0.4827 | Down | 1.812431 |
| sapiens GN = YIPF3 PE = 1 | | | | | |
| SV = 1 | | | | | |
| Basement membrane- | P98160 | 10.6259 | 0.4848 | Down | 1.004872 |
| specific heparan sulfate | | | | | |
| proteoglycan core protein | | | | | |
| OS = Homo sapiens | | | | | |
| GN = HSPG2 PE = 1 SV = 4 | | | | | |
| Trefoil factor 2 OS = Homo | Q03403 | 0.0000 | 0.4978 | Down | 1.740908 |
| sapiens GN = TFF2 PE = 1 | | | | | |
| SV = 2 | | | | | |
| Ig kappa chain V-II region | P01616 | 1.7710 | 0.5130 | Down | 1.308379 |
| MIL OS = Homo sapiens | | | | | |
| PE = 1 SV = 1 | | | | | |
| Agrin OS = Homo sapiens | O00468 | 0.0000 | 0.5238 | Down | 1.855864 |
| GN = AGRN PE = 1 SV = 4 | | | | | |
| Growth/differentiation | Q99988 | 0.0000 | 0.5455 | Down | 1.01072 |
| factor 15 OS = Homo sapiens | | | | | |
| GN = GDF15 PE = 1 SV = 3 | | | | | |
| Biotinidase OS = Homo | P43251 | 0.0000 | 0.5455 | Down | 1.388503 |
| sapiens GN = BTD PE = 1 | | | | | |
| SV = 2 | | | | | |
| Amyloid beta A4 protein | P05067 | 0.8855 | 0.5455 | Down | 1.82 |
| OS = Homo sapiens | | | | | |
| GN = APP PE = 1 SV = 3 | | | | | |
| Ig kappa chain V-II region | P01617 | 5.3129 | 0.5541 | Down | 1.195941 |
| TEW OS = Homo sapiens | | | | | |
| PE = 1 SV = 1 | | | | | |
| Mannan-binding lectin | O00187 | 0.8855 | 0.5541 | Down | 1.111565 |
| serine protease 2 OS = Homo | | | | | |
| sapiens GN = MASP2 PE = 1 | | | | | |
| SV = 4 | | | | | |
| Aminopeptidase N | P15144 | 0.8855 | 0.5671 | Down | 1.742588 |
| OS = Homo sapiens | | | | | |
| GN = ANPEP PE = 1 SV = 4 | | | | | |
| Beta-defensin 1 OS = Homo | P60022 | 0.8855 | 0.5714 | Down | 1.941947 |
| sapiens GN = DEFB1 PE = 1 | | | | | |
| SV = 1 | | | | | |
| Cadherin-1 OS = Homo | P12830 | 2.6565 | 0.5823 | Down | 1.244658 |
| sapiens GN = CDH1 PE = 1 | | | | | |
| SV = 3 | | | | | |
| Acid ceramidase OS = Homo | Q13510 | 0.0000 | 0.5823 | Down | 1.232505 |
| sapiens GN = ASAH1 PE = 1 | | | | | |
| SV = 5 | | | | | |
| Galectin-3-binding protein | Q08380 | 7.0839 | 0.5887 | Down | 1.094983 |
| OS = Homo sapiens | | | | | |
| GN = LGALS3BP PE = 1 | | | | | |
| SV = 1 | | | | | |
| Maltase-glucoamylase, | O43451 | 2.6565 | 0.6212 | Down | 1.917391 |
| intestinal OS = Homo sapiens | | | | | |
| GN = MGAM PE = 1 SV = 5 | | | | | |
| Collagen alpha-1(VI) chain | P12109 | 0.0000 | 0.6970 | Down | 1.542682 |
| OS = Homo sapiens | | | | | |
| GN = COL6A1 PE = 1 SV = 3 | | | | | |
| Peroxiredoxin-2 OS = Homo | P32119 | 0.0000 | 0.6970 | Down | 2.076825 |
| sapiens GN = PRDX2 PE = 1 | | | | | |
| SV = 5 | | | | | |
| Tyrosine-protein kinase | P30530 | 0.0000 | 0.6970 | Down | 2.195894 |
| receptor UFO OS = Homo | | | | | |
| sapiens GN = AXL PE = 1 | | | | | |
| SV = 3 | | | | | |
| Osteopontin OS = Homo | P10451 | 0.0000 | 0.6991 | Down | 1.369456 |
| sapiens GN = SPP1 PE = 1 | | | | | |
| SV = 1 | | | | | |
| WAP four-disulfide core | Q14508 | 47.8164 | 0.6991 | Down | 1.052492 |
| domain protein 2 OS = Homo | | | | | |
| sapiens GN = WFDC2 PE = 1 | | | | | |
| SV = 2 | | | | | |
| Interleukin-18-binding | O95998 | 0.0000 | 0.7273 | Down | 9.54 |
| protein OS = Homo sapiens | | | | | |
| GN = IL18BP PE = 1 SV = 2 | | | | | |
| Colipase OS = Homo sapiens | P04118 | 0.0000 | 0.7273 | Down | 4.037037 |
| GN = CLPS PE = 1 SV = 2 | | | | | |
| Hepatitis A virus cellular | Q8TDQO | 0.0000 | 0.7273 | Down | 2.48 |
| receptor 2 OS = Homo | | | | | |
| sapiens GN = HAVCR2 | | | | | |
| PE = 1 SV = 3 | | | | | |
| Low affinity | P08637 | 0.0000 | 0.7273 | Down | 6.36 |
| immunoglobulin gamma Fc | | | | | |
| region receptor III-A | | | | | |
| OS = Homo sapiens | | | | | |
| GN = FCGR3A PE = 1 SV = 2 | | | | | |
| Hemicentin-1 OS = Homo | Q96RW7 | 0.0000 | 0.7273 | Down | 1.330909 |
| sapiens GN = HMCN1 PE = 1 | | | | | |
| SV = 2 | | | | | |
| Pappalysin-2 OS = Homo | Q9BXP8 | 0.0000 | 0.7273 | Down | 1.573333 |
| sapiens GN = PAPPA2 PE = 1 | | | | | |
| SV = 4 | | | | | |
| Titin OS = Homo sapiens | Q8WZ42 | 0.0000 | 0.8052 | Down | 1.044141 |
| GN = TTN PE = 1 SV = 2 | | | | | |
| Resistin OS = Homo sapiens | Q9HD89 | 0.0000 | 0.8139 | Down | 1.3332 |
| GN = RETN PE = 2 SV = 1 | | | | | |
| Polymeric immunoglobulin | P01833 | 41.6180 | 0.8182 | Down | 1.112704 |
| receptor OS = Homo sapiens | | | | | |
| GN = PIGR PE = 1 SV = 4 | | | | | |
| Ribonuclease pancreatic | P07998 | 5.3129 | 0.8182 | Down | 1.075241 |
| OS = Homo sapiens | | | | | |
| GN = RNASE1 PE = 1 SV = 4 | | | | | |
| Peptidoglycan recognition | O75594 | 1.7710 | 0.8485 | Down | 1.266885 |
| protein 1 OS = Homo sapiens | | | | | |
| GN = PGLYRP1 PE = 1 SV = 1 | | | | | |
| Plasma serine protease | P05154 | 0.0000 | 0.8485 | Down | 1.536004 |
| inhibitor OS = Homo sapiens | | | | | |
| GN = SERPINA5 PE = 1 | | | | | |
| SV = 3 | | | | | |
| Vitelline membrane outer | Q7Z5L0 | 0.0000 | 0.8571 | Down | 1.4267 |
| layer protein 1 homolog | | | | | |
| OS = Homo sapiens | | | | | |
| GN = VMO1 PE = 1 SV = 1 | | | | | |
| Desmocollin-2 OS = Homo | Q02487 | 0.0000 | 0.8701 | Down | 1.356549 |
| sapiens GN = DSC2 PE = 1 | | | | | |
| SV = 1 | | | | | |
| Fibrillin-1 OS = Homo | P35555 | 0.0000 | 0.9221 | Down | 1.009699 |
| sapiens GN = FBN1 PE = 1 | | | | | |
| SV = 3 | | | | | |
| Lipocalin-1 OS = Homo | P31025 | 0.0000 | 0.9242 | Down | 1.420863 |
| sapiens GN = LCN1 PE = 1 | | | | | |
| SV = 1 | | | | | |
| Dipeptidyl peptidase 1 | P53634 | 2.6565 | 0.9351 | Down | 1.102486 |
| OS = Homo sapiens | | | | | |
| GN = CTSC PE = 1 SV = 2 | | | | | |
| Ig kappa chain V-III region | P04207 | 1.7710 | 0.9372 | Down | 1.42272 |
| CLL OS = Homo sapiens | | | | | |
| PE = 1 SV = 2 | | | | | |
| Ig kappa chain V-III region | P18135 | 7.9694 | 0.9805 | Down | 1.125563 |
| HAH OS = Homo sapiens | | | | | |
| PE = 2 SV = 1 | | | | | |
| Proteoglycan 4 OS = Homo | Q92954 | 0.8855 | 0.9805 | Down | 1.085214 |
| sapiens GN = PRG4 PE = 1 | | | | | |
| SV = 2 | | | | | |
| Prostaglandin-H2 D- | P41222 | 64.6407 | 1.0000 | Down | 1.184922 |
| isomerase OS = Homo | | | | | |
| sapiens GN = PTGDS PE = 1 | | | | | |
| SV = 1 | | | | | |
| Cystatin-M OS = Homo | Q15828 | 2.6565 | 1.0000 | Down | 1.158953 |
| sapiens GN = CST6 PE = 1 | | | | | |
| SV = 1 | | | | | |
| Inter-alpha-trypsin inhibitor | Q14624 | 2.6565 | 1.0000 | Down | 1.444266 |
| heavy chain H4 OS = Homo | | | | | |
| sapiens GN = ITIH4 PE = 1 | | | | | |
| SV = 4 | | | | | |
| Interleukin-1 receptor | P18510 | 0.0000 | 1.0000 | Down | 1.016646 |
| antagonist protein | | | | | |
| OS = Homo sapiens | | | | | |
| GN = IL1RN PE = 1 SV = 1 | | | | | |
| Trefoil factor 3 OS = Homo | Q07654 | 0.0000 | 1.0000 | Down | 2.233832 |
| sapiens GN = TFF3 PE = 1 | | | | | |
| SV = 1 | | | | | |
| Hepcidin OS = Homo sapiens | P81172 | 0.0000 | 1.0000 | Down | 1.093131 |
| GN = HAMP PE = 1 SV = 2 | | | | | |
| Dipeptidyl peptidase 4 | P27487 | 0.0000 | 1.0000 | Down | N/A |
| OS = Homo sapiens | | | | | |
| GN = DPP4 PE = 1 SV = 2 | | | | | |
| Submaxillary gland | P02814 | 0.0000 | 1.0000 | Down | 1.078075 |
| androgen-regulated protein | | | | | |
| 3B OS = Homo sapiens | | | | | |
| GN = SMR3B PE = 1 SV = 2 | | | | | |
| Olfactomedin-4 OS = Homo | Q6UX06 | 0.0000 | 1.0000 | Down | 1.425882 |
| sapiens GN = OLFM4 PE = 1 | | | | | |
| SV = 1 | | | | | |
| Platelet factor 4 OS = Homo | P02776 (+1) | 0.0000 | 1.0000 | Down | 1.190393 |
| sapiens GN = PF4 PE = 1 | | | | | |
| SV = 2 | | | | | |
| Eukaryotic translation | P56537 | 0.0000 | 1.0000 | Down | 1.227273 |
| initiation factor 6 OS = Homo | | | | | |
| sapiens GN = EIF6 PE = 1 | | | | | |
| SV = 1 | | | | | |
| Poliovirus receptor-related | Q92692 | 0.0000 | 1.0000 | Down | 1.23403 |
| protein 2 OS = Homo sapiens | | | | | |
| GN = PVRL2 PE = 1 SV = 1 | | | | | |
| Glutamyl aminopeptidase | Q07075 | 0.0000 | 1.0000 | Down | N/A |
| OS = Homo sapiens | | | | | |
| GN = ENPEP PE = 1 SV = 3 | | | | | |
| Na(+)/H(+) exchange | O14745 | 0.8855 | 1.0000 | Down | 2.560016 |
| regulatory cofactor NHE- | | | | | |
| RF1 OS = Homo sapiens | | | | | |
| GN = SLC9A3R1 PE = 1 | | | | | |
| SV = 4 | | | | | |
| Lymphocyte antigen 6D | Q14210 | 0.0000 | 1.0000 | Down | 1.289256 |
| OS = Homo sapiens | | | | | |
| GN = LY6D PE = 1 SV = 1 | | | | | |
| Collagen alpha-1(XII) chain | Q99715 | 0.0000 | 1.0000 | Down | 2.88 |
| OS = Homo sapiens | | | | | |
| GN = COL12A1 PE = 1 SV = 2 | | | | | |
| Extracellular superoxide | P08294 | 0.0000 | 1.0000 | Down | 21.6 |
| dismutase [Cu—Zn] | | | | | |
| OS = Homo sapiens | | | | | |
| GN = SOD3 PE = 1 SV = 2 | | | | | |
| Bone marrow proteoglycan | P13727 | 0.0000 | 1.0000 | Down | 1.414286 |
| OS = Homo sapiens | | | | | |
| GN = PRG2 PE = 1 SV = 2 | | | | | |
| Arylsulfatase A OS = Homo | P15289 | 0.0000 | 1.0000 | Down | 1.44 |
| sapiens GN = ARSA PE = 1 | | | | | |
| SV = 3 | | | | | |
| Sodium/potassium- | P54710 | 0.0000 | 1.0000 | Down | 1.090909 |
| transporting ATPase subunit | | | | | |
| gamma OS = Homo sapiens | | | | | |
| GN = FXYD2 PE = 1 SV = 3 | | | | | |
| Tenascin-X OS = Homo | P22105 | 0.0000 | 1.0000 | Down | 1.027027 |
| sapiens GN = TNXB PE = 1 | | | | | |
| SV = 3 | | | | | |
| Folate receptor alpha | P15328 | 0.0000 | 1.0000 | Down | 2.117647 |
| OS = Homo sapiens | | | | | |
| GN = FOLR1 PE = 1 SV = 3 | | | | | |
| Histone H2B type 1-K | O60814 (+13) | 0.0000 | 1.0000 | Down | 1.2 |
| OS = Homo sapiens | | | | | |
| GN = HIST1H2BK PE = 1 | | | | | |
| SV = 3 | | | | | |
| G-protein coupled receptor | Q9NQ84 | 0.0000 | 1.0000 | Down | 1.058824 |
| family C group 5 member C | | | | | |
| OS = Homo sapiens | | | | | |
| GN = GPRC5C PE = 1 SV = 2 | | | | | |
| Gamma-interferon-inducible | P13284 | 0.0000 | 1.0000 | Down | 1.44 |
| lysosomal thiol reductase | | | | | |
| OS = Homo sapiens | | | | | |
| GN = IFI30 PE = 1 SV = 3 | | | | | |
| Syntenin-1 OS = Homo | O00560 | 0.0000 | 1.0000 | Down | 1.058824 |
| sapiens GN = SDCBP PE = 1 | | | | | |
| SV = 1 | | | | | |
| Myosin-Vb OS = Homo | Q9ULV0 | 0.0000 | 1.0000 | Down | N/A |
| sapiens GN = MYO5B PE = 1 | | | | | |
| SV = 3 | | | | | |
| Lysosomal acid phosphatase | P11117 | 0.0000 | 1.0000 | Down | N/A |
| OS = Homo sapiens | | | | | |
| GN = ACP2 PE = 1 SV = 3 | | | | | |

Verification of the Prognostic Ability of Urinary Angiotensinogen in RRT Study

The inventors measured urinary angiotensinogen by ELISA and verified its ability to predict outcomes in a larger set of patients who developed AKI after cardiac surgery (n=97). The patients were divided into three groups by maximum AKI severity using the AKIN classification system: AKIN stage 1 (n=59), AKIN stage 2 (n=19), and AKIN stage 3 (n=19). Patient characteristics are shown by group in Table 3. The inventors performed two analyses. In the first, the inventors used all 97 patients regardless of the severity of AKI at the time of urine collection. There were no differences among the groups with respect to the following potential confounders: gender, race, age, weight, use of intraoperative bypass, bypass time, pre-operative sCr, and type of surgery. Since an objective was to identify a prognostic biomarker among patients with mild AKI, the inventors performed a second analysis on patients who had not progressed beyond AKIN stage 1 at the time of sample collection (n=79). Grouping patients by maximum AKIN stage, there were no differences among the groups with respect to time of urine sample collection and sCr at the time of collection, in addition to the previously mentioned confounders.

TABLE 3
Characteristics of cardiac surgery patients used to verify the potential of urinary angiotensinogen as a biomarker of post-operative AKI

| | AKI of Any Stage at Time of Sample Collection | | | | AKIN Stage 1 at Time of Sample Collection | | | |
|---|---|---|---|---|---|---|---|---|
| | AKIN Stage 1 | AKIN Stage 2 | AKIN Stage 3 | p-value | AKIN Stage 1 | AKIN Stage 2 | AKIN Stage 3 | p-value |
| n | 59 | 19 | 19 | | 59 | 10 | 10 | |
| uAnCr | 22.6 | 34.1 | 58.8 | | 22.6 | 35.3 | 77.0 | |
| | (13.1-54.0) | (11.1-50.4) | (20.4-217.1) | 0.014 | (13.1-54.0) | (22.8-270.3) | (30.9-329.4) | 0.014 |
| Male | 71% (42) | 74% (14) | 68% (13) | 0.94 | 71% (42) | 60% (6) | 70% (7) | 0.78 |
| Caucasian | 64% (38) | 63% (12) | 79% (15) | 0.47 | 64% (38) | 60% (6) | 80% (8) | 0.58 |
| Age (yrs) | 65.8 +/− 10.8 | 84.5 +/− 10.0 | 68.5 +/− 11.9 | 0.53 | 65.8 +/− 10.8 | 68.2 +/− 10.8 | 89.0 +/− 14.7 | 0.58 |
| Weight (kg) | 88.2 +/− 24.3 | 94.41 +/− 23.8 | 88.9 +/− 27.3 | 0.83 | 88.2 +/− 24.3 | 84.7 +/− 21.7 | 88.1 +/− 33.3 | 0.69 |
| Bypass | 76% (35) | | | | 76% (35) | 80% (8) | 70% (7) | 0.88 |
| Bypass Time | 143.2 +/− 72.5 | 145.4 +/− 75.6 | 118.9 +/− 67.3 | 0.31 | 143.2 +/− 72.5 | 154.4 +/− 74.7 | 146.4 +/− 77.8 | 0.95 |
| (hrs) | | | | | | | | |
| Pre-op sCr | 1.2 +/− 0.3 | 1.2 +/− 0.4 | 1.2 +/− 0.5 | 0.19 | 1.2 +/− 0.3 | 1.2 +/− 0.4 | 1.4 +/− 0.5 | 0.74 |
| (mg/dl) | | | | | | | | |
| sCr at Collection | 1.7 +/− 0.4 | 2.0 +/− 0.7 | 2.5 +/− 0.8 | 0.001 | 1.7 +/− 0.4 | 1.7 +/− 0.6 | 2.3 +/− 0.8 | 0.14 |
| (mg/dl) | | | | | | | | |
| Collection Time | 27.9 +/− 11.8 | 31.6 +/− 14.5 | 36.0 +/− 11.0 | 0.04 | 27.9 +/− 11.8 | 26.2 +/− 15.8 | 35.2 +/− 12.2 | 0.23 |
| (post-op hrs) | | | | | | | | |
| Max sCr (mg/dl) | 1.9 +/− 0.4 | 2.7 +/− 0.8 | 4.0 +/− 1.9 | <0.001 | 1.9 +/− 0.4 | 2.8 +/− 0.8 | 4.3 +/− 2.6 | <0.001 |
| Days to Max sCr | 2.3 +/− 2.0 | 3.0 +/− 1.8 | 4.3 +/− 2.9 | 0.001 | 2.3 +/− 2.0 | 3.9 +/− 2.0 | 4.8 +/− 3.0 | <0.001 |
| (post-op) | | | | | | | | |
| RRT 10 days | 0 | 0 | 47% (9) | <0.001 | 0 | 0 | 80% (8) | <0.001 |
| Death | 0 | 11% (2) | 32% (6) | <0.001 | 0 | 20% (2) | 30% (3) | <0.001 |

Only patients who developed post-operative AKI within 48 hours after surgery were included in our study. We performed two separate analyses, one on the group as a whole, and one on the subset of patients who were AKIN Stage 1 at the time of sample collection. This latter analysis was done to test the hypothesis that angiotensinogen is predictive of outcomes at an early time point during the course of AKI. Continuous variables are reported as mean and standard deviation, except for uAnCr which is reported as median and interquartile range (units are ng of urinary angiotensinogen i mg of urine creatinine). Categorical variables are shown as percentage and number.

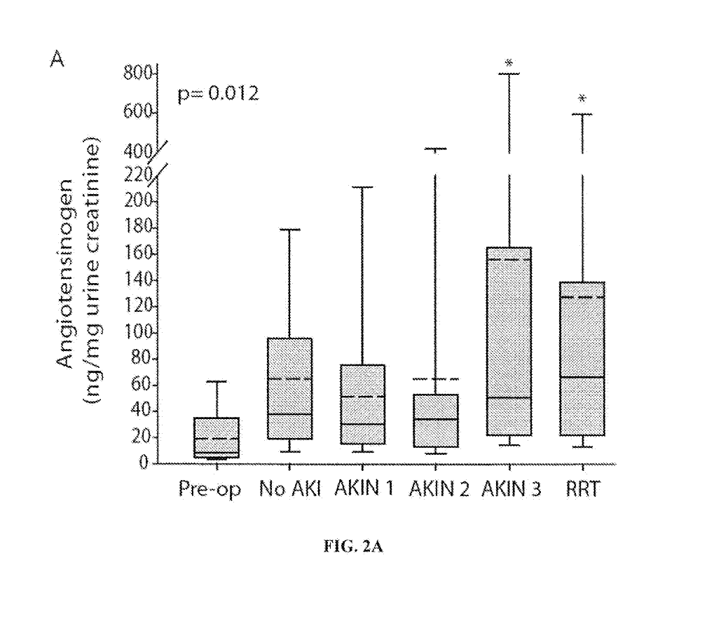

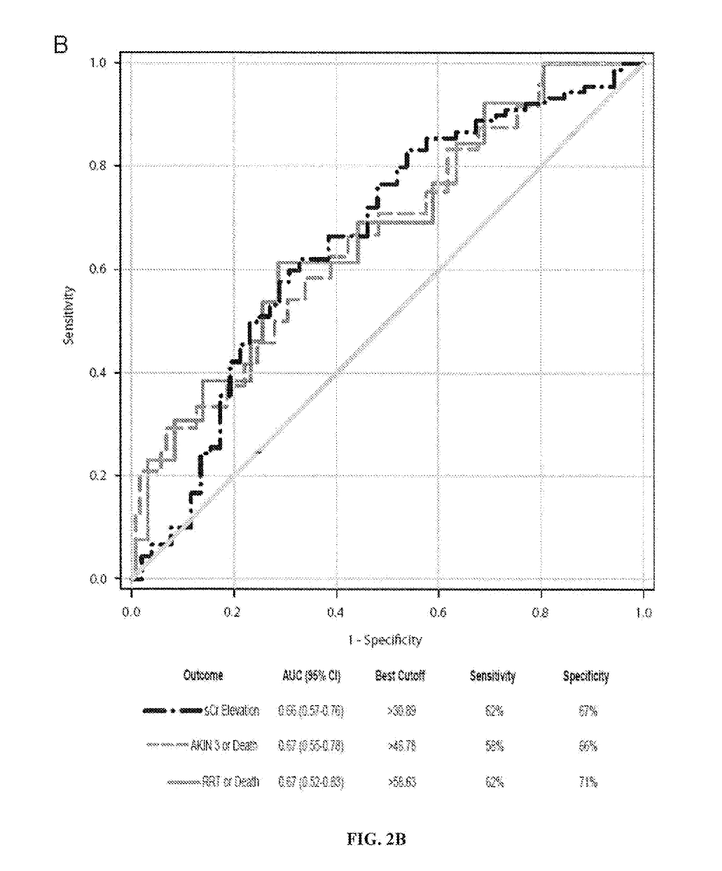

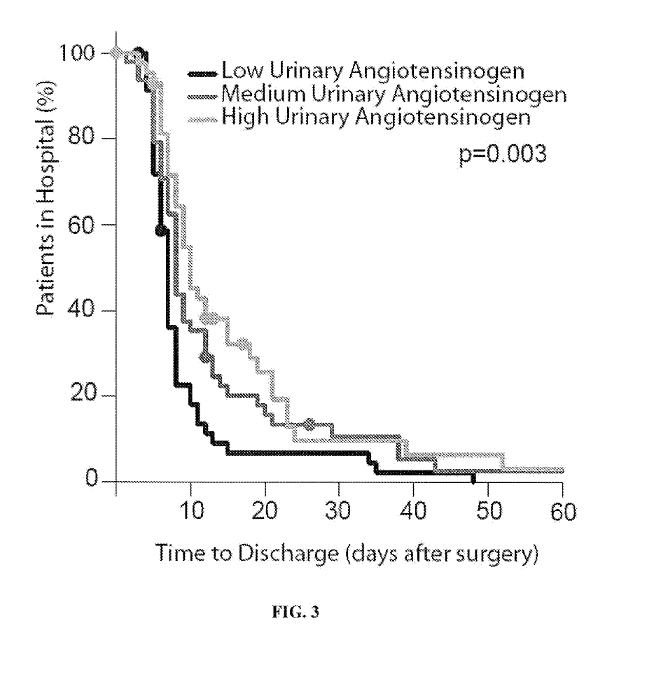

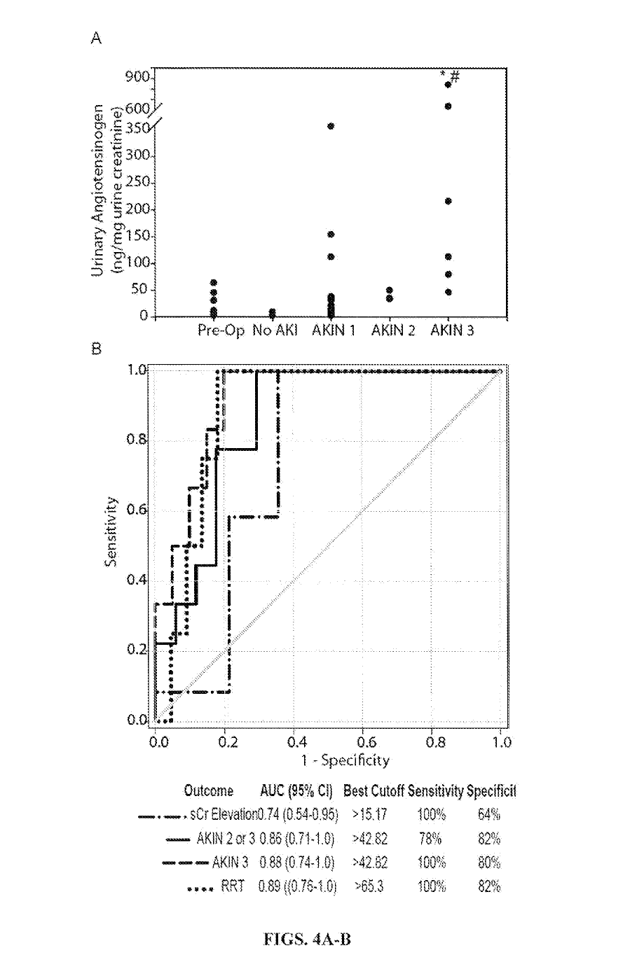

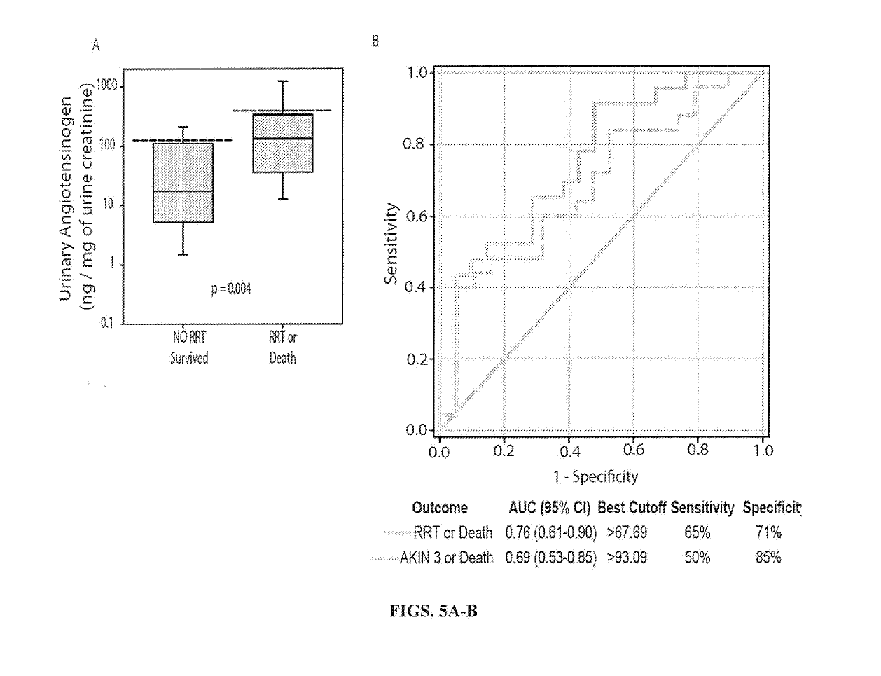

Among all AKI patients, urinary angiotensinogen corrected for urine creatinine (uAnCR; ng angiotensinogen/mg creatinine) was correlated with both maximum sCr (r=0.49; p<0.001) and maximum percent change in sCr (r=0.29; p=0.01), and there was a trend toward higher uAnCR in patients with increasingly severe AKI (FIG. 2A; Table 3). Pair-wise comparison revealed a significant difference between the AKIN stage 3 (median uAnCR 58.8 ng/mg) and AKIN stage 1 (median uAnCR 22.6 ng/mg) groups. The relationship between uAnCR and AKI severity was also observed in the subset of patients who were classified as AKIN stage 1 at sample collection (FIG. 2B; Table 3). These data suggest that uAnCR could have prognostic relevance at the time of diagnosis with AKI, even in cases where there is only a mild increase in sCr. Receiver operator characteristic (ROC) curves were used to evaluate the prognostic predictive power of uAnCR with respect to the primary outcomes of worsening of AKI (defined as progression to the next AKIN stage) and the need for renal replacement therapy (RRT) within 10 days. Several secondary outcomes were also tested, including development of AKIN stage 3, and the composite outcomes development of AKIN stage 2 or 3, AKIN stage 3 or death (defined as 30 day in-hospital mortality), and RRT or death. The test was considered predictive of the outcome if the AUC value was significantly different from an AUC of 0.5. Among all AKI patients, uAnCR was predictive of all tested outcomes (FIG. 3). Among patients classified as AKIN stage 1 at the time of collection, it was significantly predictive of all outcomes except for RRT, and its predictive power appeared to be slightly augmented in comparison to the previous analysis (FIG. 4). While the ROC curve for RRT prediction in these patients was not statistically significant (p=0.1), it is likely that it was underpowered since only eight patients required RRT in this group. Notably, uAnCR discriminated with high accuracy (AUC=0.81) between patients who later met the outcome of severe AKI (AKIN stage 3) or death and those who did not. In addition to the prediction of these outcomes, the inventors noted a relationship between uAnCR and length of stay. This relationship is visualized in survival curves plotting the time to discharge (defined as days after sample collection) of patients in the upper, middle or lower tertiles of uAnCR (high, med, and low uAnCR, respectively). Among all AKI patients and in the subset of patients having AKIN stage 1 at the time of collection, those patients with higher uAnCR concentrations had longer hospital stays (FIGS. 5A-B). ROC curve analysis indicated that lower uAnCR was predictive of length of stay (the outcome was defined as discharge≦7 days from the time of sample collection). Tables 4 and 5 summarize the performance characteristics of uAnCR as a predictor of the tested outcomes in patients who had AKI of any stage at the time of sample collection and those who had not progressed beyond AKIN stage 1 at the time of sample collection, respectively.

TABLE 4
Performance characteristics of uAnCR as a prognostic AKI biomarker among patients
who were any stage AKI at the time of sample collection (n = 97)

| Outcome | AUC | Cut-Off | | Sensitivity | Specificity | LR+ | LR− | PPV | NPV |
|---|---|---|---|---|---|---|---|---|---|
| Worsening AKI | 0.70 | Best | >38.27 ng/mg | 70.8% | 66.2% | 2.09 | 0.44 | 67.7% | 69.4% |
| | (0.57-0.82) | Max PPV | >392.5 ng/mg | 16.7% | 98.5% | 11.34 | 0.85 | 91.9% | 54.2% |
| | | Max NPV | >12.55 ng/mg | 91.7% | 26.5% | 1.25 | 0.31 | 55.5% | 76.1% |
| AKIN Stage 3 AKI | 0.71 | Best | >34.33 ng/mg | 68.4% | 65.4% | 1.98 | 0.48 | 66.4% | 67.4% |
| | (0.59-0.84) | Max PPV | >572.0 ng/mg | 15.8% | 98.7% | 12.34 | 0.85 | 92.5% | 54.0% |
| | | Max NPV | >14.06 ng/mg | 94.7% | 30.8% | 1.37 | 0.17 | 57.8% | 85.4% |
| RRT | 0.71 | Best | >58.63 ng/mg | 66.7% | 77.3% | 2.93 | 0.43 | 74.6% | 69.9% |
| | (0.54-0.88) | Max PPV | >572.0 ng/mg | 11.1% | 96.6% | 3.26 | 0.92 | 76.5% | 52.1% |
| | | Max NPV | >20.01 ng/mg | 88.9% | 40.9% | 1.50 | 0.27 | 60.1% | 78.6% |
| AKIN Stage 2 or 3 AKI | 0.64 | Best | >34.33 ng/mg | 57.9% | 69.5% | 1.90 | 0.61 | 65.5% | 62.3% |
| | (0.52-0.73) | Max PPV | >392.5 ng/mg | 13.2% | 98.3% | 7.79 | 0.88 | 88.6% | 53.1% |
| | | Max NPV | >6.777 ng/mg | 97.4% | 10.2% | 1.08 | 0.26 | 52.0% | 79.5% |
| AKIN 3 or Death | 0.75 | Best | >37.36 ng/mg | 66.7% | 72.4% | 2.41 | 0.46 | 70.7% | 68.5% |
| | (0.64-0.87) | Max PPV | >392.5 ng/mg | 23.8% | 98.7% | 18.04 | 0.77 | 94.7% | 56.4% |
| | | Max NPV | >14.06 ng/mg | 95.2% | 31.6% | 1.39 | 0.15 | 58.2% | 86.9% |
| RRT or Death | 0.71 | Best | >58.63 ng/mg | 61.5% | 78.6% | 2.87 | 0.49 | 74.2% | 67.1% |
| | (0.55-0.86) | Max PPV | >466.6 ng/mg | 23.1% | 97.6% | 9.70 | 0.79 | 90.7% | 55.9% |
| | | Max NPV | >16.28 ng/mg | 92.3% | 35.7% | 1.44 | 0.22 | 58.9% | 82.3% |
| Length of Stay | 0.74 | Best | <26.38 ng/mg | 68.3% | 69.6% | 2.25 | 0.46 | 69.2% | 68.7% |
| | (0.64-0.84) | Max PPV | <13.78 ng/mg | 43.9% | 89.3% | 4.10 | 0.63 | 80.4% | 61.4% |
| | | Max NPV | <109.0 ng/mg | 97.6% | 26.8% | 1.33 | 0.09 | 57.1% | 91.7% |

Three thresholds for each outcome are listed. The best threshold had the best balance of sensitivity and specificity of all the cut-offs in the dataset. The maximum PPV and NPV cut-offs were chosen to maximize the positive and negative likelihood ratios. While these cut-offs generally lack either high sensitivity or high specificity, they can be clinically useful for definitively assigning patients to high and low risk categories.

TABLE 5
Performance characteristics of uAnCR as a prognostic AKI biomarker among patients
who were classified as AKIN Stage 1 at the time of sample collection (n = 79)

| Outcome | AUC | Cut-Off | | Sensitivity | Specificity | LR+ | LR− | PPV | NPV |
|---|---|---|---|---|---|---|---|---|---|
| Worsening AKI | 0.71 | Best | >33.27 ng/mg | 75.0% | 66.1% | 2.21 | 0.38 | 68.9% | 72.6% |
| | (0.57-0.85) | Max PPV | >392.5 ng/mg | 20.0% | 98.3% | 11.83 | 0.81 | 92.2% | 55.1% |
| | | Max NPV | >19.95 ng/mg | 85.0% | 44.1% | 1.52 | 0.34 | 60.3% | 74.6% |
| AKIN Step 3 AKI | 0.75 | Best | >58.63 ng/mg | 70.0% | 78.3% | 3.22 | 0.38 | 76.3% | 72.3% |
| | (0.58-0.92) | Max PPV | >572.0 ng/mg | 20.0% | 98.6% | 13.79 | 0.81 | 93.2% | 55.2% |
| | | Max NPV | >19.95 ng/mg | 90.0% | 49.6% | 1.51 | 0.25 | 60.2% | 80.2% |
| RRT | 0.68 | Best | >34.33 ng/mg | 75.0% | 63.4% | 2.05 | 0.39 | 67.2% | 71.7% |
| | (0.49-0.87) | Max PPV | >572.0 ng/mg | 12.5% | 97.2% | 4.43 | 0.90 | 81.6% | 52.6% |
| | | Max NPV | >19.95 ng/mg | 87.5% | 39.4% | 1.44 | 0.32 | 59.1% | 75.9% |
| AKIN 3 or Death | 0.81 | Best | >58.63 ng/mg | 75.0% | 80.6% | 3.87 | 0.31 | 79.4% | 76.3% |
| | (0.66-0.95) | Max PPV | >392.5 ng/mg | 33.3% | 98.5% | 22.37 | 0.68 | 95.7% | 59.6% |
| | | Max NPV | >19.95 ng/mg | 91.7% | 41.8% | 1.57 | 0.20 | 61.2% | 83.4% |
| RRT or Death | 0.76 | Best | >58.63 ng/mg | 70.0% | 78.3% | 3.22 | 0.38 | 76.3% | 72.3% |
| | (0.59-0.93) | Max PPV | >466.6 ng/mg | 30.0% | 98.6% | 20.69 | 0.71 | 95.4% | 58.5% |
| | | Max NPV | >19.95 ng/mg | 90.0% | 40.6% | 1.51 | 0.25 | 60.2% | 80.2% |
| Length of Stay | 0.74 | Best | >26.38 ng/mg | 68.6% | 72.7% | 2.51 | 0.43 | 71.5% | 69.8% |
| | (0.63-0.85) | Max PPV | >4.395 ng/mg | 8.6% | 97.7% | 3.78 | 0.94 | 79.1% | 51.7% |
| | | Max NPV | >109.0 ng/mg | 97.1% | 27.3% | 1.34 | 0.10 | 57.2% | 90.5% |

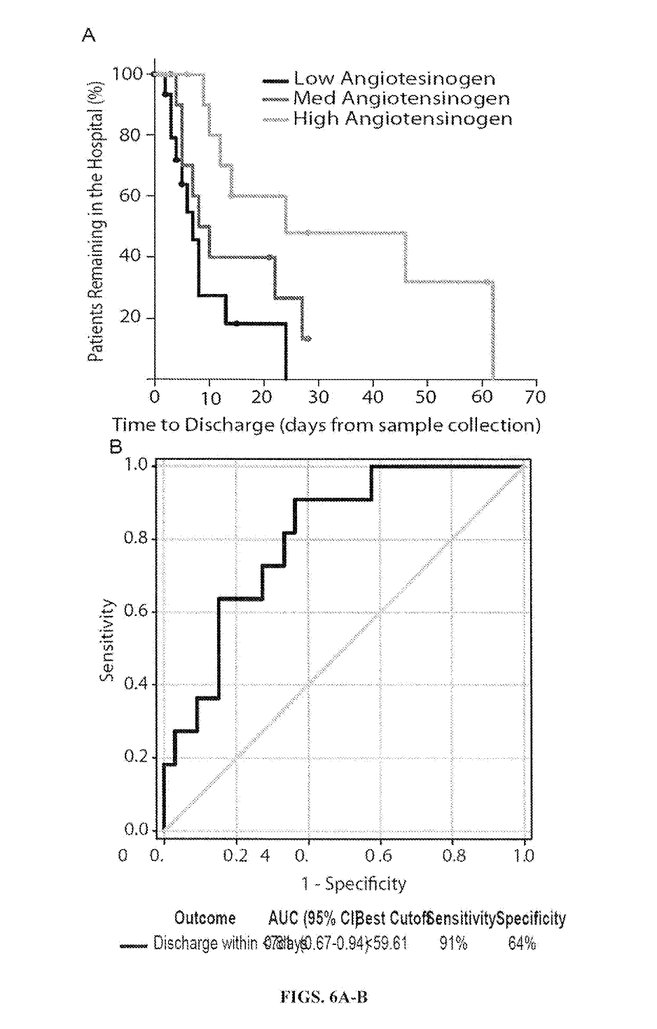

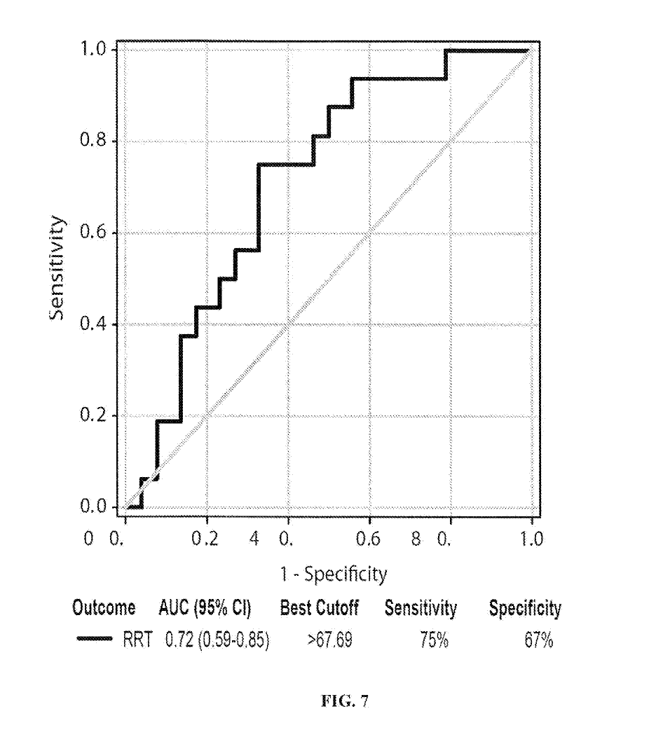

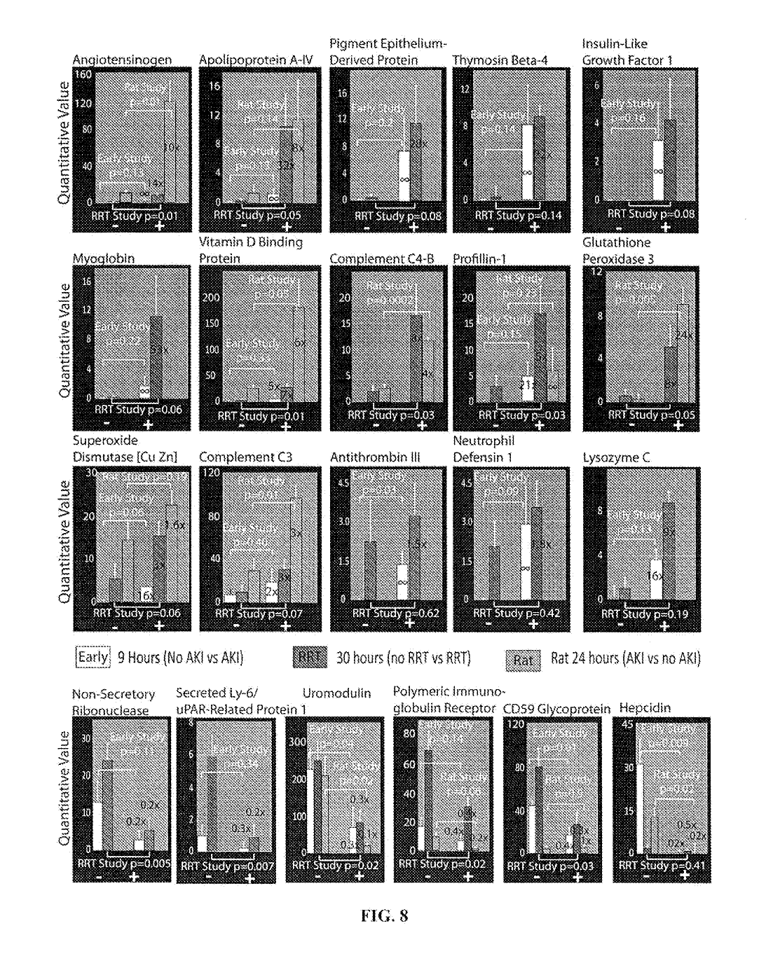

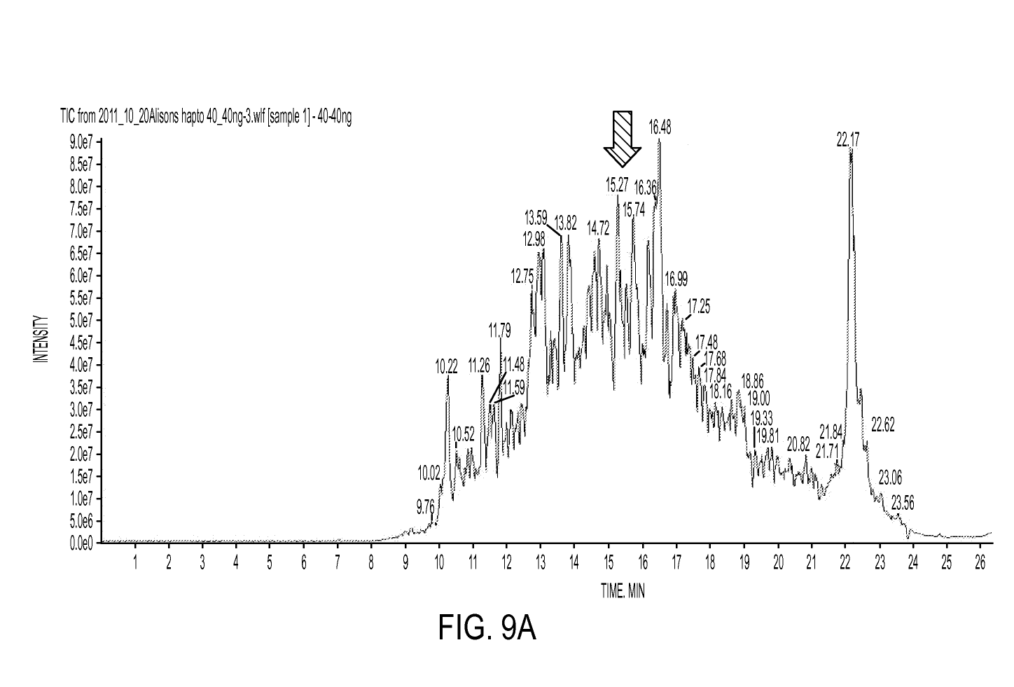

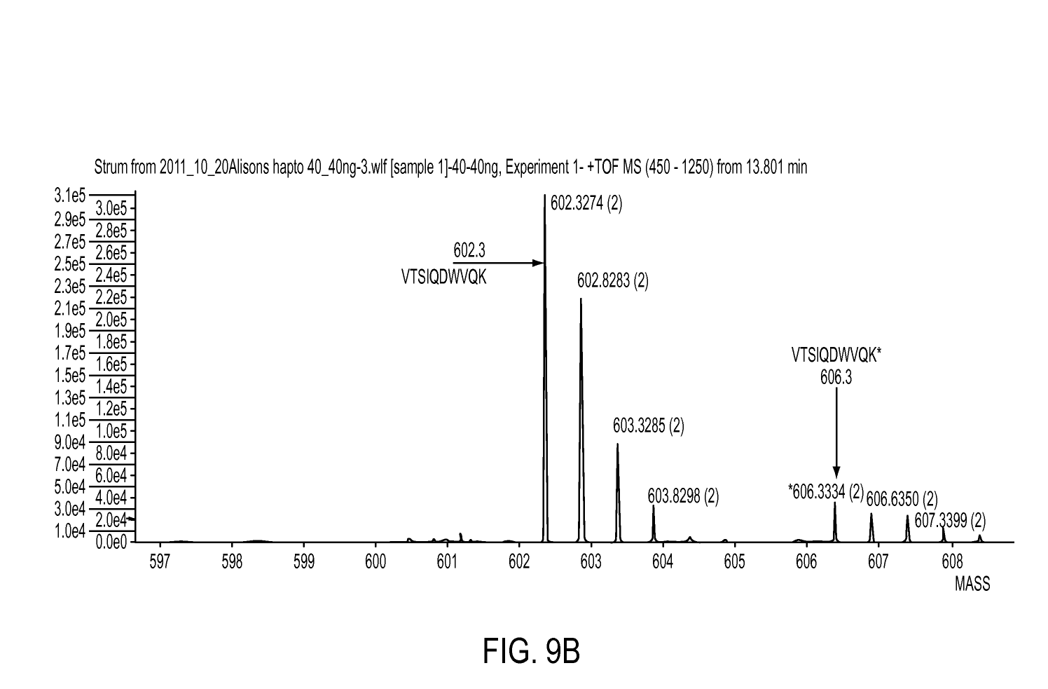

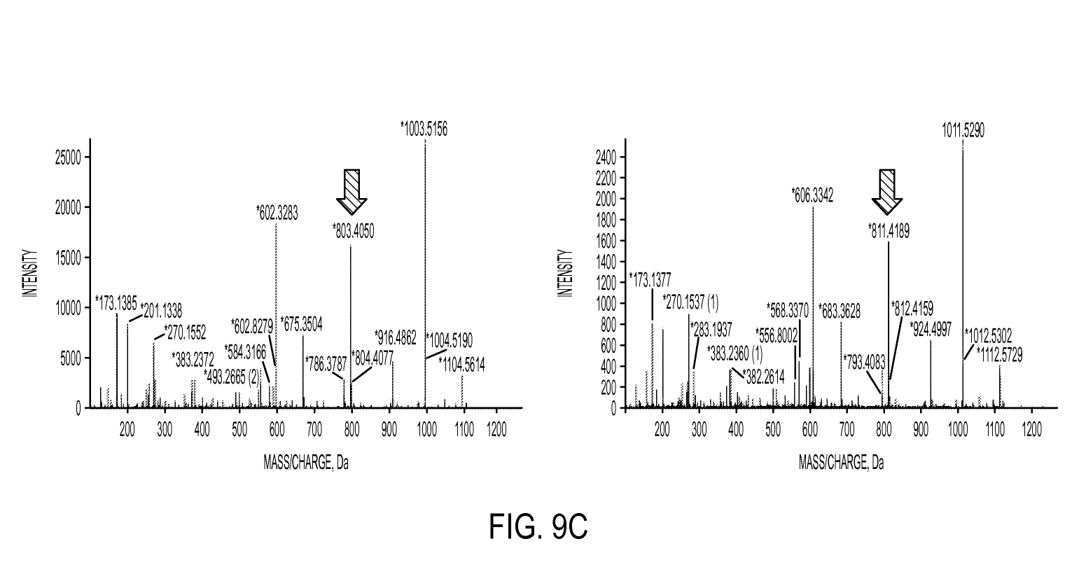

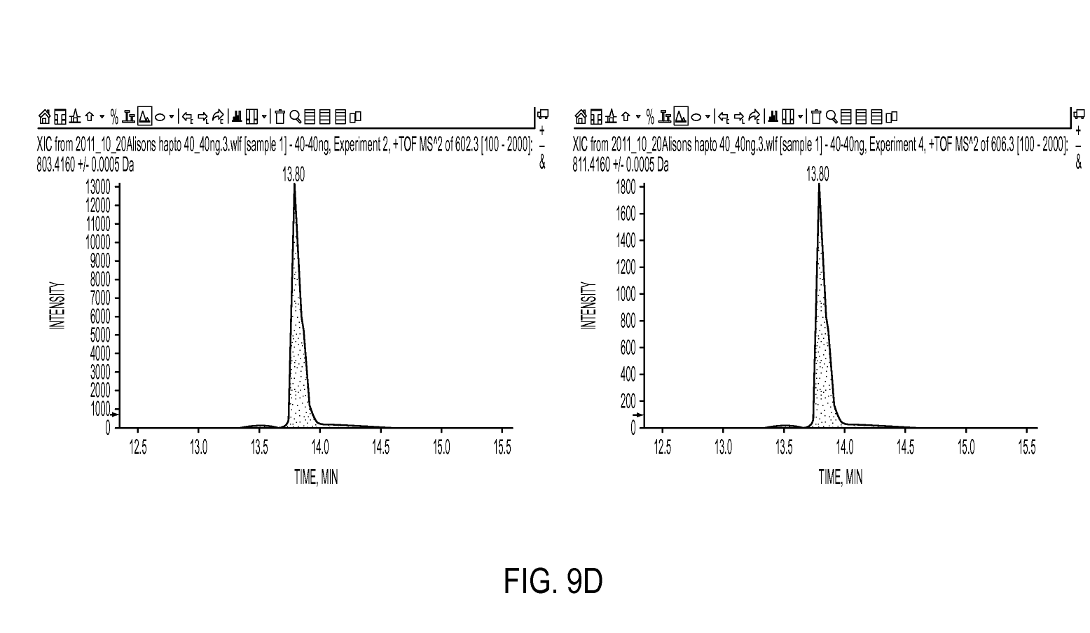

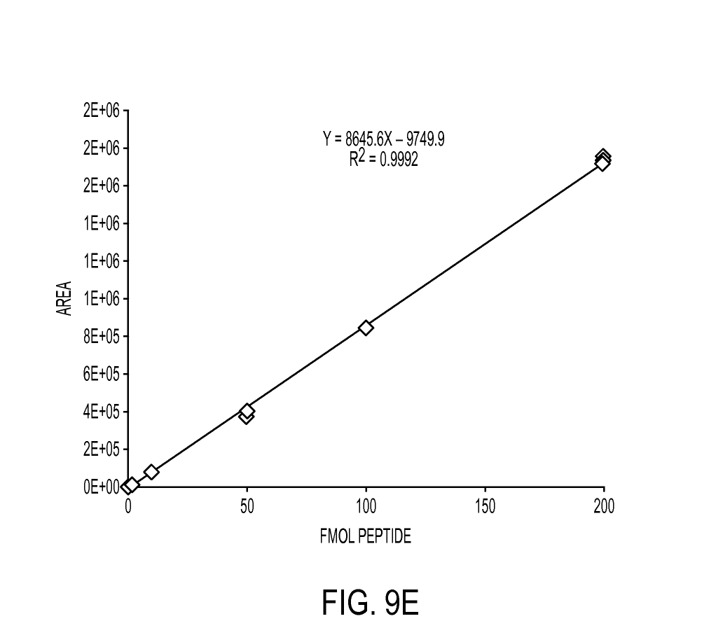

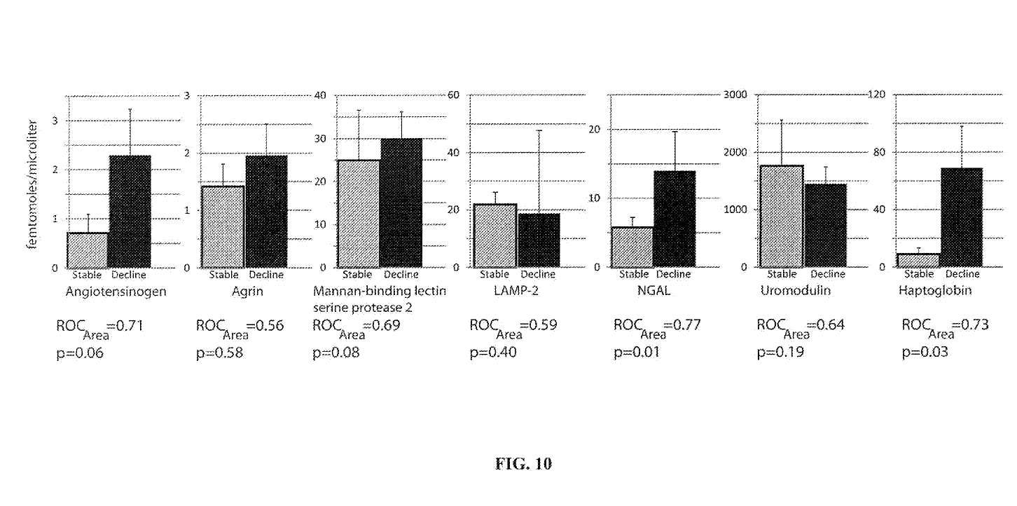

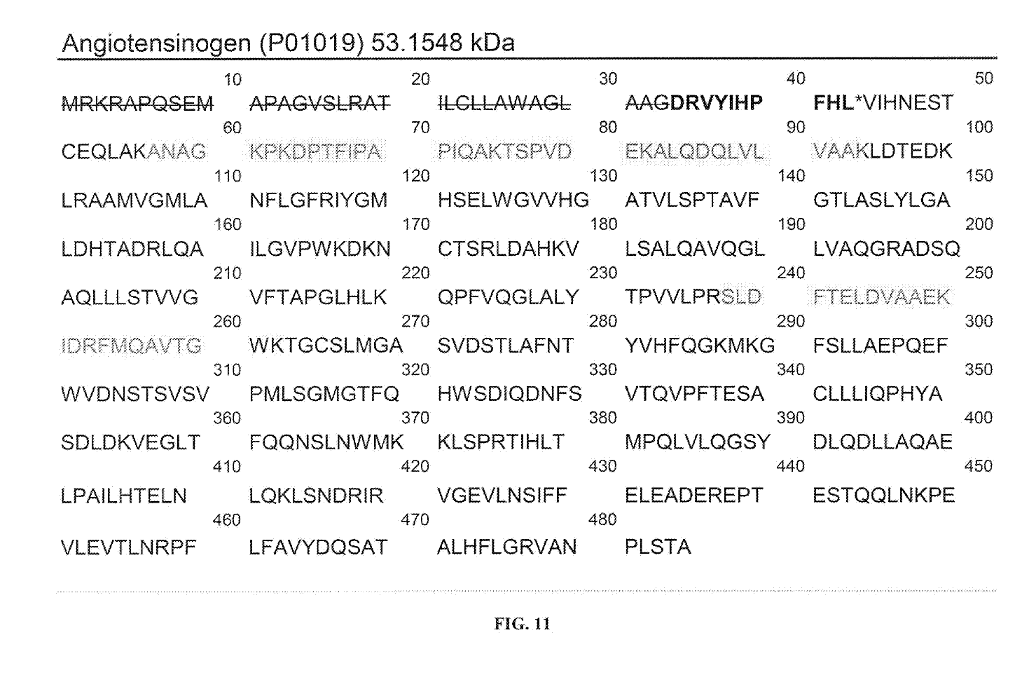

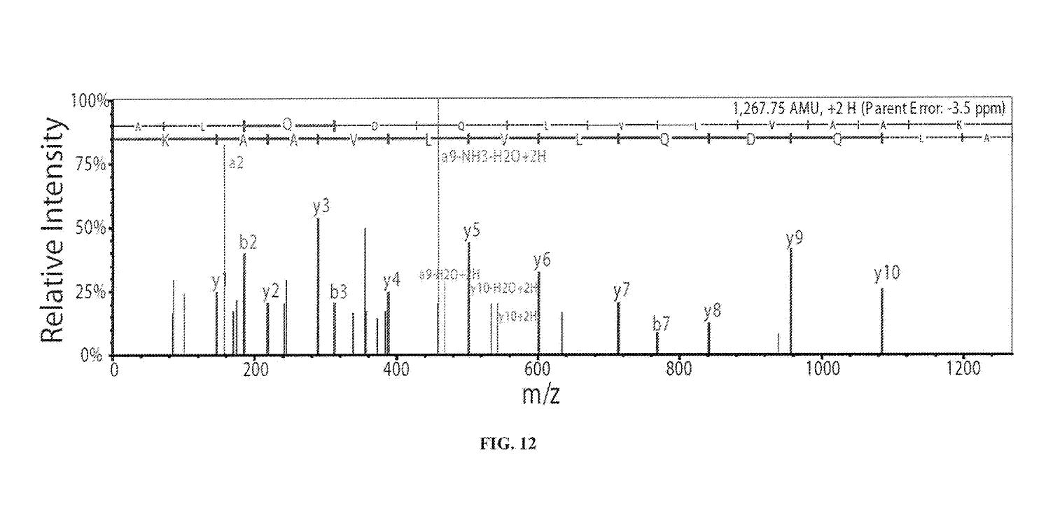

As a final analysis, the inventors evaluated the prognostic predictive power of uAnCR in the subset of twenty-two AKI patients (some of whom had advanced AKI at the time of sample collection) who had undergone off-pump cardiac surgery (that is without intraoperative cardiopulmonary bypass). The rationale for this analysis was due to the stronger correlation of uAnCR with maximum sCr (r=0.65; p<0.001) and maximum percent change in sCR (r=0.79; p<0.001) in these patients compared to their on-pump counterparts. ROC curve analysis was used to evaluate the prediction of worsening of AKI, AKIN stage 3, RRT, and AKIN stage 2 or 3. uAnCR is a very strong predictor in this group compared to the previous analysis. It predicted the development of both AKIN stage 3 and RRT with very high accuracy (AUC=0.93 and 0.86, respectively). The inventors did not present the ROC curves for composite outcomes that included death because all patients who died met the outcomes of AKIN stage 3 or RRT, and thus the ROC curves would have been identical to those evaluating the individual outcomes. However, given this and the other data, it is likely that uAnCR would be highly predictive of these composite outcomes. An important limitation of this analysis is that uAnCR was not predictive of worsening of AKI (FIG. 6). The AUC for this ROC curve was relatively high (0.77); although the p-value did not meet statistical significance, this is likely the result of the limited statistical power in the smaller dataset.

EARLY Study.

Urine proteins from four patients who did not develop AKI after cardiac surgery were compared to four patients that did. Preoperative sCr, bypass time, change in sCr and time after surgery to collection were not different between groups. The mean time after surgery to collection of the urine was 9 h in both groups. The mean change to maximum sCr in the no AKI group was 19±3% and in the AKI group was 171±38%. Proteomic analysis was done as described in the RRT study. The inventors identified 227 proteins with high confidence (FDR<0.1%). 11 proteins were statistically different between the groups.

RAT Study.

The goal of this study was to identify urine proteins that change in another model of AKI so that the information could be used to determine how generalizable the data from the human studies are. AKI was induced in rats by injection of glycerol. Serum creatinine peaked at 24 h after injection and then improved. Urine collected for 4 h before the 24 h time point was used for proteomic analysis of three control rats and three AKI rats as described for the human studies. 259 proteins were identified with high confidence (FDR<0.1%). 110 proteins were statistically different between groups.

Selection of Candidate Markers.

The inventors chose AKI markers based on their changes in the three proteomic studies (FIG. 8). The goal was to identify urine proteins that are likely to predict AKI across multiple injury models, species and time points. Selection of markers was based on the magnitude of difference between groups and p value of the difference. Some selected proteins were not seen in all three studies. Major emphasis in the selection was placed on the changes that were seen in the RRT study. Several candidate markers were chosen based on the differences seen in the EARLY study even though the changes in the RRT study were not as impressive. Findings in the RAT study were used to determine the generalizability of the changes in the candidate markers. Biological plausibility as an AKI biomarker was used as an additional criterion. The inventors chose 21 protein candidate markers as globally indicative of AKI across species and models. Fifteen of the biomarkers increased and six decreased.

Development of MRM Assays.

As an example of the development of a mass spectrometry quantitative assay, the inventors show the development of an assay for human haptoglobin. Absolute quantification by LC-MS/MS is referred to as selective reaction monitoring (SRM) if a product ion is monitored for quantification. Monitoring of multiple product ions from a fragmented peptide is known as multiple reaction monitoring (MRM). Monitoring a product ion provides added specificity especially in the case when two parent ions of nearly identical mass elute by liquid chromatography at similar times. A workflow for SRM begins with sample preparation where proteins are isolated and digested with a protease such as trypsin. To the mixture, one or more synthetic peptides resembling the target peptide of interest is added as an internal standard at a known concentration. The synthetic peptide is identical to the target peptide with the exception that one amino acid is comprised of stable isotopes of carbon (¹³C) and nitrogen (¹⁵N). Both peptides are chemically identical with respect to chromatographic separation and decomposition, but the stable isotope labeled peptide is heavier and is detected as a different m/z by the mass spectrometer. This is exemplified in FIG. 2B where a +2 charged tryptic peptide from the beta-chain of haptoglobin is shown. VTSIQDWVQK has a mass to charge ratio (m/z) of 602.3. Stable isotopes of carbon and nitrogen are incorporated into the c-terminal lysine residue of the internal standard peptide located at 606.3 m/z and result in a 4 m/z difference at a +2 charge between the endogenous and labeled peptides. Both peptides are fragmented sequentially in a collision cell and product ions are detected (FIG. 2C). Product ions are often detected as a +1 charge and the difference between unlabeled product ions and corresponding labeled product ions carrying the lysine residue are 8 m/z. At this point, the product ion chromatogram can be extracted for one or many of the product ions and area under the curves are compared between the unlabeled and labeled peptide (FIG. 2D). The ratio of the internal standard to the unknown target peptide provides an estimate of the absolute concentration directly or against an external standard curve, the later can be beneficial when the slope of the relationship deviates from unity (FIG. 2E). Although this example is a single peptide, more than one “proteotypic” peptide is more commonly measured to estimate the abundance of a given protein (Kuzyk et al., 2009; Selevsek et al., 2011). Data provided in this example were collected using an AB-SCIEX triple-TOF mass spectrometer, which is similar to measurements on a triple quadrupole mass spectrometer, but the former differs in that no single product ion is isolated in a stepwise manner. The inventors have used this approach to simultaneously measure six proteins in multiple urine samples and the technique can easily be extended to include measurement of over 100 proteins without loss of specificity.

### Example 2

The inventors will use a similar approach to generate the multiplexed MRM assay to use for rat AKI markers. As an example, the inventors show the development of a panel of AKI biomarker assays that have been tested by the Predictive Safety Testing Consortium (PSTC). The multiplexed assay consists of a panel of MRM assays to measure 6 nephrotoxicity markers in rats and determine the assay characteristics for each analyte to result in a 6-plex assay. The panel will include the following proteins: 6 urine proteins from the PSTC (Kim-1, Trefoil factor 3, albumin, β2-microglobulin, cystatin C and clusterin). The seventh PSTC marker is total urine protein concentration which is not an individual protein and will not be included in this assay. These proteins have been approved by the FDA and EMA for preclinical evaluation of nephrotoxicity.

TABLE 7
Peptides for measurement of 6 PSTC nephrotoxicity biomarker proteins

| | Protein | | | Peptide | | | Aliphatic | |
|---|---|---|---|---|---|---|---|---|
| Protein | ID | Peptide | | seen | MW | pl | index | GRAVY |
| Kim-1 | O54947 | 1 | VEIPGWFNDQK | ✓ | 1332.4 | 4.37 | 61.82 | −0.845 |
| | | | (SEQ ID NO: 2) | | | | | |
| | | 2 | GVVGHPVTIPCTYSTR | ✓ | 1686.9 | 8.23 | 78.75 | 0.231 |
| | | | (SEQ ID NO: 3) | | | | | |
| Trefoil factor | Q03191 | 1 | PLQETECTF | | 1067.1 | 3.80 | 43.33 | −0.489 |
| 3 | | | (SEQ ID NO: 4) | | | | | |
| | | 2 | VDCGYPTVTSEQCNNR | ✓ | 1785.9 | 4.37 | 36.25 | −0.881 |
| | | | (SEQ ID NO: 5) | | | | | |
| | | 3 | GCCFDSSIPNVPWCFK | ✓ | 1803.1 | 5.82 | 42.50 | 0.300 |
| | | | (SEQ ID NO: 6) | | | | | |
| Albumin | P02770 | 1 | LVQEVTDFAK | ✓ | 1149.3 | 4.37 | 107.00 | 0.170 |
| | | | (SEQ ID NO: 7) | | | | | |
| | | 2 | LGEYGFQNAVLVR | ✓ | 1465.6 | 6.00 | 112.31 | 0.269 |
| | | | (SEQ ID NO: 8) | | | | | |
| | | 3 | FPNAEFAEITK | ✓ | 1266.4 | 4.53 | 53.64 | −0.273 |
| | | | (SEQ ID NO: 9) | | | | | |
| | | 4 | GLVLIAFSQYLQK | ✓ | 1479.7 | 8.59 | 150.00 | 0.869 |
| | | | (SEQ ID NO: 10) | | | | | |
| | | 5 | DVFLGTFLYEYSR | ✓ | 1609.8 | 4.37 | 82.31 | 0.108 |
| | | | (SEQ ID NO: 11) | | | | | |
| β2-microglobulin | P07151 | 1 | TPQIQVYSR | ✓ | 1091.2 | 8.41 | 75.56 | −0.800 |
| | | | (SEQ ID NO: 12) | | | | | |
| Cystatin C | P14841 | 1 | ALDFAVSEYNK | ✓ | 1256.3 | 4.37 | 80.00 | −0.191 |
| | | | (SEQ ID NO: 13) | | | | | |
| | | 2 | LLGAPQEADASEEGVQR | ✓ | 1769.8 | 4.00 | 80.59 | −0.676 |
| | | | (SEQ ID NO: 14) | | | | | |
| | | 3 | GSNDAYHSR | | 1006 | 6.74 | 11.11 | −1.800 |
| | | | (SEQ ID NO: 15) | | | | | |
| Clusterin | P05371 | 1 | SLLNSLEEAK | ✓ | 1103.2 | 4.53 | 127.00 | −0.280 |
| | | | (SEQ ID NO: 16) | | | | | |
| | | 2 | ASGIIDTLFQDR | ✓ | 1335.4 | 4.21 | 105.83 | 0.042 |
| | | | (SEQ ID NO: 17) | | | | | |
| | | 3 | LFDSDPITVVLPEEVSK | ✓ | 1888.1 | 3.92 | 120.00 | 0.241 |
| | | | (SEQ ID NO: 18) | | | | | |

The approach involved generating a multiplexed assay that can be used to measure the 6 nephrotoxicity markers. Synthesized peptide standards measured together can be used to measure each marker concentration. Each standard peptide is further evaluated for chromatographic retention time, optimal collision energy, and product ion abundance. The inventors designed peptide sequences that will be used to quantify the six PSTC nephrotoxicity biomarker proteins. The inventors selected peptide sequences from the six proteins based on the following criteria: between 8 and 20 amino acids in length, unique to the protein of interest, tryptic peptides, avoidance of modified peptides except where synthetic versions of the modification were available and chemical indices suggesting strong ionization potential and solubility. The inventors attempted to choose 2-3 peptides for each protein. Preference was given to peptides that the inventors have seen previously in proteomic analysis of rat urine. Only one peptide from β-2 microglobulin was useful for MRM because all other potential tryptic peptides were too short, too long or included modified amino acids. The inventors selected five peptides for albumin since albumin is known to have multiple proteolytic peptides in the urine. In the final analysis, urinary albumin will be measured using an average of the values for all five urinary albumin peptides. The selected peptides for these six proteins are shown in Table 7. The inventors will have an unlabeled peptide synthesized for each of the sequences shown in the table. The peptides will be combined into a single composite mix containing an appropriate molar concentration of each peptide and analyzed for the ability of the peptides to ionize and thus be detected by a triple quadrupole MS or other mass spectrometer. The composite mix of peptides will be separated by liquid chromatography using a standardized gradient and the elution time of each peptide that will be determined. For each peptide the ideal declustering potential and collision energy will be determined. The peptide composite mix will be serially diluted into seven decreasing concentrations that bracket the expected urine concentration of the protein. The concentration for every peptide in each of the dilutions will be analyzed by LC-MS/MS using the optimized parameters. Three product ions for each parent ion (peptide) will be chosen for quantification. Area for each product ion will be extracted and analyzed using MultiQuant (AB-SCIEX). The linearity of each of the product ions as well as the linearity of the mean of the areas of the three product ions will be determined and ions with an R² of less than 0.99 will be rejected. If some of the selected peptides fail this test, the inventors will select replacement peptides using the same criteria. If necessary, alternative digestion enzymes will be used. For peptides that meet the criteria, the inventors will order the synthesis of isotopically labeled standard peptides that are chemically identical but 8 or 10 Da heavier (lysine or arginine, respectively) than the unlabeled peptides. A highly accurate determination of the concentration of the peptides will be done using amino acid analysis. New standard concentration curves will be generated using the labeled peptides. The inventors will perform the same analyses to generate standard curves for all peptides representing these six PSTC proteins. The standard curves generated for at least two peptides for each protein using up to three product ions from each peptide at a specific elution time will provide a highly specific and accurate assessment of the protein concentration for each protein in the multiplexed assay.

The inventors will also conduct in depth assay characterization and technical validation of the assay in urine. To determine the assay characteristics of each of the 6 proteins in the 6-plex nephrotoxicity panel assay the inventors will use commercially available rat urine (Bioreclamation, New York, N.Y.) or rat urine the inventors have previously banked from rats with two models of acute kidney injury (ischemia/reperfusion and glycerol injection) and from control rats. The urine is in three pools (control, I/R and glycerol injection). The inventors will use urine from these three pools to determine the measurement characteristics of the assays. The peptide composite mix containing isotopically labeled peptides will be added in an appropriate concentration to the urine pools for each of the characterization studies. Analytical method validation is the process of defining the performance characteristics of a biomarker assay. The inventors will characterize the dynamic range, limits of quantification (LOQ), accuracy and precision, matrix effects and short-term stability for each of the analytes. Validation Samples/Quality Control Samples. The inventors will use the three pooled urine samples in each of the studies. For the follow up studies in the phase 2 SBIR application the inventors will select larger volumes of urine which will comparable to the pooled quality control samples used in these analyses. Assay Dynamic Range. The dynamic range for the assays will be determined using double-labeled synthetic isotopic standards to urine and assay buffer. The double-labeled standards will be synthesized commercially using ¹³C and ¹⁵N. For instance if the c-terminal residue of a peptide is lysine and the adjacent peptide is glycine, both would be labeled. The use of two labeled peptides will enable us to determine the LOQ and detection within the urine matrix. The limit of detection will be defined as the lowest concentration of the double-labeled peptide that can be added where the value is greater than three SD above background. The lower LOQ and the upper LOQ will be defined as the concentrations for which the precision (determined by the coefficient of variation calculated from measurement of four replicates) is better than 20% (DeSilva et al., 2003). Dynamic range studies will be repeated using the two AKI and the control urine samples to confirm that the % CVs in urine are consistent in AKI and non AKI samples and with those seen in assay buffer. Accuracy. Accuracy is the assessment of how close the measurement is to the true value. It will be determined by measurement of the proteins after addition of recombinant protein to each of the urine validation samples. The inventors have determined that recombinant protein is available for all 6 of the proteins in the nephrotoxicity assay. Accuracy (% relative accuracy) will be expressed as the percent deviation from the nominal reference value (added minus endogenous concentration) and calculated using spiked standard from four replicates in each of the three pooled samples. Precision. Precision is a measure of the reproducibility of the measurement. Two types of precision will be determined: repeatability (agreement between repeated measurements of the same sample by the same operator) and intermediate precision (agreement between measures in different runs by different operators). Repeatability will be assessed by measuring the endogenous concentrations in each of the three samples using four analyses by the same operator on the same day. Intermediate precision will be assessed by measurement of the six proteins in the three samples by different operators separated by at least three weeks. Between the measurements in the intermediate precision analysis the inventors will change the analytical columns on the LC as well as remix all of the buffers. Each analysis will be done in four replicates for each of the three validation samples. Method precision will be expressed as % CV. Parallelism documents the relationship between measurement of the proteins in the urine matrix and in the assay buffer in which the standard curve is made. The inventors will measure the concentration of recombinant protein in assay buffer and in urine matrix containing added recombinant protein using each of the three pools. Stability. The inventors will determine short-term stability of the analytes as measured by the assay using the three pooled specimens. The inventors will compare concentrations of the analytes measured in freshly thawed aliquots of urine with the concentration in aliquots of urine left at room temperature for 1 and days and stored at 4° C. for 1 day and 7 days. In the studies in phase 2 of this project the inventors will do a more comprehensive analysis of the stability of the analytes including the need for protease inhibitors, the effect of centrifugation before freezing the samples, comparison of never frozen samples with other conditions and the effects of various durations of freezing at −20 and −80° C.

The inventors will conduct initial assay performance optimization for individual peptides representative of 30 additional AKI markers in rats to expand upon the 6-plex assay. The goal of this study is to expand the number of urine markers for estimating kidney injury in addition to the 6-plex nephrotoxicity assay. The inventors will evaluate a total of 60 standard peptides representing 30 urine proteins for solubility, retention time, optimal declustering potential and collision energy, and product ion abundance. Twenty-one of these urine proteins represent novel markers the inventors have discovered and upon preliminary optimization the inventors intend to incorporate these markers into the 6-plex panel 1 assay, thereby creating the larger AKI assay to be technically validated. Proteins evaluated in these experiments include:

6 Urine proteins initially evaluated by PSTC but not pursued (calbindin d28, NGAL, podocin, renal papillary antigen 1, TIMP-1, VEGF). There is a large body of evidence that these proteins may be AKI biomarkers but they were not evaluated by the PSTC. The inventors will include them in the multiplexed assay.

23 Novel AKI proteins the inventors identified by combining data from 3 proteomic analyses (Angiotensinogen, Apolipoprotein A-IV, Pigment epithelium-derived factor, Thymosin beta-4, Insulin-like growth factor-binding protein 1, Myoglobin, Vitamin D binding protein, Complement C4-B, Profilin-1, alpha-1 antitrypsin, fibrinogen alpha chain, Glutathione peroxidase 3, Superoxide dismutase [Cu—Zn], Complement C3, Antithrombin III, Neutrophil defensin 1, Lysozyme C, Non-secretory ribonuclease, Secreted Ly-6/uPAR-related protein 1, Uromodulin, Polymeric IgG receptor, CD59 glycoprotein, Hepcidin).

3 Other candidate AKI urine proteins (Cyr61, NHE-3, L-FABP) A number of studies suggest that these proteins may be markers of AKI but they were not considered by the PSTC and have not been extensively evaluated.

### Example 3

### Urinary Angiotensinogen Predicts Outcomes in AKI Patients in the ICU

Methods:

Urinary angiotensinogen was measured by ELISA in urine samples from ICU patients with AKI of diverse causes (n=40; Table 1). ROC curves were used to evaluate the ability of urine creatinine corrected angiotensinogen to predict the following outcomes: worsening of AKI, AKIN stage 3 AKI, need for renal replacement therapy (RRT), AKIN stage 3 AKI or death, and RRT or death.

Results:

Patients who met the primary outcome of RRT/death had a nearly twelve-fold increase in median uAnCR compared to those who did not (133.3 ng/mg versus 11.4 ng/mg). ROC curve analysis demonstrated that uAnCR was a strong predictor of this outcome (AUC=0.79). In addition to the primary outcome, the inventors found that uAnCR was a modest predictor of the composite outcome AKIN stage 3 AKI or death (AUC=0.71). Finally, the inventors found that patients with high concentrations of uAnCR had increased length of stay in the hospital compared to those with low uAnCR (22 days versus 7 days; p=0.03), and uAnCR was a strong predictor of hospital discharge≦7 days from sample collection (AUC=0.79).

### CONCLUSIONS

These data confirm the potential of angiotensinogen as a prognostic AKI biomarker, and this data demonstrates that it is predictive of outcomes in the setting of AKI secondary to causes other than cardiac surgery.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,376,110
U.S. Pat. No. 4,816,567
U.S. Pat. No. 5,034,506
U.S. Pat. No. 5,235,033
U.S. Pat. No. 5,364,790
U.S. Pat. No. 5,475,610
U.S. Pat. No. 5,527,510
U.S. Pat. No. 5,547,861
U.S. Pat. No. 5,593,867
U.S. Pat. No. 5,675,063
U.S. Pat. No. 5,681,741
U.S. Pat. No. 5,716,784
U.S. Pat. No. 5,853,990
U.S. Pat. No. 6,136,610
U.S. Pat. No. 6,268,490
U.S. Pat. No. 6,346,384
U.S. Pat. No. 6,770,748
U.S. Pat. No. 7,122,799
U.S. Pat. No. 7,344,893
U.S. Pat. No. 7,371,582
U.S. Pat. No. 7,429,487
U.S. Patent Appln. 2005/0047972
U.S. Patent Appln. 2005/0250141
U.S. Patent Publn. 2002/0115080
U.S. Patent Publn. 2005/0107325
U.S. Patent Publn. 2005/0182005
U.S. Patent Publn. 2008/0108091
Allred et al., Am. J. Physiol. Renal. Physiol., 279(4):F636-645, 2000.
Arora et al., Clin. J. Am. Soc. Nephrol., 3(5):1266-1273, 2008.
Askenazi et al., J. Pediatr., 2012 (Epub ahead of print).
Bell et al., Nucleic Acids Res., 14:21:8427-8446, 1986.
Bellomo et al., Crit. Care, 8(4):R204-212, 2004.
Benedetto et al., Ann. Thorac. Surg., 86(4):1160-1165, 2008.
Brenner et al., N. Engl. J. Med., 345(12):861-869, 2001.
Chertow et al., Am. J. Med., 104(4):343-348, 1998.
Chertow et al., J. Am. Soc. Nephrol., 16(11):3365-3370, 2005.
Coca et al., Am. J. Kidney Dis., 53(6):961-973, 2009.
Cruz et al., Crit. Care, 13(3):211, 2009.
Dakna et al., BMC Bioinformatics, 11:594, 2010.
David et al., Biochemistry 12:1014, 1974
DeSilva et al., Pharm. Res., 20(11):1885-1900, 2003.
Di Paolo et al., Nephrol. Dial. Transplant., 12:12:2687-2693, 1997.
du Cheyron et al., Crit. Care, 36(12):3178-3183, 2008.
Englberger et al., Crit. Care, 15(1):R16, 2011.
Finney et al., Clin Chem. 43(6 Pt 1):1016-22, 1997.
Goding, Monoclonal Antibodis: Principles and Practice, 2^{nd} Ed., Academic Press, Orlando, Fla., 60-61, 71-74, 1986.
Haase et al., Crit. Care, 12(4):R96, 2008.
Hall et al., Clin. J. Am. Soc. Nephrol., 6(12):2740-2749, 2011.
Han et al., Kidney Int., 62(1):237-244, 2002.
Herget-Rosenthal et al., Kidney Int., 66(3):1115-1122, 2004.
Higuchi et al., Biotechnology, 10:413-417, 1992.
Higuchi et al., Biotechnology, 11:1026-1030, 1993.
Humes et al., J. Clin. Invest., 84:6:1757-1761, 1989.
Hunter et al., Nature 144:945, 1962
Inglis et al., Appl. Environ. Microbiol., 70(4):2296-2306, 2004.
Keller et al., Anal. Chem., 74(20):5383-5392, 2002.
Kim et al., Proc. Natl. Acad. Sci. USA, 92(7):2735-2739, 1995.
Kohler & Milstein, Nature, 256:495-497, 1975.
Koyner et al., Clin. J. Am. Soc. Nephrol., 5(12):2154-2165, 2010.
Koyner et al., J. Am. Soc. Nephrol., 23(5):905-914, 2012.
Koyner et al., Kidney Int., 74(8):1059-1069, 2008.
Krutzfeldt et al. J. Biol. Chem., 280:16635-16641, 2005.
Kuzyk et al., Mol. Cell. Proteomics, 8(8):1860-1877, 2009.
Lafrance and Miller, J. Am. Soc. Nephrol., 21(2):345-352, 2010.
Leach M et al., British Journal of Pharmacology., 125:817-825, 1998.
Lewis et al., N. Engl. J. Med., 329(20):1456-1462, 1993.
Liangos et al., Biomarkers, 14(6):423-431, 2009.
Lo et al., Kidney Int., 76(8):893-899, 2009.
Loef et al., J. Am. Soc. Nephrol., 16(1):195-200, 2005.
Logan et al., In: Real-Time PCR: Current Technology and Applications, Caister Academic Press, ISBN 978-1-904455-39-4, 2009.
Mackay, In: Real-Time PCR in Microbiology, Caister Academic Press, ISBN 978-1-904455-18-9, 2007.
Manual of Histological Staining Method of the Armed Forces Institute of Pathology,” 3^{rd} Ed. (1960)
Mehta et al., Crit. Care, 11(2):R31, 2007.
Mehta et al., Kidney Int., 66(4):1613-1621, 2004.
Melnikov et al., J. Clin. Invest., 107(9):1145-1152, 2001.
Mishra et al., J. Am. Soc. Nephrol., 14(10):2534-2543, 2003.
Mishra et al., Lancet., 365(9466):1231-1238, 2005.
Morrison et al., Proc. Natl. Acad., Sci. USA, 81:6851-6833, 1984.
Munson and Pollard, Anal. Biochem., 107:220, 1980.
Nash et al., Am. J. Kidney Dis., 39(5):930-936, 2002.
Nesvizhskii et al., Anal. Chem., 75(17):4646-4658, 2003.
Nicol et al., Am J Kidney Dis. 58(4):685-7, 2011.
Nolan et al., Nat. Protoc., 1:1559-1582, 2006.
Nygren, J. Histochem. Cytochem. 30:407-412, 1982
Pain et al., J. Immunol. Meth. 40:219, 1981;
Parikh et al., Am. J. Kidney Dis., 43(3):405-414, 2004.
Parikh et al., J. Am. Soc. Nephrol., 22(9):1737-1747, 2011a.
Parikh et al., J. Am. Soc. Nephrol., 22(9):1748-1757, 2011b.
Parikh et al., Kidney Int., 70(1):199-203, 2006.
Park et al., Oncol. Rep., 20(3): 597-604, 2008.
Pepe, In: The statistical evaluation of medical tests for classification and prediction, Oxford University Press, 2004.
Plataki et al., Clin. J. Am. Soc. Nephrol., 6(7):1744-1751, 2011.
Portilla et al., Kidney Int., 73(4):465-472, 2008.
Ricci et al., Nat. Rev. Nephrol., 7(4):201-208, 2011.
Rosner et al., J. Intensive Care Med., 23(1):3-18, 2008.
Sails, In: Real-Time PCR: Current Technology and Applications, Caister Academic Press, ISBN 978-1-904455-39-4, 2009.
Selevsek et al., Proteomics, 11(6):1135-1147, 2011.
Soutschek et al., Nature, 432(7014):173-178, 2004.
Spieker-Polet et al., Proc. Natl. Acad. Sci. USA, 92:9348-9352, 1995.
Uchino et al., JAMA, 294(7):813-818, 2005.
Umstead et al., Pediatr. Res., 67(6):641-649, 2010.
Velez, Nat. Clin. Pract. Nephrol., 5(2):89-100, 2009.
Venkatachalam et al., Am. J. Physiol. Renal Physiol., Mar. 3, 2010 (Epub ahead of print).
Wagener et al., Am. J. Kidney Dis., 52(3):425-433, 2008.
Waikar et al., J. Am. Soc. Nephrol., 17(4):1143-1150, 2006.
Wawrik et al., Appl. Environ. Microbiol., 68:3771-3779, 2002.
Yoo et al., Circ. J., 74(9):1852-1858, 2010.
Zola, In: Molecular Antibodies: A Manual of Techniques, CRC Press, Inc., pp 147-158, 1987

1. A method for determining an increased risk of developing a nephropathy or kidney disease in a subject, comprising measuring at least one protein in a urine sample from said subject, wherein said protein is selected from the group consisting of:
(a) angiotensinogen, apolipoprotein A-IV, pigment epithelium-derived factor, thymosin β4, insulin-like growth factor-binding protein 1, myoglobin, vitamin D binding protein, complement C4-B, profilin-I, alpha-1 antitrypsin, fibrinogen alpha chain, glutathione peroxidase 3, superoxide dismutase [Cu—Zn], complement C3, antithrombin neutrophil defensin 1, and
(b) non-secretory ribonuclease, secreted Ly-6/uPAR-related protein 1, pro-epidermal growth factor precursor (pro-EGF protein), and CD59 glycoprotein;
wherein an increase in level of a protein from group (a) or a decrease in level of a protein from group (b) in said urine sample relative to a reference level indicates that the subject has an increased risk of developing the nephropathy or kidney disease.

2. The method of claim 1, wherein said protein is selected from the group consisting of:
(a) apolipoprotein A-IV, thymosin β4, insulin-like growth factor-binding protein 1, vitamin D binding protein, profilin-1, glutathione peroxidase 3, superoxide dismutase [Cu—Zn], neutrophil defensin 1, and
(b)) non-secretory ribonuclease, secreted Ly-6/uPAR-related protein 1, pro-epidermal growth factor precursor (pro-EGF protein), and CD59 glycoprotein.

3. The method of claim 1, further comprising administering a kidney therapy or kidney therapeutic to the subject if the subject has an increased risk of developing the nephropathy or kidney disease.

4. The method of claim 1, further comprising preparing a report of said measuring.

5. The method of claim 1, wherein the nephropathy or kidney disease is acute kidney injury (AKI), a progressive or worsening acute kidney injury, an early AKI, a mild AKI, a moderate AKI, a severe AKI, diabetic nephropathy, acute tubular necrosis, acute interstitial nephritis, a glomerulonephropathy, a glomerulonephritis, a renal vasculitis, an obstruction of the renal artery, a renal ischemic injury, a tumor lysis syndrome, rhandomyolysis, a urinary tract obstruction, a prerenal azotemia, a renal vein thrombosis, a cardiorenal syndrome, a hepatorenal syndrome, a pulmonary-renal syndrome, an abdominal compartment syndrome, an injury from a nephrotoxic agent, or a contrast nephropathy.

6. The method of claim 1, wherein the protein is angiotensinogen.

7. The method of claim 6, wherein said measuring comprises measuring the urine angiotensinogen to creatinine ratio (uAnCR), wherein an increase in the uAnCR relative to a reference level indicates that the subject has an increased risk of severe AKI.

8. The method of claim 1, further comprising measuring creatinine concentration in the urine sample.

9. The method of claim 1, wherein a cardiac surgery is or has been performed on the subject.

10. The method of claim 1, wherein said measuring comprises measuring a second protein from group (a) or group (b).

11. The method of claim 10, wherein said measuring comprises measuring a third protein from group (a) or group (b).

12. The method of claim 11, wherein said measuring comprises measuring all proteins from group (a) and group (h).

13. The method of claim 1, further comprising measuring a second protein in said urine sample, wherein said protein is selected from the group consisting of:
(c) lysozyme c and albumin; and
(d) uromodulin, hepcidin, and polymeric immunoglobulin receptor;
wherein an increase in level of a protein from group (c) or a decrease in level of a protein from group (d) in said urine sample relative to a reference level indicates that the subject has an increased risk of developing acute kidney injury.

14. The method of claim 1, further comprising measuring urea nitrogen or creatinine in the blood of the subject.

15. The method of claim 1, wherein the subject is a human patient.

16. The method of claim 1, wherein the kidney disease comprises worsening of AKI, AKIN stage 2 AKI, AKIN stage 3 AKI, a need for renal replacement therapy, or death.

17. The method of claim 1, wherein the subject has diabetes, prediabetes, sepsis, an infection, a systemic inflammatory response syndrome, hypovolemia, hypotension, a cardiac disease, a liver disease, a pulmonary disease, a cancer, a traumatic injury, a cardiac surgery, a noncardiac surgery, an abdominal cavity surgery, an aneurysm repair surgery or is given a potentially nephrotoxic agent.

18. The method of claim 1, wherein the subject has substantially no acute kidney injury when the urine sample is obtained from the subject.

19. The method of claim 1, further comprising monitoring the response to a treatment for acute kidney injury in the patient.

20. The method of claim 1, wherein said measuring comprises mass spectrometry, LC-MS/MS, MALDI-MS/MS, MALDI-MS, selected reaction monitoring (SRM), multiple reaction monitoring (MRM), Surface enhanced laser desorption/ionization (SELDI) or capillary electrophoresis mass spectrometry (CE-MS).

21. The method of claim 1, wherein said measuring comprises an immunoassay method, an immunohistochemistry assay, a radioimmunoassay (RIA), an immunoradiometric assay, a Western blot analysis, a fluoroimmunoassay, an automated quantitative analysis (AQUA) system assay, spectroscopy, spectrophotometry, a lateral flow assay, a chemiluminescent labeled sandwich assay, a nephelometry assay, an enzyme-linked immunosorbent assay (ELISA), a chemiluminescent assay, a bioluminescent assay, a gel electrophoresis, or a nephelometry assay.

22. The method of claim 21, wherein said measuring comprises an ELISA assay.

23. A method for determining an increased risk of developing a progressing or worsening diabetic nephropathy or kidney disease in a subject, comprising measuring angiotensinogen in a urine sample from said subject, wherein an increased angiotensinogen level in said urine sample relative to a reference level or control sample indicates that the subject has an increased risk of developing a kidney disease or developing the progressing or worsening nephropathy or kidney disease, and wherein the subject has diabetes.

24. The method of claim 23, wherein the subject has at least a mild diabetic nephropathy or kidney disease when the urine sample is obtained from the subject.

25. The method of claim 23, wherein said diabetes has type 1 diabetes.

26. The method of claim 23, wherein said diabetes has type 2 diabetes.

27. The method of claim 26, wherein said measuring comprises an ELISA assay.

28. The method of claim 23, wherein said measuring is selected from the group consisting of mass spectrometry, multiple reaction monitoring (MRM), selected reaction monitoring, single reaction monitoring, an immunoassay method, an immunohistochemistry assay, a radioimmunoassay (RIA), an immunoradiometric assay, a Western blot analysis, a fluoroimmunoassay, an automated quantitative analysis (AQUA) system assay, spectroscopy, spectrophotometry, a lateral flow assay, a chemiluminescent labeled sandwich assay, and an enzyme-linked immunosorbent assay (ELISA), a chemiluminescent assay, a bioluminescent assay, a gel electrophoresis, or a nephelometry assay.

29. A kit for determining the likelihood of acute kidney injury (AKI) in a mammalian subject, comprising an antibody that specifically binds a protein selected from the group consisting of:
(a) angiotensinogen, apolipoprotein A-IV, pigment epithelium-derived factor, thymosin β4, insulin-like growth factor-binding protein 1, myoglobin, vitamin D binding protein, complement C4-B, profilin-1, alpha-i antitrypsin, fibrinogen alpha chain, glutathione peroxidase 3, superoxide dismutase [Cu—Zn], complement C3, antithrombin neutrophil defensin 1, and
(b) non-secretory ribonuclease, secreted Ly-6/uPAR-related protein 1, pro-epidermal growth factor precursor (pro-EGF protein), and CD59 glycoprotein;
and a suitable container means.

30-40. (canceled)
